(19) United States
(12) Patent Application Publication
(10) Pub. No.: US 2003/0114437 A1
(43) Pub. Date: Jun. 19, 2003

(54) Protease inhibitors

(76) Inventors: Maxwell D. Cummings, Strafford, PA (US); Robert W. Marquis Jr, Wayne, PA (US); Yu Ru, Wayne, PA (US); Scott K. Thompson, Phoenixville, PA (US); Daniel F. Veber, Ambler, PA (US); Dennis S. Yamashita, Wayne, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/258,053

(22) PCT Filed: Apr. 17, 2001
(86) PCT No.: PCT/US01/12326

(51) Int. Cl. A61K 31/55; C07D 413/02; C 07D 4 3/02; C07D 223/12

(52) U.S. Cl. 514/212.03; 514/212.08; 540/524; 540/527

(57) ABSTRACT

The present invention provides methods which use 4-amino-azepan-3-one protease inhibitors of cathepsin S in the treatment of diseases in which cathepsin S is implicated, especially treatment or prevention of autoimmune disease; treatment or prevention of a disease state caused by the formation of atherosclerotic lesions and complications arising therefrom; and diseases requiring inhibition, for therapy, of a class II MHC-restricted immune response, inhibition of an asthmatic response, inhibition of an allergic response, inhibition of immune response against a transplanted organ or tissue, or inhibition of elastase activity in atheroma, and novel compounds for use therewith.

### FIELD OF THE INVENTION

This invention relates in general to the use of 4-amino-azepan-3-one protease inhibitors, particularly such inhibitors of cathepsin S, in the treatment of diseases in which cathepsin S is implicated, especially treatment or prevention of autoimmune disease; treatment or prevention of a disease state caused by the formation of atherosclerotic lesions and complications arising therefrom; and diseases requiring inhibition, for therapy, of a class II MHC-restricted immune response, inhibition of an asthmatic response, inhibition of an allergic response, inhibition of immune response against a transplanted organ or tissue, or inhibition of elastase activity in atheroma; and novel compounds for use therewith.

### BACKGROUND OF THE INVENTION

Cathepsins are a family of enzymes which are part of the papain superfamily of cysteine proteases. Cathepsins K, B, H, L, N and S have been described in the literature.

Cathepsins function in the normal physiological process of protein degradation in animals, including humans, e.g., in the degradation of connective tissue. However, elevated levels of these enzymes in the body can result in pathological conditions leading to disease. Thus, cathepsins have been implicated as causative agents in various disease states, including but not limited to, infections by pneumocystis carinii, trypsanoma cruzi, trypsanoma brucei brucei, and Crithidia fusiculata; as well as in schistosomiasis, malaria, tumor metastasis, metachromatic leukodystrophy, muscular dystrophy, amytrophy, and the like. See International Publication Number WO 94/04172, published on Mar. 3, 1994, and references cited therein. See also European Patent Application EP 0 603 873 A1, and references cited therein. Two bacterial cysteine proteases from P. gingivallis, called gingipains, have been implicated in the pathogenesis of gingivitis. Potempa, J., et al. (1994) Perspectives in Drug Discovery and Design, 2, 445-458. Cathepsin K is believed to play a causative role in diseases of excessive bone or cartilage loss. See International Publication Number WO 97/16433, published on May 9, 1997, and references cited therein.

Pathological levels of cathepsin S have been implicated in a variety of disease states. For instance, mice treated with inhibitor exhibited attenuated antibody response indicating that selective inhibition of cathepsin S may provide a therapeutic strategy for asthma and autoimmune disease processes. Riese, Richard J., et al., J. Clin. Invest. 1998 101(11), 2351-2363. Thus, selective inhibition of cathepsin S may provide an effective treatment for diseases requiring, for therapy or prevention: inhibition of a class II MHC-restricted immune response; treatment and/or prevention of an autoimmune disease state such as rheumatoid arthritis, multiple sclerosis, juvenile-onset diabetes, sytemic lupus erythematosus, discoid lupus erythematosus, pemphigus vulgaris, pemphigoid, Grave's disease, myasthenia gravis, Hashimoto's thyroiditis, scleroderma, dermatomysositis, Addison's disease, pernicious anemia, primary myxoedema, thyrotoxicosis, autoimmune atrophic gastritis, stiff-man syndrome, Goodpasture's syndrome, sympathetic opthalamia, phacogenic uveitis, autoimmune haemolytic anaemia, idiopathic thrombocytopenic purpura, idiopathic leucopenia, primary biliary cirrhosis, active chronic hepatitis, cryptogenic cirrhosis, ulcerative colitis, Sjogren's syndrome, and mixed connective tissue diease; inhibition of an asthmatic response; inhibition of an allergic response; inhibition of immune response against transplanted organ or tissue (see I. Roitt, J. Brostoff, D. Male, Immunology, Fifth Edition, 1998, p.368; R. J. Riese, et al Immunity, 1996, 4, 357-366; G P Shi, et al Immunity 1999, 10, 197-206; T. Nakagawa, et al Immunity 1999, 10, 207-217; and International Publication No. WO 97/40066); inhibition of elastase activity in atheroma; and treatment or prevention of a disease state caused by the formation of atherosclerotic lesions or complications arising therefrom (G. K. Sukhova, et al J. Clin. Invest. 1998, 102, 576).

Several classes of cysteine protease inhibitors are known. Palmer et. al. (1995) J. Med. Chem., 38, 3193, disclose certain vinyl sulfones which irreversibly inhibit cysteine proteases, such as the cathepsins B, L, S, O2 and cruzain. Other classes of compounds, such as aldehydes, nitriles, α-ketocarbonyl compounds, halomethyl ketones, diazomethyl ketones, (acyloxy)methyl ketones, ketomethylsulfonium salts and epoxy succinyl compounds have also been reported to inhibit cysteine proteases. See Palmer, id, and references cited therein.

U.S. Pat. No. 4,518,528 discloses peptidyl fluoromethyl ketones as irreversible inhibitors of cysteine protease. Published International Patent Application No. WO 94/04172, and European Patent Application Nos. EP 0 525 420 A1, EP 0 603 873 A1, and EP 0 611 756 A2 describe alkoxymethyl and mercaptomethyl ketones which inhibit the cysteine proteases cathepsins B, H and L. International Patent Application No. PCT/US94/08868 and and European Patent Application No. EP 0 623 592 A1 describe alkoxymethyl and mercaptomethyl ketones which inhibit the cysteine protease IL-1βconvertase. Alkoxymethyl and mercaptomethyl ketones have also been described as inhibitors of the serine protease kininogenase (International Patent Application No. PCT/GB91/01479).

Azapeptides which are designed to deliver the azaamino acid to the active site of serine proteases, and which possess a good leaving group, are disclosed by Elmore et al., Biochem. J., 1968, 107, 103, Garker et al., Biochem. J., 1974, 139, 555, Gray et al., Tetrahedron, 1977, 33, 837, Gupton et al., J. Biol. Chem., 1984, 259, 4279, Powers et al., J. Biol. Chem., 1984, 259, 4288, and are known to inhibit serine proteases. In addition, J. Med. Chem., 1992, 35, 4279, discloses certain azapeptide esters as cysteine protease inhibitors.

Antipain and leupeptin are described as reversible inhibitors of cysteine protease in McConnell et al., J. Med. Chem., 33, 86; and also have been disclosed as inhibitors of serine protease in Umezawa et al., 45 Meth. Enzymol. 678. E64 and its synthetic analogs are also well-known cysteine protease inhibitors (Barrett, Biochem. J., 201, 189, and Grinde, Biochem. Biophys. Acta,, 701, 328).

1,3-diamido-propanones have been described as analgesic agents in U.S. Pat. Nos. 4,749,792 and 4,638,010.

A variety of cysteine and serine protease inhibitors, especially of cathepsin K, have been disclosed in International Publication Number WO 97/16433, published on May 9, 1997.

We have now discovered that certain 4-amino-azepan-3-one compounds inhibit cathepsin S, and are useful in the treatment of diseases in which cathepsin S is implicated.

### SUMMARY OF THE INVENTION

An object of the present invention is to provide methods of treatment which use 4-amino-azepan-3-one carbonyl protease inhibitors of cathepsin S of Formula I and which are useful for treating diseases which may be therapeutically modified by altering the activity of cathepsin S.

In a particular aspect, the methods of this invention are especially useful for treatment or prevention of autoimmune disease; treatment or prevention of a disease state caused by the formation of atherosclerotic lesions and complications arising therefrom; and diseases requiring inhibition, for therapy, of a class II MHC-restricted immune response, inhibition of an asthmatic response, inhibition of an allergic response, inhibition of immune response against a transplanted organ or tissue, or inhibition of elastase activity in atheroma.

Another object of the present invention is to provide novel compounds for use in the present methods.

### DETAILED DESCRIPTION OF THE INVENTION

The present invention provides a method of inhibiting cathepsin S comprising administering to an animal, particularly a mammal, most particularly a human being in need thereof, an effective amount of a compound of Formula I:

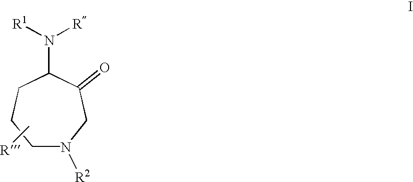

wherein:

R¹ is selected from the group consisting of:

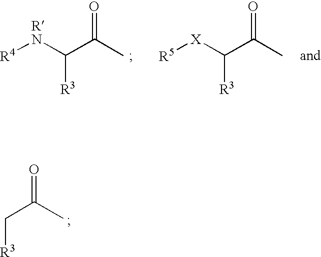

R² is selected from the group consisting of: H, C₁₋₆alkyl, C₃₋₆cycloalkyl-C₀₋₆alkyl, Ar—C₀₋₆alkyl, Het-C₀₋₆alkyl, R⁹C(O)—, R⁹C(S)—, R⁹SO₂—, R⁹OC(O)—, R⁹R¹¹NC(O)—, R⁹R¹¹NC(S)—, R⁹(R¹¹)NSO₂—

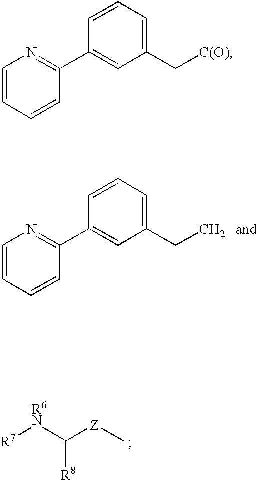

R³ is selected from the group consisting of: H, C₁₋₆aLkyl, C₃₋₆cycloalkyl-C₀₋₆alkyl, C₂₋₆alkenyl, C₂₋₆alkynyl, HetC₀₋₆alkyl, ArC₀₋₆alkyl, Ar—ArC₀₋₆alkyl, Ar-HetC₀₋₆alkyl, Het-ArC₀₋₆alkyl, and Het-HetC₀₋₆alkyl;

R³ and R′ may be connected to form a pyrrolidine, piperidine or morpholine ring;

R⁴ is selected from the group consisting of: H, C₁₋₆alkyl, C₃₋₆cycloalkyl-C₀₋₆alkyl, Ar—C₀₋₆alkyl, Het-C₀₋₆alkyl, R⁵C(O)—, R⁵C(S)—, R⁵SO₂—, R⁵OC(O)—, R⁵R¹³NC(O)—, and R⁵R¹³NC(S)—;

R⁵ is selected from the group consisting of: H, C₁₋₆alkyl, C₂₋₆alkenyl, C₂₋₆alkynyl, C₃₋₆cycloalkyl-C₀₋₆alkyl, Ar—C₀₋₆alkyl and Het-C₀₋₆alkyl;

R⁶ is selected from the group consisting of: H, C₁₋₆alkyl, C₃₋₆cycloalkyl-C₀₋₆alkyl, Ar—C₀₋₆alkyl, and Het-C₀₋₆alkyl;

R⁷ is selected from the group consisting of: H, C₁₋₆alkyl, C₃₋₆cycloalkyl-C₀₋₆alkyl, Ar—C₀₋₆alkyl, Het-C₀₋₆alkyl, R¹⁰C(O)—, R¹⁰C(S)—, R¹⁰SO₂—, R¹⁰OC(O)—, R¹⁰R¹⁴NC(O)—, and R¹⁰R¹⁴NC(S)—;

R⁸ is selected from the group consisting of: H, C₁₋₆alkyl, C₂₋₆alkenyl, C₂₋₆alkynyl, HetC₀₋₆alkyl and ArC₀₋₆alkyl;

R⁹ is selected from the group consisting of: C₁₋₆alkyl, C₃₋₆cycloalkyl-C₀₋₆alkyl, Ar—C₀₋₆alkyl and Het-C₀₋₆alkyl;

R¹⁰ is selected from the group consisting of: C₁₋₆alkyl, C₃₋₆cycloalkyl-C₀₋₆alkyl, Ar—C₀₋₆alkyl and Het-C₀₋₆alkyl;

R¹¹ is selected from the group consisting of: H, C₁₋₆alkyl, Ar—C₀₋₆alkyl, and Het-C₀₋₆alkyl;

R¹² is selected from the group consisting of: H, C₁₋₆alkyl, Ar—C₀₋₆alkyl, and Het-C₀₋₆alkyl;

R¹³ is selected from the group consisting of: H, C₁₋₆alkyl, Ar—C₀₋₆alkyl, and Het-C₀₋₆alkyl;

R¹⁴ is selected from the group consisting of: H, C₁₋₆alkyl, Ar—C₀₋₆alkyl, and Het-C₀₋₆alkyl;

R′ is selected from the group consisting of: H, C₁₋₆alkyl, Ar—C₀₋₆alkyl, and Het-C₀₋₆alkyl;

R″ is selected from the group consisting of: H, C₁₋₆alkyl, Ar—C₀₋₆alkyl, or Het-C₀₋₆alkyl;

R′″ is selected from the group consisting of: H, C₁₋₆alkyl, C₃₋₆cycloalkyl-C₀₋₆alkyl, Ar—C₀₋₆alkyl, and Het-C₀₋₆alkyl;

X is selected from the group consisting of: CH₂, S, and O;

Z is selected from the group consisting of: C(O) and CH₂;

and pharmaceutically acceptable salts, hydrates and solvates thereof.

In compounds of Formula I, R¹ is preferably

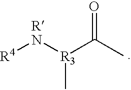

In such compounds:

R³ is selected from the group consisting of: H, C₁₋₆alkyl, C₃₋₆cycloalkyl-C₀₋₆alkyl,C₂₋₆alkenyl, C₂₋₆alkynyl, Het-C₀₋₆alkyl and Ar—C₀₋₆alkyl, preferably C₃₋₆cycloalkyl-C₀₋₆alkyl and C₁₋₆alkyl, especially selected from the group consisting of: cyclohexylmethyl and 2,2-dimethyl propyl, more preferably C₃₋₆cycloalkyl-C₀₋₆alkyl, most preferably cyclohexylmethyl;

R⁴ is selected from the group consisting of: H, C₁₋₆alkyl, C₃₋₆cycloalkyl-C₀₋₆alkyl, Ar—C₀₋₆alkyl, Het-C₀₋₆alkyl, R⁵C(O)—, R⁵C(S)—, R⁵SO₂—, R⁵OC(O)—, R⁵R¹³NC(O)—, and R⁵R¹³NC(S)—, preferably R⁵C(O)—.

R⁵ is selected from the group consisting of: C₁₋₆alkyl, C₂₋₆alkenyl, C₂₋₆alkynyl, C₃₋₆cycloalkyl-C₀₋₆alkyl, Ar—C₀₋₆alkyl or Het-C₀₋₆alkyl. Preferably R⁵ is selected from the group consisting of: C₁₋₆alkyl, C₃₋₆cycloalkyl-C₀₋₆alkyl, Ar—C₀₋₆alkyl and Het-C₀₋₆alkyl. More preferably R⁵ is selected from the group consisting of:

furanyl, especially furan-2-yl and furan-3-yl, more especially aryl substituted furanyl, even more especially 5-(4-chloro-phenyl)-furan-2-yl and 5-(3-trifluoromethyl-phenyl)-furan-2-yl;

benzofuranyl, especially benzofuran-2-yl, more especially C₁₋₆alkoxy substituted benzofuranyl, particularly 5,6-dimethoxy-benzofuran-2-yl and 5-(2-morpholin-4-yl-ethoxy)benzofuran-2-yl;

thiophenyl, especially thiophene-3-yl and thiophene-2-yl, more especially Het-C₀₋₆alkyl-thiophenyl; particularly 5-pyridin-2-yl-thiophene-2-yl, more especially C₁₋₆alkyl-thiophenyl, particularly 5-methyl-thiophene-2-yl and 3-methyl-thiophene-2-yl; more especially C₁₋₆alkoxy-thiophenyl, particularly 3-ethoxy-thiophene-2-yl;

furo[3,2-b]-pyridine-2-yl, especially C₁₋₆alkyl-furo[3,2-b]-pyridine-2-yl, more especially 3-methyl-furo[3,2-b]-pyridine-2-yl;

thiazolyl, especially thiazole-5-yl, more especially Het-C₀₋₆alkyl-thiazolyl, particularly 4-methyl-2-pyridin-2-yl-thiazole-5-yl;

phenyl, especially halogen substituted phenyl, particularly bromophenyl, more particularly 4-bromophenyl;

cyclobutyl;

cyclopentyl;

tetrahydrofuranyl, tetrahydrofuran-2-yl;

selenophenyl, especially selenophene-2-yl; and

thieno[3,2-b]thiophenyl, especially thieno[3,2-b]thiophene-2-yl.

R′ is selected from the group consisting of: H, C₁₋₆alkyl, Ar—C₀₋₆alkyl, and Het-C₀₋₆alkyl, preferably H.

R″ selected from the group consisting of: H, C₁₋₆alkyl, Ar—C₀₋₆alkyl, and Het-C₀₋₆alkyl, preferably H.

In compounds of Formula I, R² is selected from the group consisting of: H, C₁₋₆alkyl, C₃₋₆cycloalkyl-C₀₋₆alkyl, Ar—C₀₋₆alkyl, Het-C₀₋₆alkyl, R⁹C(O)—, R⁹C(S)—, R⁹SO₂—, R⁹OC(O)—, R⁹R¹¹NC(O)—, R⁹R¹¹NC(S)—, R⁹R¹¹NSO₂—,

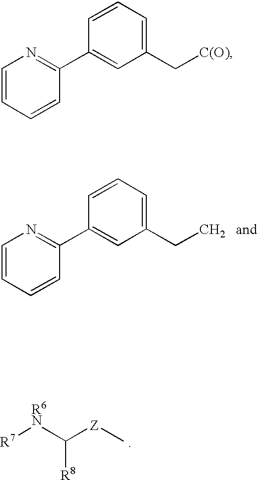

Preferably R² is selected from the group consisting of: R⁹SO₂ and C₁₋₆alkyl. When R² is C₁₋₆alkyl, C₁₋₆alkyl is preferably propyl. R² is most preferably R⁹SO₂.

R⁹ is selected from the group consisting of: C₁₋₆alkyl, C₃₋₆cycloalkyl-C₀₋₆alkyl, Ar—C₀₋₆alkyl, and Het-C₀₋₆alkyl, preferably Het-C₀₋₆alkyl, more preferably pyridinyl and 1-oxy-pyridinyl. When R² is R⁹SO₂, R⁹ is even more preferably selected from the group consisting of: pyridin-2-yl and 1-oxy-pyridin-2-yl. Most preferably, R⁹ is pyridin-2-yl.

More preferred are compounds of Formula I wherein:

R¹ is

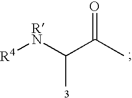

R² is R⁹SO₂;

R³ is C₃₋₆cycloalkyl-C₀₋₆alkyl;

R⁴ is R⁵C(O);

R⁵ is Het-C₀₋₆alkyl;

R⁹ is Het-C₀₋₆alkyl;

R′ is H

R″ is H; and

R′″ is C₁₋₆alkyl.

Even more preferred are compounds of Formula I wherein:

R¹ is

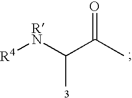

R² is R⁹SO₂;

R³ is cyclohexylmethyl;

R⁴ is R⁵C(O);

R⁵ is selected from the group consisting of: furanyl, especially furan-2-yl, and thiophenyl, especially thiophene-3-yl;

R⁹ is selected from the group consisting of: pyridin-2-yl and 1-oxy-pyridin-2-yl, preferably pyridin-2-yl;

R′ is H

R″ is H; and

R′″ is selected from the group consisting of: H and C₁₋₆alkyl. When R′″ is C₁₋₆alkyl, R′″ is:

especially selected from the group consisting of: methyl, ethyl, propyl, butyl, pentyl and hexyl, more especially methyl;

preferably selected from the group consisting of: 5-, 6- or 7-C₁₋₆alkyl, especially selected from the group consisting of: 5-, 6- or 7-methyl, -ethyl, -propyl, -butyl, -pentyl and -hexyl, more especially selected from the group consisting of: 5-, 6- or 7-methyl;

more preferably selected from the group consisting of: 6- or 7-C₁₋₆alkyl, especially selected from the group consisting of: 6- or 7-methyl, -ethyl, -propyl, -butyl, -pentyl and -hexyl, more especially selected from the group consisting of: 6- or 7-methyl;

yet more preferably cis-7-C₁₋₆alkyl as shown in Formula Ia:

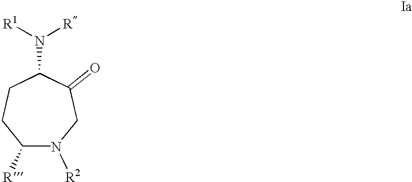

wherein R′″ is C₁₋₆alkyl, especially selected from the group consisting of: methyl, ethyl, propyl, butyl, pentyl and hexyl;

most preferably cis-7-methyl, as shown in Formula Ia wherein R′″ is methyl.

Compounds of Formula I selected from the following group are particularly preferred for use in the present invention:

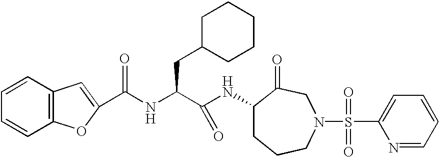

benzofuran-2-carboxylic acid {(S)-2-cyclohexyl-1-[3-oxo-1-(pyridine-2-sulfonyl)-azepan-4-ylcarbamoyl]-ethyl}-amide;

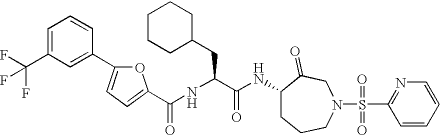

5-(3-trifluoromethyl-phenyl)-furan-2-carboxylic acid {(S)-2-cyclohexyl-1-[3-oxo-1-(pyridine-2-sulfonyl)-azepan-4-ylcarbamoyl]-ethyl}-amide;

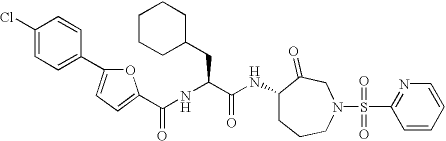

5-(4-chloro-phenyl)-furan-2-carboxylic acid {(S)-2-cyclohexyl-1-[3-oxo-1-(pyridine-2-sulfonyl)-azepan-4-ylcarbamoyl]-ethyl}-amide;

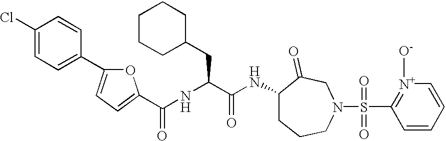

5-(4-chloro-phenyl)-furan-2-carboxylic acid {(S)-2-cyclohexyl-1-[3-oxo-1-(1-oxy-pyridine-2-sulfonyl)-azepan-4-ylcarbamoyl]-ethyl}-amide;

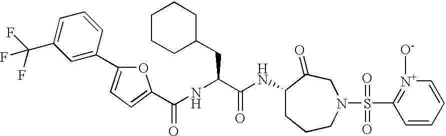

5-(3-trifluoromethyl-phenyl)-furan-2-carboxylic acid {(S)-2-cyclohexyl-1-[3-oxo-1-(1-oxy-pyridine-2-sulfonyl)-azepan-4-ylcarbamoyl]-ethyl}-amide;

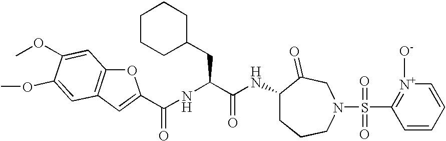

5,6-dimethoxy-benzofuran-2-carboxylic acid {(S)-2-cyclohexyl-1-[3-oxo-1-(1-oxy-pyridine-2-sulfonyl)-azepan-4-ylcarbamoyl]-ethyl}-amide;

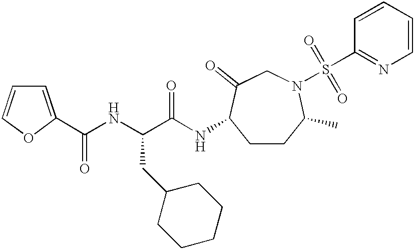

furan-2-carboxylic acid {(S)-2-cyclohexyl-1-[(4S,7R)-7-methyl-3-oxo-1-(pyridine-2sulfonyl)-azepan-4-ylcarbamoyl]-ethyl}-amide;

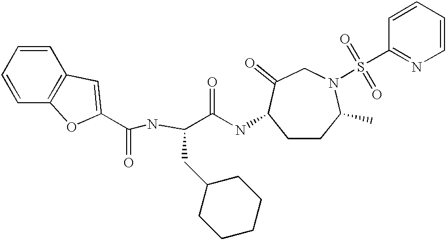

benzofuran-2-carboxylic acid {(S)-2-cyclohexyl-1-[(4S,7R)-7-methyl-3-oxo-1-(pyridine-2-sulfonyl)-azepan-4-ylcarbamoyl]-ethyl}-amide;

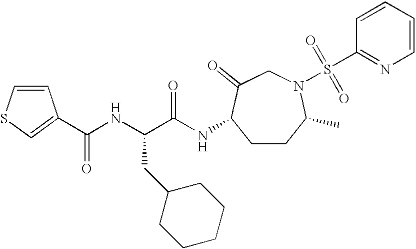

thiophene-3-carboxylic acid {(S)-2-cyclohexyl-1-[(4S,7R)-7-methyl-3-oxo-1-(pyridine-2-sulfonyl)-azepan-4-ylcarbamoyl]-ethyl}-amide;

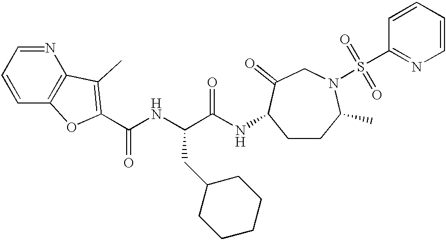

3-methyl-furo[3,2-b]-pyridine-2-carboxylic acid {(S)-2-cyclohexyl-1-[(4S,7R)-7-methyl-3-oxo-1-(pyridine-2-sulfonyl)-azepan-4-ylcarbamoyl]-ethyl}-amide;

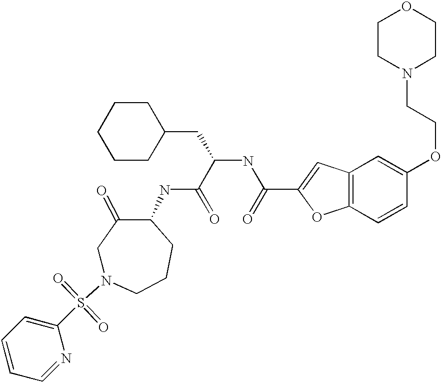

5-(2-morpholin-4-yl-ethoxy)-benzofuran-2-carboxylic acid {(S)-2-cyclohexyl-1-[3-oxo-1-(pyridine-2-sulfonyl)-azepan-4-ylcarbamoyl]-ethyl}-amide;

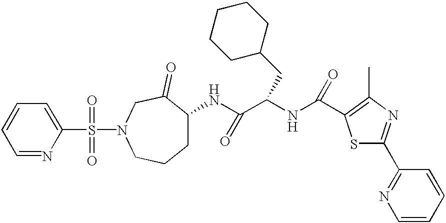

4-methyl-2-pyridin-2-yl-thiazole-5-carboxylic acid {(S)-2-cyclohexyl-1-[3-oxo-1-(pyridine-2-sulfonyl)-azepan-4-ylcarbamoyl]-ethyl}-amide;

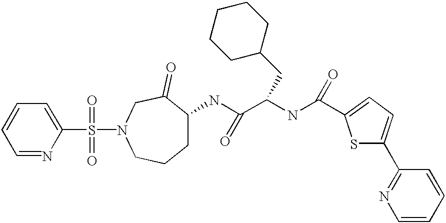

5-pyridin-2-yl-thiophene-2-carboxylic acid {(S)-2-cyclohexyl-1-[3-oxo-1-(pyridine-2-sulfonyl)-azepan-4-ylcarbamoyl]-ethyl}-amide;

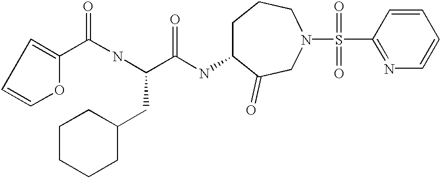

furan-2-carboxylic acid {(S)-2-cyclohexyl-1-[3-oxo-1-(pyridine-2-sulfonyl)-azepan-4-ylcarbamoyl]-ethyl}-amide;

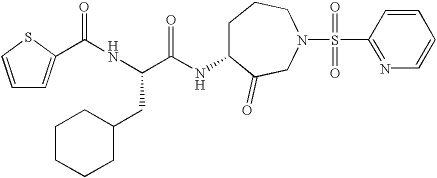

thiophene-2-carboxylic acid {(S)-2-cyclohexyl-1-[3-oxo-1-(pyridine-2-sulfonyl)-azepan-4-ylcarbamoyl]-ethyl}-amide;

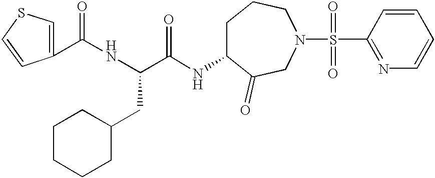

thiophene-3-carboxylic acid {(S)-2-cyclohexyl-1-[3-oxo-1-(pyridine-2-sulfonyl)-azepan-4-ylcarbamoyl]-ethyl}-amide;

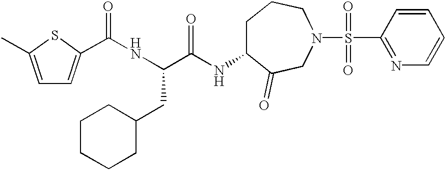

5-methyl-thiophene-2-carboxylic acid {(S)-2-cyclohexyl-1-[3-oxo-1-(pyridine-2-sulfonyl)-azepan-4-ylcarbamoyl]-ethyl}-amide;

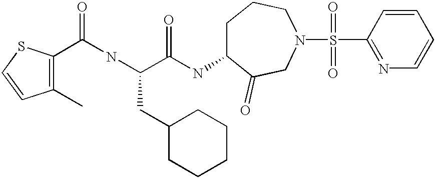

3-methyl-thiophene-2-carboxylic acid {(S)-2-cyclohexyl-1-[3-oxo-1-(pyridine-2-sulfonyl)-azepan-4-ylcarbamoyl]-ethyl}-amide;

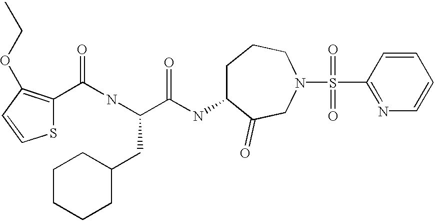

3-ethoxy-thiophene-2-carboxylic acid {(S)-2-cyclohexyl-1-[3-oxo-1-(pyridine-2-sulfonyl)-azepan-4-ylcarbamoyl]-ethyl}-amide;

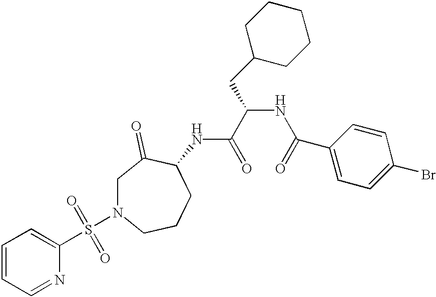

4-bromo-N-{(S)-2-cyclohexyl-1-[3-oxo-1-(pyridine-2-sulfonyl)-azepan-4-ylcarbamoyl]-ethyl}-benzamide;

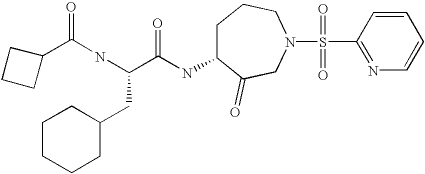

cyclobutanecarboxylic acid {(S)-2-cyclohexyl-1-[3-oxo-1-(pyridine-2-sulfonyl)-azepan-4-ylcarbarnoyl]-ethyl}-amide;

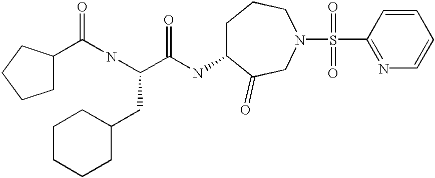

cyclopentanecarboxylic acid {(S)-2-cyclohexyl-1-[3-oxo-1-(pyridine-2-sulfonyl)-azepan-4-ylcarbamoyl]-ethyl}-amide;

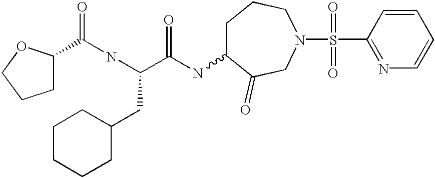

(S)-tetrahydro-furan-2-carboxylic acid {(S)-2-cyclohexyl-1-[3-oxo-1-(pyridine-2-sulfonyl)-azepan-4-ylcarbamoyl]-ethyl}-amide;

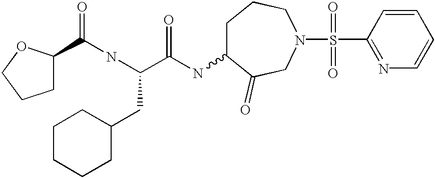

(R)-tetrahydro-furan-2-carboxylic acid {(S)-2-cyclohexyl-1-[3-oxo-1-(pyridine-2-sulfonyl)-azepan-4-ylcarbamoyl]-ethyl}-amide;

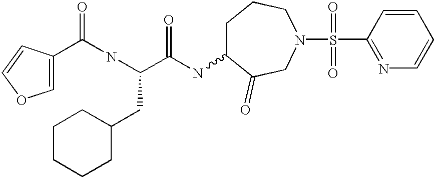

furan-3-carboxylic acid {(S)-2-cyclohexyl-1-[3-oxo-1-(pyridine-2-sulfonyl)-azepan-4-ylcarbamoyl]-ethyl}-amide;

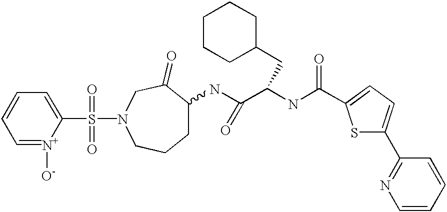

5-pyridin-2-yl-thiophene-2-carboxylic acid {(S)-2-cyclohexyl-1-[3-oxo-1-(1-oxy-pyridine-2-sulfonyl)-azepan-4-ylcarbamoyl]-ethyl}-amide;

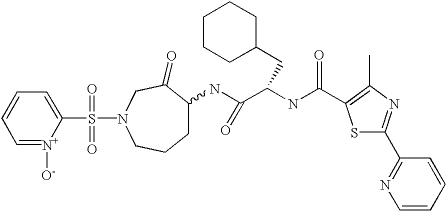

4-methyl-2-pyridin-2-yl-thiazole-5-carboxylic acid {(S)-2-cyclohexyl-1-[3-oxo-1-(1-oxy-pyridine-2-sulfonyl)-azepan-4-ylcarbamoyl]-ethyl}-amide;

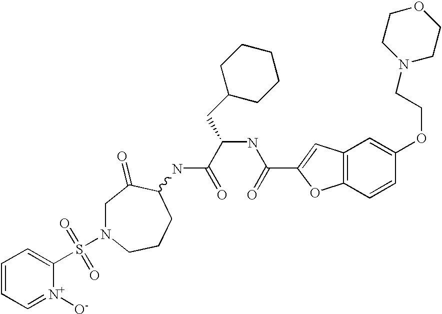

5-(2-morpholin-4-yl-ethoxy)-benzofuran-2-carboxylic acid {(S)-2-cyclohexyl-1-[3-oxo-1-(1-oxy-pyridine-2-sulfonyl)-azepan-4-ylcarbamoyl]-ethyl}-amide;

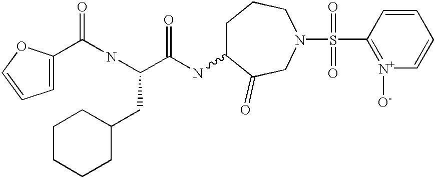

furan-2-carboxylic acid {(S)-2-cyclohexyl-1-[3-oxo-1-(1-oxy-pyridine-2-sulfonyl)-azepan-4-ylcarbamoyl]-ethyl}-amide;

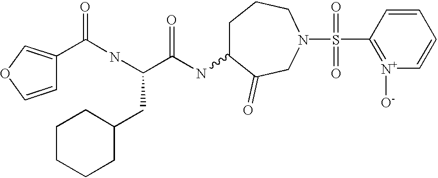

furan-3-carboxylic acid {(S)-2-cyclohexyl-1-[3-oxo-1-(1-oxy-pyridine-2-sulfonyl)-azepan-4-ylcarbamoyl]-ethyl}-amide;

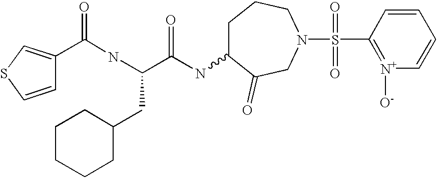

thiophene-3-carboxylic acid {(S)-2-cyclohexyl-1-[3-oxo-1-(1-oxy-pyridine-2-sulfonyl)-azepan-4-ylcarbamoyl]-ethyl}-amide;

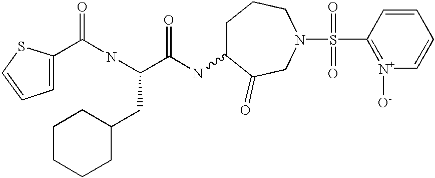

thiophene-2-carboxylic acid {(S)-2-cyclohexyl-1-[3-oxo-1-(1-oxy-pyridine-2-sulfonyl)-azepan-4-ylcarbamoyl]-ethyl}-amide;

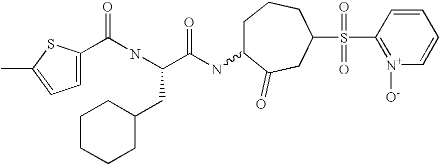

5-methyl-thiophene-2-carboxylic acid {(S)-2-cyclohexyl-1-[3-oxo-1-(1-oxy-pyridine-2-sulfonyl)-azepan-4-ylcarbamoyl]-ethyl}-amide;

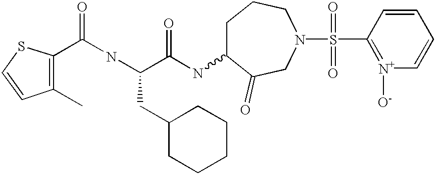

3-methyl-thiophene-2-carboxylic acid {(S)-2-cyclohexyl-1-[3-oxo-1-(1-oxy-pyridine-2-sulfonyl)-azepan-4-ylcarbamoyl]-ethyl}-amide;

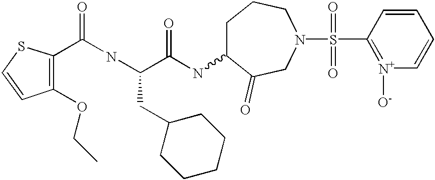

3-ethoxy-thiophene-2-carboxylic acid {(S)-2-cyclohexyl-1-[3-oxo-1-(1-oxy-pyridine-2-sulfonyl)-azepan-4-ylcarbamoyl]-ethyl}-amide;

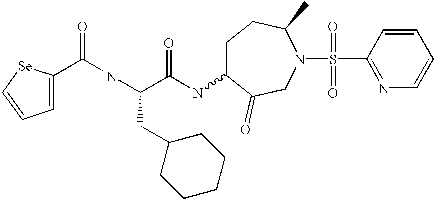

selenophene-2-carboxylic acid {(S)-2-cyclohexyl-1-[(R)-7-methyl-3-oxo-1-(pyridine-2-sulfonyl)-azepan-4-ylcarbamoyl]-ethyl}-amide;

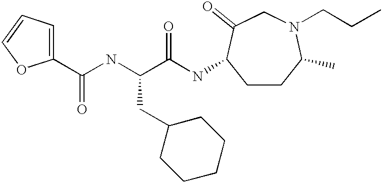

furan-2-carboxylic acid [(S)-2-cyclohexyl-1-((4S,7R)-7-methyl-3-oxo-1-propyl-azepan-4-ylcarbamoyl)-ethyl]-amide;

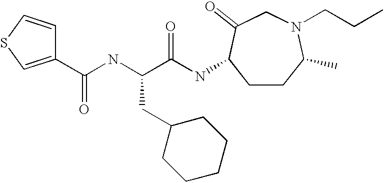

thiophene-3-carboxylic acid [(S)-2-cyclohexyl-1-((4S,7R)-7-methyl-3-oxo-1-propyl-azepan-4-ylcarbarnoyl)-ethyl]-amide;

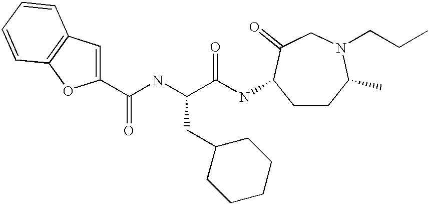

benzofuran-2-carboxylic acid [(S)-2-cyclohexyl-1-((4S,7R)-7-methyl-3-oxo-1-propyl-azepan-4-ylcarbamoyl)-ethyl]-amide;

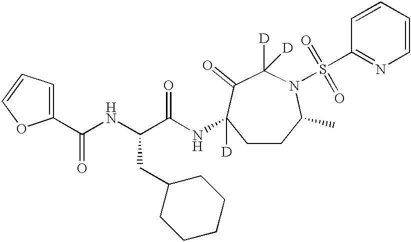

2,2,4-trideutero-Furan-2-carboxylic acid {(S)-2-cyclohexyl-1-[(4S,7R)-7-methyl-3-oxo-1-(pyridine-2-sulfonyl)-azepan-4-ylcarbamoyl]-ethyl}-amide;

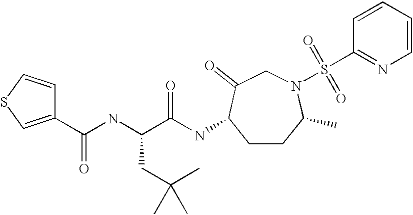

thiophene-3-carboxylic acid {(S)-3,3-dimethyl-1-[(4S,7R)-7-methyl-3-oxo-1-(pyridine-2-sulfonyl)-azepan-4-ylcarbamoyl]-butyl}-amide;

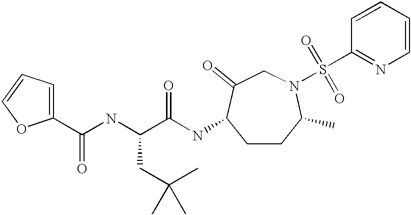

furan-2-carboxylic acid {(S)-3,3-dimethyl-1-[(4S,7R)-7-methyl-3-oxo-1-(pyridine-2-sulfonyl)-azepan-4-ylcarbamoyl]-butyl}-amide; and

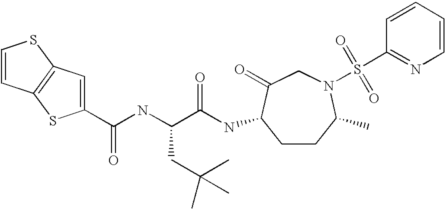

thieno[3,2-b]thiophene-2-carboxylic acid {(S)-3,3-dimethyl-1-[(4S,7R)-7-methyl-3-oxo-1-(pyridine-2-sulfonyl)-azepan-4-ylcarbamoyl]-butyl}-amide.

Compounds of Formula I selected from the following group are more particularly preferred for use in the present invention:

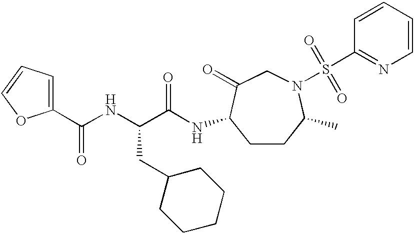

furan-2-carboxylic acid {(S)-2-cyclohexyl-1-[(4S,7R)-7-methyl-3-oxo-1-(pyridine-2-sulfonyl)-azepan-4-ylcarbamoyl]-ethyl}-amide; and

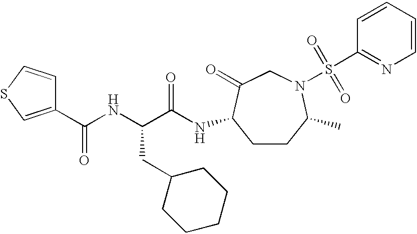

thiophene-3-carboxylic acid {(S)-2-cyclohexyl-1-[(4S,7R)-7-methyl-3-oxo-1-(pyridine-2-sulfonyl)-azepan-4-ylcarbamoyl]-ethyl}-amide.

The following compound of Formula I is the most preferred for use in the present invention:

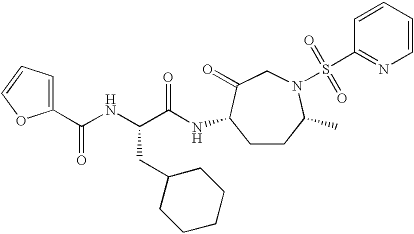

furan-2-carboxylic acid {(S)-2-cyclohexyl-1-[(4S,7R)-7-methyl-3-oxo-1-(pyridine-2-sulfonyl)-azepan-4-ylcarbamoyl]-ethyl}-amide.

The present invention provides the following novel compounds:

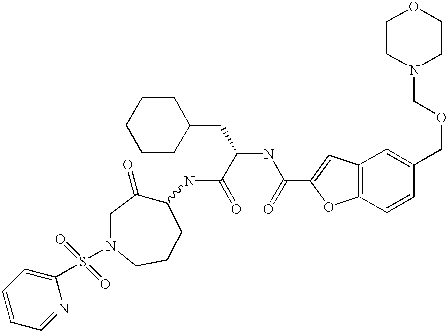

5-(2-morpholin-4yl-ethoxy)-benzofuran-2-carboxylic acid {(S)-2-cyclohexyl-1-[3-oxo-1-(pyridine-2-sulfonyl)-azepan-4-ylcarbamoyl]-ethyl}-amide;

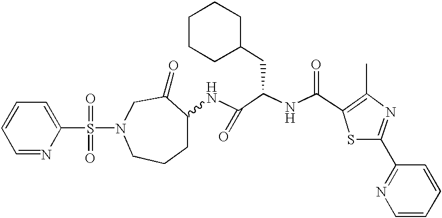

4-methyl-2-pyridin-2-yl-thiazole-5-carboxylic acid {(S)-2-cyclohexyl-1-[3-oxo-1-(pyridine-2-sulfonyl)-azepan-4-ylcarbamoyl]-ethyl}-amide;

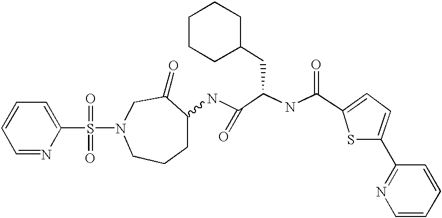

5-pyridin-2-yl-thiophene-2-carboxylic acid {(S)-2-cyclohexyl-1-[3-oxo-1-(pyridine-2-sulfonyl)-azepan-4-ylcarbamoyl]-ethyl}-amide;

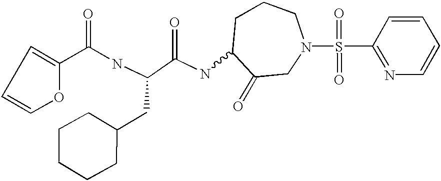

furan-2-carboxylic acid {(S)-2-cyclohexyl-1-[3-oxo-1-(pyridine-2-sulfonyl)-azepan-4-ylcarbamoyl]-ethyl}-amide;

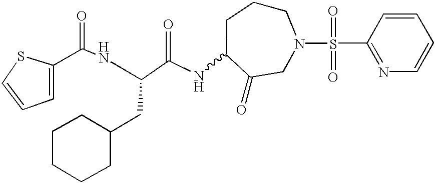

thiophene-2-carboxylic acid {(S)-2-cyclohexyl-1-[3-oxo-1-(pyridine-2-sulfonyl)-azepan-4-ylcarbamoyl]-ethyl}-amide;

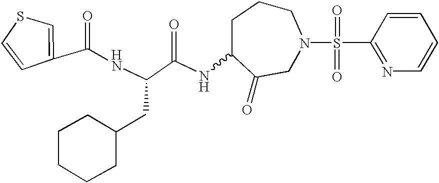

thiophene-3-carboxylic acid {(S)-2-cyclohexyl-1-[3-oxo-1-(pyridine-2-sulfonyl)-azepan-4-ylcarbamoyl]-ethyl}-amide;

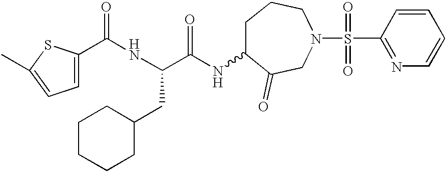

5-methyl-thiophene-2-carboxylic acid {(S)-2-cyclohexyl-1-[3-oxo-1-(pyridine-2-sulfonyl)-azepan-4-ylcarbamoyl]-ethyl}-amide;

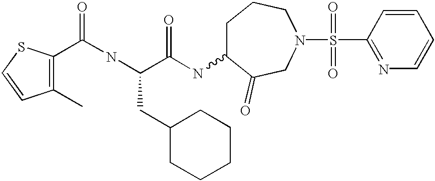

3-methyl-thiophene-2-carboxylic acid {(S)-2-cyclohexyl-1-[3-oxo-1-(pyridine-2-sulfonyl)-azepan-4-ylcarbamoyl]-ethyl}-amide;

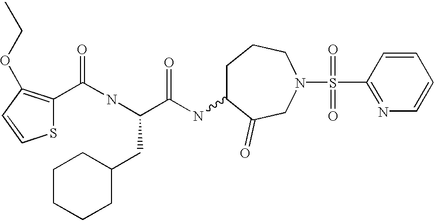

3-ethoxy-thiophene-2-carboxylic acid {(S)-2-cyclohexyl-1-[3-oxo-1-(pyridine-2-sulfonyl)-azepan-4-ylcarbamoyl]-ethyl}-amide;

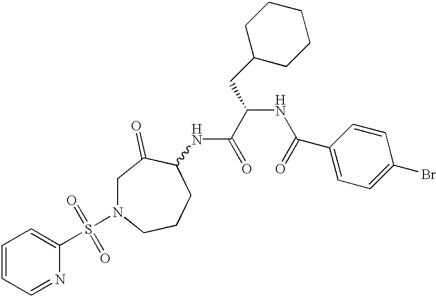

4-bromo-N-{(S)-2-cyclobexyl-1-[3-oxo-1-(pyridine-2-sulfonyl)-azepan-4-ylcarbamoyl]-ethyl}-benzamide;

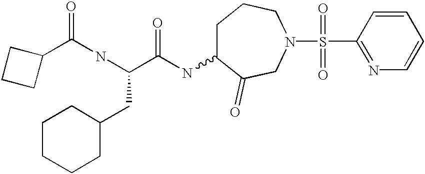

cyclobutanecarboxylic acid {(S)-2-cyclohexyl-1-[3-oxo-1-(pyridine-2-sulfonyl)-azepan-4-ylcarbamoyl]-ethyl}-amide;

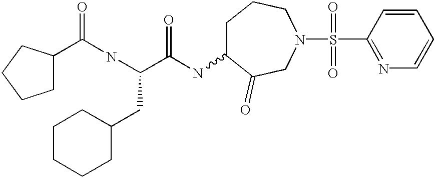

cyclopentanecarboxylic acid {(S)-2-cyclohexyl-1-[3-oxo-1-(pyridine-2-sulfonyl)-azepan-4-ylcarbamoyl]-ethyl}-amide;

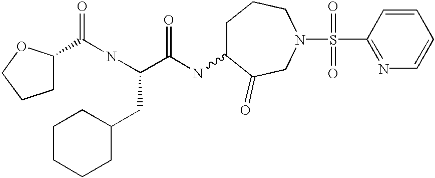

(S)-tetrahydro-furan-2-carboxylic acid {(S)-2-cyclohexyl-1-[3-oxo-1-(pyridine-2-sulfonyl)-azepan-4-ylcarbamoyl]-ethyl}-amide;

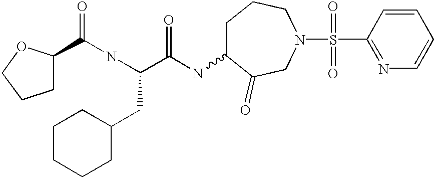

(R)-tetrahydro-furan-2-carboxylic acid {(S)-2-cyclohexyl-1-[3-oxo-1-(pyridine-2-sulfonyl)-azepan-4-ylcarbamoyl]-ethyl}-amide;

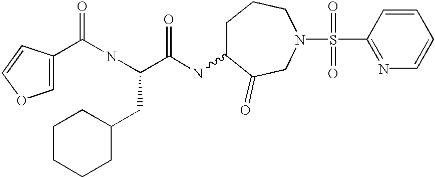

furan-3-carboxylic acid {(S)-2-cyclohexyl-1-[3-oxo-1-(pyridine-2-sulfonyl)-azepan-4-ylcarbamoyl]-ethyl}-amide;

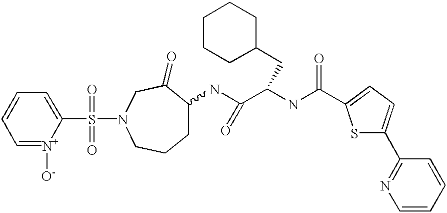

5-pyridin-2-yl-thiophene-2-carboxylic acid {(S)-2-cyclohexyl-1-[3-oxo-1-(1-oxy-pyridine-2-sulfonyl)-azepan-4-ylcarbamoyl]-ethyl}-amide;

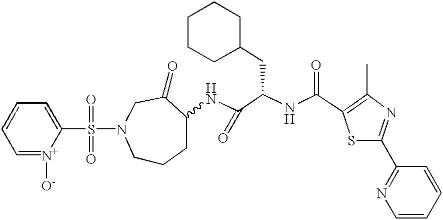

4-methyl-2-pyridin-2-yl-thiazole-5-carboxylic acid {(S)-2-cyclohexyl-1-[3-oxo-1-(1-oxy-pyridine-2-sulfonyl)-azepan-4-ylcarbamoyl]-ethyl}-amide;

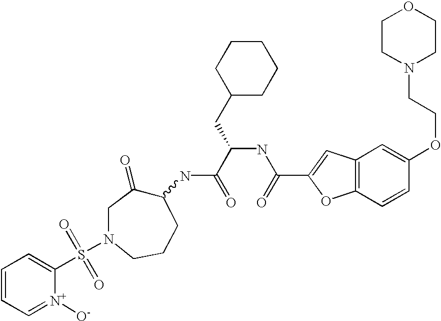

5-(2-morpholin-4-yl-ethoxy)-benzofuran-2-carboxylic acid {(S)-2-cyclohexyl-1-[3-oxo-1-(1-oxy-pyridine-2-sulfonyl)-azepan-4-ylcarbamoyl]-ethyl}-amide;

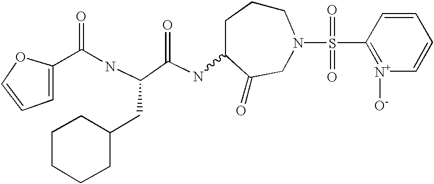

furan-2-carboxylic acid {(S)-2-cyclohexyl-1-[3-oxo-1-(1-oxy-pyridine-2-sulfonyl)-azepan-4-ylcarbamoyl]-ethyl}-amide;

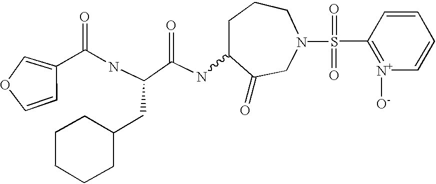

furan-3-carboxylic acid {(S)-2-cyclohexyl-1-[3-oxo-1-(1-oxy-pyridine-2-sulfonyl)-azepan-4-ylcarbamoyl]-ethyl}-amide;

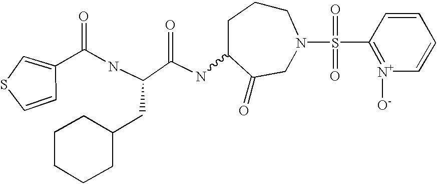

thiophene-3-carboxylic acid {(S)-2-cyclohexyl-1-[3-oxo-1-(1-oxy-pyridine-2-sulfonyl)-azepan-4-ylcarbamoyl]-ethyl}-amide;

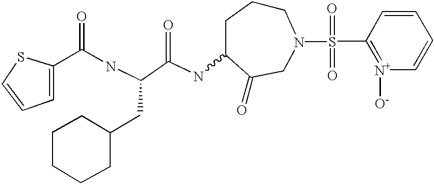

thiophene-2-carboxylic acid {(S)-2-cyclohexyl-1-[3-oxo-1-(1-oxy-pyridine-2-sulfonyl)-azepan-4-ylcarbamoyl]-ethyl}-amide;

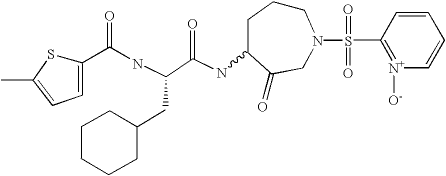

5-methyl-thiophene-2-carboxylic acid {(S)-2-cyclohexyl-1-[3-oxo-1-(1-oxy-pyridine-2-sulfonyl)-azepan-4-ylcarbamoyl]-ethyl}-amide;

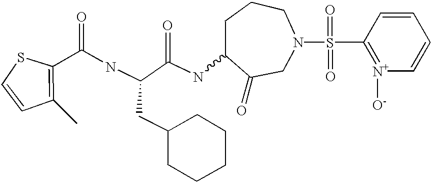

3-methyl-thiophene-2-carboxylic acid {(S)-2-cyclohexyl-1-[3-oxo-1-(1-oxy-pyridine-2-sulfonyl)-azepan-4-ylcarbamoyl]-ethyl}-amide;

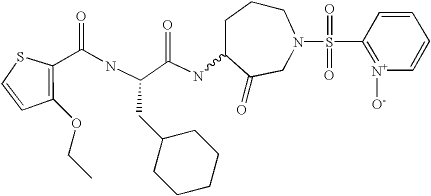

3-ethoxy-thiophene-2-carboxylic acid {(S)-2-cyclohexyl-1-[3-oxo-1-(1-oxy-pyridine-2-sulfonyl)-azepan-4-ylcarbamoyl]-ethyl}-amide;

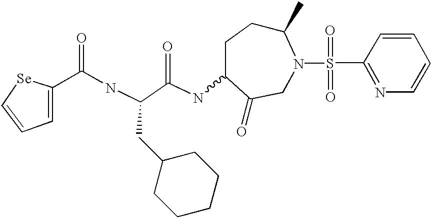

selenophene-2-carboxylic acid {(S)-2-cyclohexyl-1-[(R)-7-methyl-3-oxo-1-(pyridine-2-sulfonyl)-azepan-4-ylcarbamoyl]-ethyl}-amide; and

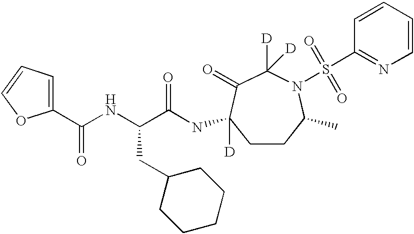

2,2,4-trideutero-Furan-2-carboxylic acid {(S)-2-cyclohexyl-1-[(4S,7R)-7-methyl-3-oxo-1-(pyridine-2-sulfonyl)-azepan-4-ylcarbamoyl]-ethyl}-amide.

Specific representative compounds used in the present invention are set forth in Examples 1-44.

Compared to the corresponding 5 and 6 membered ring compounds, the 7 membered ring compounds used in the present invention are configurationally more stable at the carbon center alpha to the ketone.

The present invention also uses deuterated analogs of the inventive compounds. Representative examples of such deuterated compounds are set forth in Examples 7 and 41. A representative synthetic route for the deuterated compounds of the present invention are set forth in Scheme 3 and Examples 7 and 41, below. The deuterated compounds used in the present invention exhibit superior chiral stability compared to the protonated isomer.

### Definitions

The compounds used in the present invention include all hydrates, solvates, complexes and prodrugs. Prodrugs are any covalently bonded compounds which release the active parent drug according to Formula I in vivo. If a chiral center or another form of an isomeric center is present in a compound used in the present invention, all forms of such isomer or isomers, including enantiomers and diastereomers, are intended to be covered herein. Compounds used in the present methods containing a chiral center may be used as a racemic mixture, an enantiomerically enriched mixture, or the racemic mixture may be separated using well-known techniques and an individual enantiomer may be used alone. In cases in which compounds have unsaturated carbon-carbon double bonds, both the cis (Z) and trans (E) isomers are within the scope of this invention. In cases wherein compounds may exist in tautomeric forms, such as keto-enol tautomers, each tautomeric form is contemplated as being included within this invention whether existing in equilibrium or predominantly in one form.

The meaning of any substituent at any one occurrence in Formula I or any subformula thereof is independent of its meaning, or any other substituent's meaning, at any other occurrence, unless specified otherwise.

Abbreviations and symbols commonly used in the peptide and chemical arts are used herein to describe the compounds of the present invention. In general, the amino acid abbreviations follow the IUPAC-IUB Joint Commission on Biochemical Nomenclature as described in Eur. J. Biochem., 158, 9 (1984).

“Proteases” are enzymes that catalyze the cleavage of amide bonds of peptides and proteins by nucleophilic substitution at the amide bond, ultimately resulting in hydrolysis. Such proteases include: cysteine proteases, serine proteases, aspartic proteases, and metalloproteases. The compounds of the present invention are capable of binding more strongly to the enzyme than the substrate and in general are not subject to cleavage after enzyme catalyzed attack by the nucleophile. They therefore competitively prevent proteases from recognizing and hydrolyzing natural substrates and thereby act as inhibitors.

The term “amino acid” as used herein refers to the D- or L-isomers of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine.

“Hydrogen” or “H” includes all of its possible isotopes, including “deuterium” or “D” or “²H”; and “tritium” or “T” or “³H”.

“C₁₋₆alkyl” as applied herein is meant to include substituted and unsubstituted methyl, ethyl, n-propyl, isopropyl, n-butyl, isobutyl and t-butyl, pentyl, n-pentyl, isopentyl, neopentyl and hexyl and the simple aliphatic isomers thereof. C₁₋₆alkyl may be optionally substituted by a moiety selected from the group consisting of: OR¹², C(O)R¹², SR¹², S(O)R¹², NR¹²₂, R¹²NC(O)OR⁵, CO₂R¹², CO₂NR¹²₂, N(C═NH)NH₂, Het, C₃₋₆cycloalkyl, and Ar; where R⁵ is selected from the group consisting of: H, C₁₋₆alkyl, C₂₋₆alkenyl, C₂₋₆alkynyl, C₃₋₆cycloalkyl-C₀₋₆alkyl, Ar—C₀₋₆alkyl and Het-C₀₋₆alkyl; and R¹² is selected from the group consisting of: H, C₁₋₆alkyl, Ar—C₀₋₆alkyl, and Het-C₀₋₆alkyl;

“C₃₋₆cycloalkyl” as applied herein is meant to include substituted and unsubstituted cyclopropane, cyclobutane, cyclopentane and cyclohexane.

“C₂₋₆ alkenyl” as applied herein means an alkyl group of 2 to 6 carbons wherein a carbon-carbon single bond is replaced by a carbon-carbon double bond. C₂₋₆alkenyl includes ethylene, 1-propene, 2-propene, 1-butene, 2-butene, isobutene and the several isomeric pentenes and hexenes. Both cis and trans isomers are included.

“C₂₋₆alkynyl” means an alkyl group of 2 to 6 carbons wherein one carbon-carbon single bond is replaced by a carbon-carbon triple bond. C₂₋₆ alkynyl includes acetylene, 1-propyne, 2-propyne, 1-butyne, 2-butyne, 3-butyne and the simple isomers of pentyne and hexyne.

“Halogen” means F, Cl, Br, and I.

“Ar” or “aryl” means phenyl or naphthyl, optionally substituted by one or more of Ph-C₀₋₆alkyl; Het-C₀₋₆alkyl; C₁₋₆alkoxy; Ph-C₀₋₆alkoxy; Het-C₀₋₆alkoxy; OH, (CH₂)₁₋₆NR¹⁵R¹⁶; O(CH₂)₁₋₆NR¹⁵R¹⁶; C₁₋₆alkyl, OR¹⁷, N(R¹⁷)₂, SR¹⁷, CF₃, NO₂, CN, CO₂R¹⁷, CON(R¹⁷), F, Cl, Br or I; where R¹⁵ and R¹⁶ are H, C₁₋₆alkyl, Ph-C₀₋₆alkyl, naphthyl-C₀₋₆alkyl or Het-C₀₋₆alkyl; and R¹⁷ is phenyl, naphthyl, or C₁₋₆alkyl.

As used herein “Het” or “heterocyclic” represents a stable 5- to 7-membered monocyclic, a stable 7- to 10-membered bicyclic, or a stable 11- to 18-membered tricyclic heterocyclic ring which is either saturated or unsaturated, and which consists of carbon atoms and from one to three heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure, and may optionally be substituted with one or two moieties selected from C₀₋₆Ar, C₁₋₆alkyl, OR¹⁷, N(R¹⁷)₂, SR¹⁷, CF₃, NO₂, CN, CO₂R¹⁷, CON(R¹⁷), F, Cl, Br and I, where R¹⁷ is phenyl, naphthyl, or C₁₋₆alkyl. Examples of such heterocycles include piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, pyridinyl, 1-oxo-pyridinyl, pyrazinyl, oxazolidinyl, oxazolinyl, oxazolyl, isoxazolyl, morpholinyl, thiazolidinyl, thiazolinyl, thiazolyl, quinuclidinyl, indolyl, quinolinyl, quinoxalinyl, isoquinolinyl, benzimidazolyl, benzopyranyl, benzoxazolyl, furanyl, benzofuranyl, thiophenyl, benzo[b]thiophenyl, thieno[3,2-b]thiophenyl, benzo[1,3]dioxolyl, 1,8 naphthyridinyl, pyranyl, tetrahydrofuranyl, tetrahydropyranyl, thienyl, benzoxazolyl, thiamorpholinyl sulfoxide, thiamorpholinyl sulfone, and oxadiazolyl, as well as triazolyl, thiadiazolyl, oxadiazolyl, isothiazolyl, imidazolyl, pyridazinyl, pyrimidinyl, triazinyl and tetrazinyl which are available by routine chemical synthesis and are stable. The term heteroatom as applied herein refers to oxygen, nitrogen and sulfur.

Here and throughout this application the term C₀ denotes the absence of the substituent group immediately following; for instance, in the moiety ArC₀₋₆alkyl, when C is 0, the substituent is Ar, e.g., phenyl. Conversely, when the moiety ArC₀₋₆alkyl is identified as a specific aromatic group, e.g., phenyl, it is understood that the value of C is 0.

Certain radical groups are abbreviated herein. t-Bu refers to the tertiary butyl radical, Boc refers to the t-butyloxycarbonyl radical, Fmoc refers to the fluorenylmethoxycarbonyl radical, Ph refers to the phenyl radical, Cbz refers to the benzyloxycarbonyl radical.

Certain reagents are abbreviated herein. m-CPBA refers to 3-chloroperoxybenzoic acid, EDC refers to N-ethyl-N′(dimethylaminopropyl)-carbodiimide, P-EDC refers to polymer supported EDC, DMF refers to dimethyl formamide, DMSO refers to dimethyl sulfoxide, NMM is N-methylmorpholine, TEA refers to triethylamine, TFA refers to trifluoroacetic acid, and THF refers to tetrahydrofuran.

### Methods of Preparation

Compounds of the general formula I may be prepared in a fashion analogous to that outlined in Schemes 1 to 5. Alkylation of benzyl-N-allylcarbamate (1) with a base such as sodium hydride and 5-bromo-1-pentene provides the diene 2 (Scheme 1). Treatment of 2 bis(tricyclohexylphosphine)benzylidine ruthenium (IV) dichloride olefin metathesis catalysts developed by Grubbs provides the tetrahydroazepine 3. Epoxidation of 3 with oxidizing agents common to the art such as m-CPBA provides the epoxide 4. Nucleophilic epoxide ring opening may be effected with a reagent such as sodium azide to provide the azido alcohol 5 which may be reduced to the amino alcohol 6 under conditions common to the art such as 1,3-propanedithiol and triethylamine in methanol or triphenylphosphine in THF and water. The amine of compound 6 may be protected with with di-tert-butyldicarbonate to provide the N-Boc derivative 7 (Scheme 2). Removal of the benzyloxycarbonyl protecting group may be effected by treatment of 7 with hydrogen gas in the presence of a catalyst such as 10% Pd/C to provide the amine 8. Treatment of amine 8 with a sulfonyl chloride such as 2-pyridinesulfonyl chloride in the presence of a base such as N-methylmorpholine or triethylamine provides the sulfonamide derivative 9. Removal of the tert-butoxycarbonyl protecting group may be effected with an acid such as hydrochloric acid to provide intermediate 10. Coupling of 10 with an acid such as N-Boc-phenylalanine in the presence of a coupling agent common to the art such as HBTU or polymer supported EDC provides the alcohol intermediate 11. Removal of the tert-butoxycarbonyl protecting group under acidic conditions provides amine 12. Coupling of 12 with an acid such as benzofuran-2-carboxylic acid in the presence of a coupling agent such as HBTU or polymer supported EDC provides alcohol 13. Alcohol 13 may be oxidized with an oxidant common to the art such as pyridine sulfur trioxide complex in DMSO and triethylamine or the Dess-Martin periodinane to provide the ketone 14.

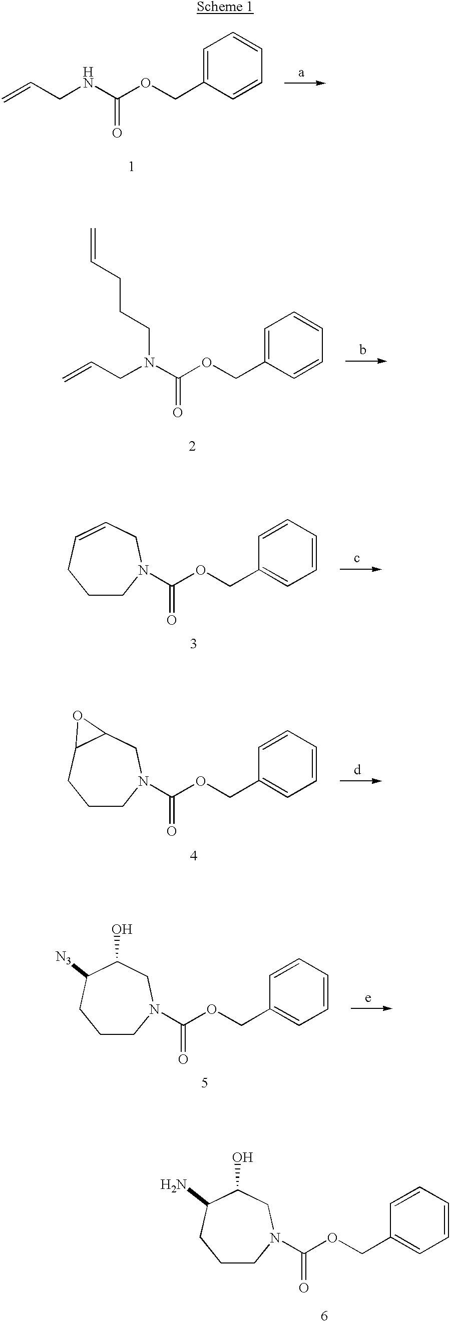

Reagents and conditions: (a) NaH, 5-bromo-1-pentene, NaH; (b) bis(tricyclohexylphosphine)benzylidine ruthenium (IV) dichloride, CH₂Cl₂, reflux; (c) m-CPBA, CH₂Cl₂; (d) NaN₃, NH₄Cl, CH₃OH, H₂O; (e) TEA, 1,3-propanedithiol, CH₃OH.

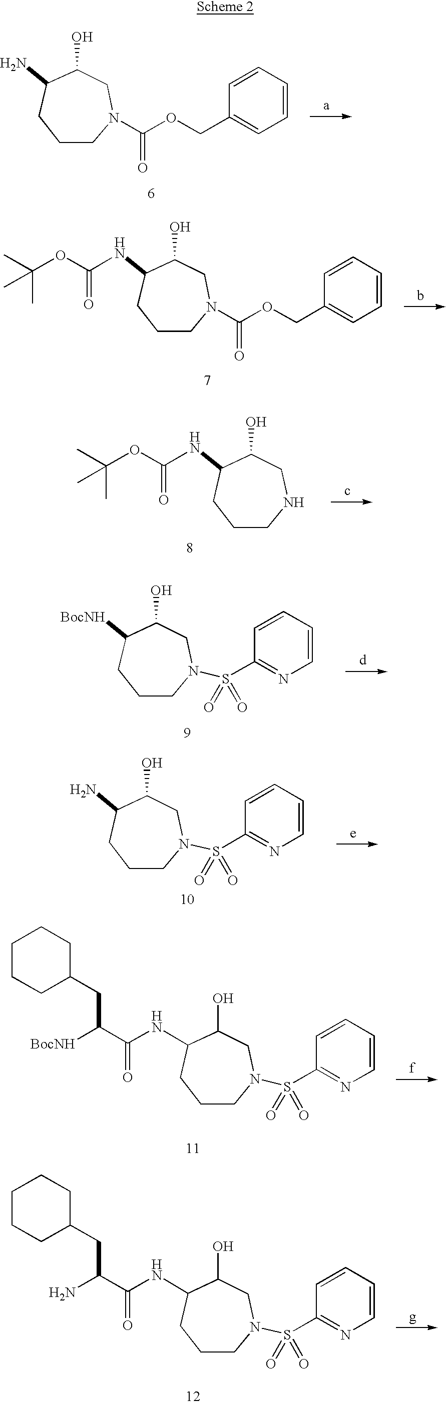

Reagents and conditions: (a) Di-tert-butyldicarbonate, THF; (b) H₂, 10% Pd/C, EtOAc; (c) 2-pyridylsulfonyl chloride, TEA, DMF; (d) HCl, EtOAc; (e) N-Boc-cylohexylalanine, P-EDC, CH₂Cl₂; (f) HCl, CH₂Cl₂; (g) benzofuran-2-carboxylic acid, P-EDC, CH₂Cl₂; (h) Dess-Martin periodinane, methylene chloride.

The deuterated compound of the Example 7 may be conveniently prepared according to Scheme 3. The skilled artisan will understand from Example 7 and Scheme 3 how to make any of the the deuterated compounds of the present invention.

The individual diastereomers of benzofuran-2-carboxylic acid {(S)-3-methyl-1-[(2,2′,4-trideuterio)-3-oxo-1-(pyridine-2-sulfonyl)-azepan-4-ylcarbamoyl]-butyl}amide 16 and 17 may be prepared as outlined in Scheme 3

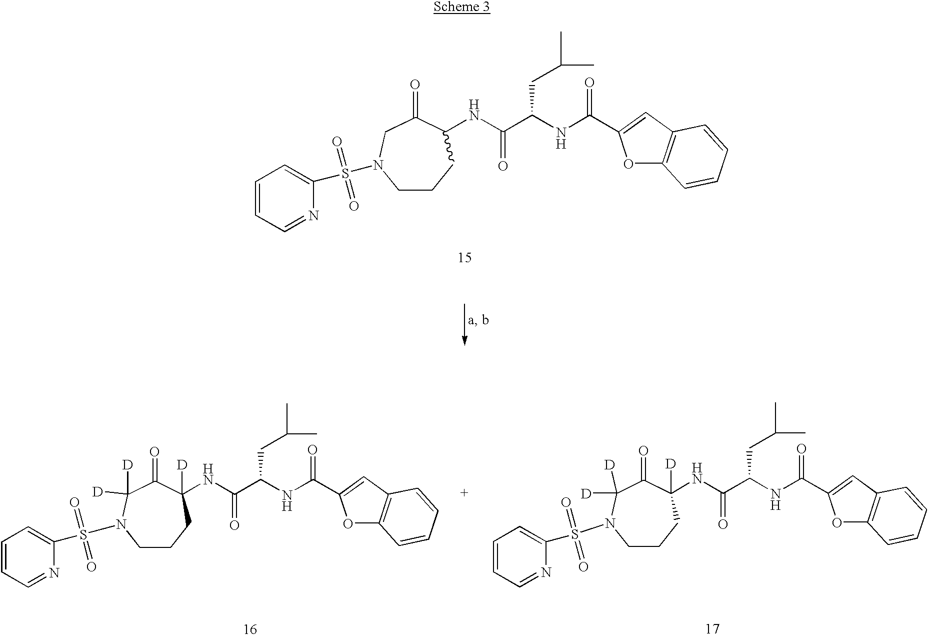

Reagents and Conditions: a.) CD₃OD;D₂O (10:1), TEA; b.) HPLC separation.

Treatment of the diastereomeric ketones 15 with triethylamine in CD₃OD:D₂O at reflux provides the deuterated analog as a mixture of diastereomers which are separated by HPLC to provide the deuterated compounds 16 and 17.

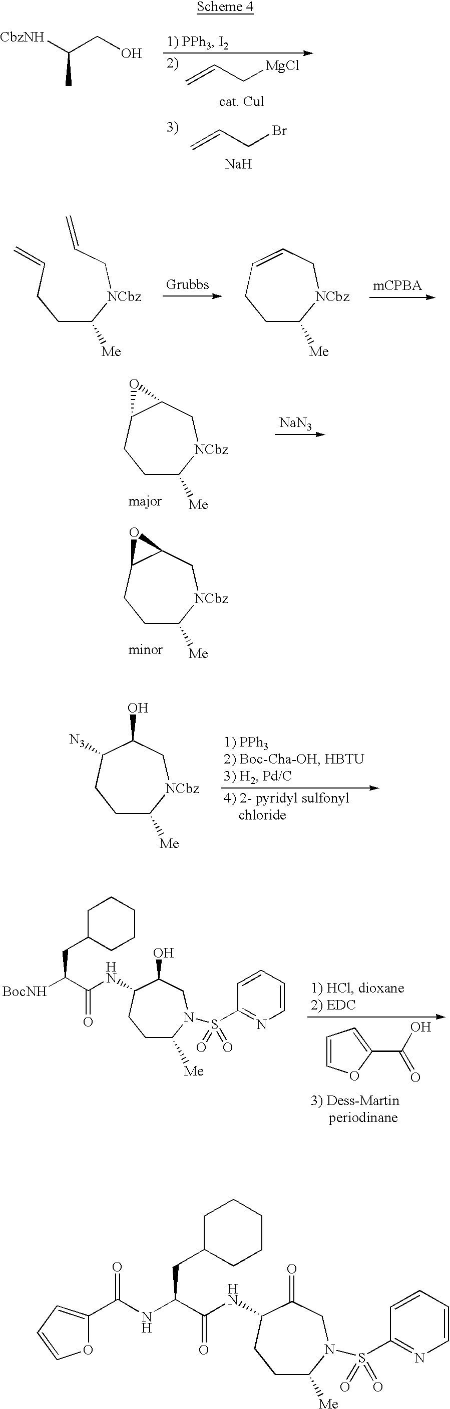

Scheme 4, carbobenyzloxy-D-alaninol (Cbz-D-alaninol)is first converted to an iodide, then is reacted with allyl Grignard with a copper (I) catalyst or a similar allyl organometallic reagent. The amine is then alkylated with allyl iodide. Grubbs' catalyst is then used to form the azapine ring by ring closing metathesis. Epoxidation of the alkene followed by separation of the diastereomers followed by opening of the epoxide of the minor component with sodium azide provides the intermediate azido alcohol. Reduction of the azide followed by acylation of the amine with a protected amino acid such as Boc-cyclohexylalanine, followed by deprotection of the Cbz gives the intermediate secondary amine, which is then sulfonylated with a sulfonyl chloride such as pyridine sulfonyl sulfonyl chloride. Deprotection of the Boc group followed by acylation with an acylating agent such as 2-furan carboxylic acid, HBTU, NMM, and final oxidation of the secondary alcohol to the ketone provides the desired products.

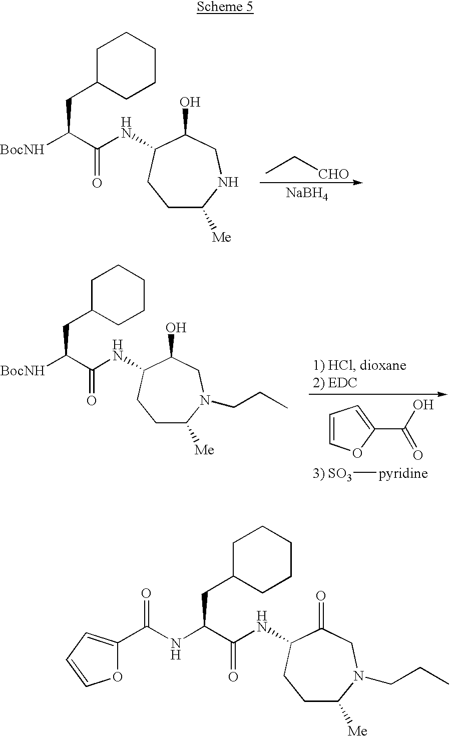

Intermediate (S)-3-Cyclohexyl-N-((3S,4S,7R)-3-hydroxy-7-methyl-azepan-4-yl)-2-methyl-propionamide, as described in Scheme 4, is reductively aminated with an aldehyde or a ketone such as propionaldehyde, then treated with a reducing agent such as sodium borohydride. Deprotection of the Boc group followed by acylation with an acylating agent such as 2-furan carboxylic acid, HBTU, NMM, and final oxidation of the secondary alcohol to the ketone provides the desired products.

The starting materials used herein are commercially available amino acids or are prepared by routine methods well known to those of ordinary skill in the art and can be found in standard reference books, such as the COMPENDIUM OF ORGANIC SYNTHETIC METHODS, Vol. I-VI (published by Wiley-Interscience).

Coupling methods to form amide bonds herein are generally well known to the art. The methods of peptide synthesis generally set forth by Bodansky et al., THE PRACTICE OF PEPTIDE SYNTHESIS, Springer-Verlag, Berlin, 1984; E. Gross and J. Meienhofer, THE PEPTIDES, Vol. 1, 1-284 (1979); and J. M. Stewart and J. D. Young, SOLID PHASE PEPTIDE SYNTHESIS, 2d Ed., Pierce Chemical Co., Rockford, Ill., 1984. are generally illustrative of the technique and are incorporated herein by reference.

Synthetic methods to prepare the compounds of this invention frequently employ protective groups to mask a reactive functionality or minimize unwanted side reactions. Such protective groups are described generally in Green, T. W, PROTECTIVE GROUPS IN ORGANIC SYNTHESIS, John Wiley & Sons, New York (1981). The term “amino protecting groups” generally refers to the Boc, acetyl, benzoyl, Fmoc and Cbz groups and derivatives thereof as known to the art. Methods for protection and deprotection, and replacement of an amino protecting group with another moiety are well known.

Acid addition salts of the compounds of Formula I are prepared in a standard manner in a suitable solvent from the parent compound and an excess of an acid, such as hydrochloric, hydrobromic, hydrofluoric, sulfuric, phosphoric, acetic, trifluoroacetic, maleic, succinic or methanesulfonic. Certain of the compounds form inner salts or zwitterions which may be acceptable. Cationic salts are prepared by treating the parent compound with an excess of an alkaline reagent, such as a hydroxide, carbonate or alkoxide, containing the appropriate cation; or with an appropriate organic amine. Cations such as Li⁺, Na⁺, K⁺, Ca⁺⁺, Mg⁺⁺ and NH₄⁺ are specific examples of cations present in pharmaceutically acceptable salts. Halides, sulfate, phosphate, alkanoates (such as acetate and trifluoroacetate), benzoates, and sulfonates (such as mesylate) are examples of anions present in pharmaceutically acceptable salts.

The methods of the present invention may be practiced by administering a pharmaceutical composition which comprises one or more compounds according to Formula I and a pharmaceutically acceptable carrier, diluent or excipient. Accordingly, the compounds of Formula I may be used in the manufacture of a medicament. Pharmaceutical compositions of the compounds of Formula I prepared as hereinbefore described may be formulated as solutions or lyophilized powders for parenteral administration. Powders may be reconstituted by addition of a suitable diluent or other pharmaceutically acceptable carrier prior to use. The liquid formulation may be a buffered, isotonic, aqueous solution. Examples of suitable diluents are normal isotonic saline solution, standard 5% dextrose in water or buffered sodium or amrnonium acetate solution. Such formulation is especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride or sodium citrate.

Alternately, these compounds may be encapsulated, tableted or prepared in an emulsion or syrup for oral administration. Pharmaceutically acceptable solid or liquid carriers may be added to enhance or stabilize the composition, or to facilitate preparation of the composition. Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

For rectal administration, the compounds of this invention may also be combined with excipients such as cocoa butter, glycerin, gelatin or polyethylene glycols and molded into a suppository.

### Utility of the Present Invention

The compounds of Formula I are useful as inhibitors of cathepsin S. The present invention provides methods of treatment of diseases caused by pathological levels of cathepsin S, which methods comprise administering to an animal, particularly a mammal, most particularly a human in need thereof a therapeutically effective amount of an inhibitor of cathepsin S, including one or more compounds of the present invention.

The present invention particularly provides methods for treating the following diseases in which cathepsin S is implicated:

treatment and/or prevention of an autoimmune disease state such as rheumatoid arthritis, multiple sclerosis, juvenile-onset diabetes, sytemic lupus erythematosus, discoid lupus erythematosus, pemphigus vulgaris, pemphigoid, Grave's disease, myasthenia gravis, Hashimoto's thyroiditis, scleroderma, dermatomysositis, Addison's disease, pernicious anemia, primary myxoedema, thyrotoxicosis, autoimmune atrophic gastritis, stiff-man syndrome, Goodpasture's syndrome, sympathetic opthalamia, phacogenic uveitis, autoimmune haemolytic anaemia, idiopathic thrombocytopenic purpura, idiopathic leucopenia, primary biliary cirrhosis, active chronic hepatitis, cryptogenic cirrhosis, ulcerative colitis, Sjogren's syndrome, and mixed connective tissue diease;

treatment and/or prevention of a disease state caused by the formation and/or complications of atherosclerotic lesions;

diseases which require for therapy:

inhibition of a class II MHC-restricted immune response;

inhibition of an asthmatic response;

inhibition of an allergic response;

inhibition of immune response against transplanted organ or tissue; and

inhibition of elastase activity in atheroma.

The present methods contemplate the use of one or more compounds of Formula I, alone or in combination with other therapeutic agents.

For acute therapy, parenteral administration of a compound of Formula I is preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg, in a manner to maintain the concentration of drug in the plasma at a concentration effective to inhibit cathepsin S. The compounds are administered one to four times daily at a level to achieve a total daily dose of about 0.4 to about 400 mg/kg/day. The precise amount of an inventive compound which is therapeutically effective, and the route by which such compound is best administered, is readily determined by one of ordinary skill in the art by comparing the blood level of the agent to the concentration required to have a therapeutic effect.

The compounds of Formula I may also be administered orally to the patient, in a manner such that the concentration of drug is sufficient to inhibit bone resorption or to achieve any other therapeutic indication as disclosed herein. Typically, a pharmaceutical composition containing the compound is administered at an oral dose of between about 0.1 to about 50 mg/kg in a manner consistent with the condition of the patient. Preferably the oral dose would be about 0.5 to about 20 mg/kg.

No unacceptable toxicological effects are expected when compounds of Formula I are administered in accordance with the present methods.

### Biological Assays

The compounds used in the present methods may be tested in one of several biological assays to determine the concentration of compound which is required to have a given pharmacological effect.

### Determination of Cathepsin S Proteolytic Catalytic Activity

All assays for cathepsin S were carried out with human recombinant enzyme. Standard assay conditions for the determination of kinetic constants used a fluorogenic peptide substrate, typically Cbz-Val-Val-Arg-AMC, and were determined in 100 mM Na acetate at pH 5.5 containing 20 mM cysteine and 5 mM EDTA. Stock substrate solutions were prepared at concentrations of 10 or 20 mM in DMSO with 20 uM final substrate concentration in the assays. All assays contained 10% DMSO. All assays were conducted at ambient temperature. Product fluorescence (excitation at 360 nM; emission at 460 nM) was monitored with a Perceptive Biosystems Cytofluor II fluorescent plate reader. Product progress curves were generated over 20 to 30 minutes following formation of AMC product.

### Inhibition Studies

Potential inhibitors were evaluated using the progress curve method. Assays were carried out in the presence of variable concentrations of test compound. Reactions were initiated by addition of enzyme to buffered solutions of inhibitor and substrate. Data analysis was conducted according to one of two procedures depending on the appearance of the progress curves in the presence of inhibitors. For those compounds whose progress curves were linear, apparent inhibition constants (K_{i,app}) were calculated according to equation 1 (Brandt et al., Biochemitsry, 1989, 28, 140):

v=VₘA/[Kₐ(1+I/K_{i,app})+A] (1)

where v is the velocity of the reaction with maximal velocity Vₘ, A is the concentration of substrate with Michaelis constant of Kₐ, and I is the concentration of inhibitor.

For those compounds whose progress curves showed downward curvature characteristic of time-dependent inhibition, the data from individual sets was analyzed to give k_{obs} according to equation 2:

[AMC]=vₛₛt+(v₀−vₛₛ)[1−exp(−k_{obs}t)]/k_{obs} (2)

where [AMC] is the concentration of product formed over time t, v₀ is the initial reaction velocity and vₛₛ is the final steady state rate. Values for k_{obs} were then analyzed as a linear function of inhibitor concentration to generate an apparent second order rate constant (k_{obs}/inhibitor concentration or k_{obs}/[I]) describing the time-dependent inhibition. A complete discussion of this kinetic treatment has been fully described (Morrison et al., Adv. Enzymol. Relat. Areas Mol. Biol. 1988, 61, 201).

### General

Nuclear magnetic resonance spectra were recorded at either 250 or 400 MHz using, respectively, a Bruker AM 250 or Bruker AC 400 spectrometer. CDCl₃ is deuteriochloroform, DMSO-d₆ is hexadeuteriodimethylsulfoxide, and CD₃OD is tetradeuteriomethanol. Chemical shifts are reported in parts per million (d) downfield from the internal standard tetramethylsilane. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. Continuous wave infrared (IR) spectra were recorded on a Perkin-Elmer 683 infrared spectrometer, and Fourier transform infrared (FTIR) spectra were recorded on a Nicolet Impact 400 D infrared spectrometer. IR and FTIR spectra were recorded in transmission mode, and band positions are reported in inverse wavenumbers (cm⁻¹). Mass spectra were taken on either VG 70 FE, PE Syx API III, or VG ZAB HF instruments, using fast atom bombardment (FAB) or electrospray (ES) ionization techniques. Elemental analyses were obtained using a Perkin-Elmer 240C elemental analyzer. Melting points were taken on a Thomas-Hoover melting point apparatus and are uncorrected. All temperatures are reported in degrees Celsius.

Analtech Silica Gel GF and E. Merck Silica Gel 60 F-254 thin layer plates were used for thin layer chromatography. Both flash and gravity chromatography were carried out on E. Merck Kieselgel 60 (230-400 mesh) silica gel.

Where indicated, certain of the materials were purchased from the Aldrich Chemical Co., Milwaukee, Wis., Chemical Dynamics Corp., South Plainfield, N.J., and Advanced Chemtech, Louisville, Ky.

### EXAMPLES

In the following synthetic examples, temperature is in degrees Centigrade (°C.). Unless otherwise indicated, all of the starting materials were obtained from commercial sources. Without further elaboration, it is believed that one skilled in the art can, using the preceding description, utilize the present invention to its fullest extent. These Examples are given to illustrate the invention, not to limit its scope. Reference is made to the claims for what is reserved to the inventors hereunder.

### Example 1

### Preparation of Benzofuran-2-carboxylic acid {(S)-2-cyclohexyl-1-[3-oxo-1-(pyridine-2-sulfonyl)-azepan-4-ylcarbamoyl]-ethyl}-amide

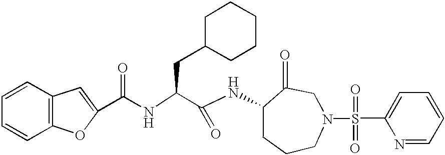

### a.) Allyl-pent-4-enyl-carbamic acid benzyl ester

To a suspension of NaH (1.83 g, 76.33 mmol of 90% NaH) in DMF was added benzyl allyl-carbamic acid benzyl ester (7.3 g, 38.2 mmol) in a dropwise fashion. The mixture was stirred at room temperature for approximately 10 minutes whereupon 5-bromo-1-pentene (6.78 mL, 57.24 mmol) was added in a dropwise fashion. The reaction was heated to 40° C. for approximately 4 hours whereupon the reaction was partitioned between dichloromethane and water. The organic layer was washed with water (2×'s), brine, dried (MgSO₄), filtered and concentrated. Column chromatography of the residue (10% ethyl acetate:hexanes) provided 10.3 grams of the title compound as an oil: MS(EI) 260 (M+H⁺).

### b.) 2,3,4,7-Tetrahydro-azepine-1-carboxylic acid benzyl ester

To a solution of compound of Example 1a (50 g) in dichloromethane was added bis(tricyclohexylphosphine)benzylidine ruthenium (IV) dichloride (5.0 g). The reaction was heated to reflux until complete as determined by TLC analysis. The reaction was concentrated in vacuo. Column chromatography of the residue (50% dichloromethane:hexanes) gave 35 g of the title compound: MS(EI) 232 (M+H⁺).

### c.) 8-Oxa-3-aza-bicyclo[5.1.0]octane-3-carboxylic acid benzyl ester

To a solution of the compound of Example 1b (35 g, 1.5 mol) in CH₂Cl₂ was added m-CPBA (78 g, 0.45 mol). The mixture was stirred overnight at room temperature whereupon it was filtered to remove the solids. The filtrate was washed with water and saturated NaHCO₃ (several times). The organic layer was dried (MgSO₄), filtered and concentrated to give 35 g of the title compound which was of sufficient purity to use in the next step: MS(EI) 248 (M+H⁺), 270 (M+Na⁺).

### d.) 4-azido-3-hydroxy-azepane-1-carboxylic acid benzyl ester

To a solution of the epoxide from Example 1c (2.0 g, 8.1 mmol) in methanol:water (8:1 solution) was added NH₄Cl (1.29 g, 24.3 mmol) and sodium azide (1.58 g, 24.30 mmol). The reaction was heated to 40° C. until complete consumption of the starting epoxide was observed by TLC analysis. The majority of the solvent was removed in vacuo and the remaining solution was partitioned between ethyl acetate and pH 4 buffer. The organic layer was washed with sat. NaHCO₃, water, brine dried (MgSO₄), filtered and concentrated. Column chromatography (20% ethyl acetate:hexanes) of the residue provided 1.3 g of the title compound: MS(EI) 291 (M+H⁺) plus 0.14 g of trans-4-hydroxy-3-azido-hexahydro-1H-azepine

### e.) 4-amino-3-hydroxy-azepane-1-carboxylic acid benzyl ester

To a solution of the azido alcohol of Example 1d (1.1 g, 3.79 mmol) in methanol was added triethylamine (1.5 mL, 11.37 mmol) and 1,3-propanedithiol (1.1 mL, 11.37 mL). The reaction was stirred until complete consumption of the starting material was observed by TLC analysis whereupon the reaction was concentrated in vacuo. Column chromatography of the residue (20% methanol:dichloromethane) provided 0.72 g of the title compound: MS(EI) 265 (M+H⁺).

### f.) 4-tert-Butoxycarbonylamino-3-hydroxy-azepane-1-carboxylic acid benzyl ester

To a stirring solution of 4-amino-3-hydroxy-azepane-1-carboxylic acid benzyl ester (Example 1e, 1.04 g, 3.92 mmol) in THF was added di-tert-butyldicarbonate (0.864 g). After stirring at room temperature for 30 minutes, the reaction mixture was diluted with diethylether and extracted with saturated NaHCO₃. The organic layer was dried over anhydrous Na₂SO₄, filtered, concentrated, and purified by silica gel column to give the title compound as a yellow oil (0.963 g, 2.64 mmol, 67%). MS (ESI): 365.03 (M+H⁺).

### g.) 3-Hydroxy-azepan-4-yl-carbamic acid-tert-butyl ester

To a solution of 4-tert-butoxycarbonylamino-3-hydroxy-azepane-1-carboxylic acid benzyl ester (Example 1f, 0.963 g, 2.64 mmol) in ethyl acetate (16 mL) was added 10% palladium on carbon (500 mg). After stirring the solution at room temperature for 48 hours, the mixture was filtered through celite. The filterate was concentrated to yield the title compound (0.529 g, 2.29 mmol, 87%). MS(ESI): 231.92 (M+H⁺).

### h.) 3-Hydroxy-1-(pyridine-2-sulfonyl)-azepan-4-yl-carbamic acid-tert-butyl ester

To a solution of 3-hydroxy-azepan-4-yl-carbamic acid-tert-butyl ester (Example 1g, 0.529, 2.29 mmol) in DCM (20 mL) was added triethylamine (232 mg) and pyridine-2-sulfonyl chloride (410 mg, 2.32 mmol). After stirring at room temperature for 30 minutes, the mixture was washed with saturated NaHCO₃. The organic layer was dried, filtered, concentrated and purified on a silica gel column to give the title compound as a solid (0.583 g, 1.57 mmol, 68%). MS(ESI): 372.95 (M+H⁺).

### i.) 4-Amino-1-(pyridine-2-sulfonyl)-azepan-3-ol

To a stirring solution of 3-hydroxy-1-(pyridine-2-sulfonyl)-azepan-4-yl-carbamic acid-tert-butyl ester (Example 1h, 0.583 g, 1.57 mmol) in ethyl acetate (0.5 mL) was added HCl (4M in dioxane) (3.9 mL). After stirring the reaction mixture for 30 minutes at room temperature, the mixture was concentrated to yield a white solid. The solid was treated with NaOH and then extracted with ethylacetate. The organic layer was dried, filtered, and concentrated to yield a yellow solid (0.347 g, 1.28 mmol, 81%). MS (ESI) 272.93 (M+H⁺).

### j.) {(S)-2-Cyclohexyl-1-[3-hydroxy-1-(pyridine-2-sulfonyl)-azepan-4-ylcarbamoyl]-ethyl}-carbamic acid-tert-butyl ester

To a solution of 4-amino-1-(pyridine-2-sulfonyl)-azepan-3-ol (Example 1i, 19 mg, 0.070 mmol) in CH₂Cl₂ was added N-Boc-cyclohexylalanine (28.5 mg, 0.106 mmol), 1-hydroxybenzotriazole (16.1 mg, 0.12 mmol), and P-EDC (140 mg, 0.14 mmol)in CH₂Cl₂. After shaking at room temperature overnight, the mixture was treated with PS-Trisamine. After shaking for another 2 hours, the mixture was filtered and concentrated to yield the title compound as a solid. MS (ESI) 525 (M+H⁺).

### k.) (S)-2-Amino-3-cyclohexyl-N-[3-hydroxy-1-(pyridine-2-sulfonyl)-azepan-4-yl]-propionamide

To a stirring solution of {(S)-2-cyclohexyl-1-[3-hydroxy-1-(pyridine-2-sulfonyl)-azepan-4-ylcarbamoyl]-ethyl}-carbamic acid-tert-butyl ester (Example 1j, 34 mg, 0.07 mmol) in CH₂Cl₂ (0.50 mL) was added HCl (4M in dioxane) (0.165 mL). After stirring at room temperature for 30 minutes, the mixture was concentrated, giving a white solid. The white solid was azeotroped with toluene then treated with MP-carbonate (0.35 mmol) in methanol. After four hours of shaking, the mixture was filtered and concentrated to give the title compound as a solid. MS(ESI) 425.03 (M+H⁺).

### l.) Benzofuran-2-carboxylic acid {(S)-2-cyclohexyl-1-[3-hydroxy-1-(pyridine-2-sulfonyl)-azepan-4-ylcarbamoyl]-ethyl}-amide

To a solution of (S)-2-amino-3-cyclohexyl-N-[3-hydroxy-1-(pyridine-2-sulfonyl)-azepan-4-yl]-propionamide (Example 1k, 27 mg, 0.070 mmol) in CH₂Cl₂ was added benzofuran-2-carboxylic acid (17.0 mg, 0.106 mmol), 1-hydroxybenzotriazole (16.1 mg, 0.12 mmol), and P-EDC (140 mg, 0.14 mmol) in CH₂Cl₂. After shaking at room temperature overnight, the mixture was treated with PS-Trisamine. After shaking for another 2 hours, the mixture was filtered and concentrated to yield the title compound as a solid. MS (ESI) 568.79 (M+H)⁺.

### m.) Benzofuran-2-carboxylic acid {(S)-2-cyclohexyl-1-[3-oxo-1-(pyridine-2-sulfonyl)-azepan-4-ylcarbamoyl]-ethyl}-amide

To a stirring solution of benzofuran-2-carboxylic acid {(S)-2-cyclohexyl-1-[3-hydroxy-1-(pyridine-2-sulfonyl)-azepan-4-ylcarbamoyl]-ethyl}-amide (Example 1l, 37 mg, 0.070 mmol) in CH₂Cl₂ (0.5 mL) was added Dess-Martin reagent (45 mg, 0.105 mmol). After stirring for 30 minutes, solutions of sodium thiosulfate (10% in water, 0.50 mL) and saturated aqueous sodium bicarbonate (0.50 mL) were added simultaneously to the reaction. The mixture was then extracted with dichloromethane (2 times). The organic layer was dried, filtered, and concentrated. The residue was purified on a preparative R,R-Whelk-O column by HPLC to yield the two diastereomers of the title compound as solids (first eluting: 4.5 mg, second eluting: 4.5 mg). MS (ESI) 566.87 (M+H⁺); ¹H NMR (400 Hz, CDCl₃): δ8.67(m), 7.95(m), 7.63(m), 7.50(m), 7.02(m), 6.83(m), 5.25(m), 4.76(m), 4.14(t), 3.88(d), 2.74(m), 2.16(m), 1.88(m), 1.66-0.94(m).

### Example 2

### Preparation of 5-(3-Trifluoromethyl-phenyl)-furan-2-carboxylic acid {(S)-2-cyclohexyl-1-[(R)-3-oxo-1-(pyridine-2-sulfonyl)-azepan-4-ylcarbamoyl]-ethyl}-amide

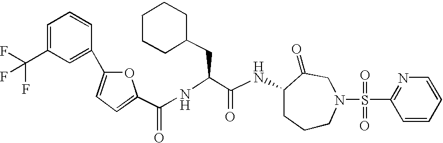

Following the procedure of Example 1(l)-1(m) except substituting 5-(3-trifluoromethylphenyl)-furan-2-carboxylic acid for benzofuran-2-carboxylic acid in step 1(l), the title compound was purified to yield two diastereomers as solids: ¹H-NMR (400 Hz, CDCl₃): δ8.67(m), 7.93(m), 7.58(m), 7.24(m), 6.83(m), 5.18(m), 4.76(m), 4.27(t), 3.85(d), 2.78(m), 2.16(m), 1.85(m), 1.52-1.02(m).

### Example 3

### Preparation of 5-(4-Chloro-phenyl)-furan-2-carboxylic acid {(S)-2-cyclohexyl-1-[3-oxo-1-(pyridine-2-sulfonyl)-azepan-4-ylcarbamoyl]-ethyl}-amide

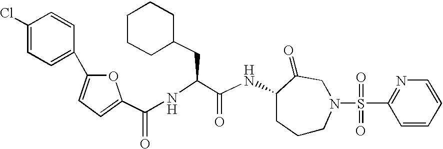

Following the procedure of Example 1(l)-1(m), except substituting 5-(4-chloro-phenyl)-furan-2-carboxylic acid for 2-benzofurancarboxylic acid in step 1(l), the title compound was purified to yield two diastereomers as solids: ¹H-NMR (400 Hz, CDCl₃): δ8.62(m), 7.93(m), 7.65(d), 7.47(m), 7.38(t), 7.20(m), 6.92(m), 6.72(d), 5.18(m), 4.77(m), 4.09(t), 3.84(d), 2.73(m), 2.33-1.02(m).

### Example 4

### Preparation of 5-(4-Chloro-phenyl)-furan-2-carboxlic acid {(S)-2-cyclohexyl-1-[3-oxo-1-(1-oxy-pyridine-2-sulfonyl)-azepan-4-ylcarbamoyl]-ethyl}-amide

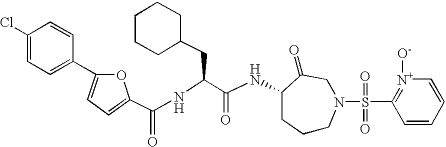

Following the procedure of Example 1(h)-1(m), except substituting 5-(4-chloro-phenyl)-furan-2-carboxylic acid for benzofuran-2-carboxylic acid in step 1(l) and 2-pyridine-N-oxide sulfonyl chloride for pyridine-2-sulfonyl chloride in step 1(h), the title compound was purified to yield two diastereomers as solids: ¹H-NMR (400 Hz, CDCl₃): δ8.26(m), 8.12(t), 7.73-7.21(m), 6.76(t), 5.09(m), 4.82(m), 4.10(d), 3.88(dd), 3.54(s), 2.79(m), 2.19-1.02(m).

### Example 5

### Preparation of 5-(3-Trifluoromethyl-phenyl)-furan-2-carboxylic acid {(S)-2-cyclohexyl-1-[3-oxo-1-(1-oxy-pyridine-2-sulfonyl)-azepan-4-ylcarbamoyl]-ethyl}-amide

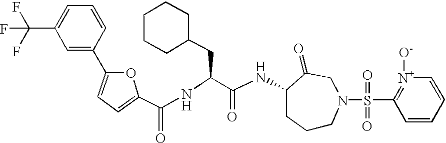

Following the procedure of Example 1(h)-1(m), except 5-(3-trifluoromethyl-phenyl)-furan-2-carboxylic acid for 2-benzofurancarboxylic acid in step 1(1) and 2-pyridine-N-oxide sulfonyl chloride for pyridine-2-sulfonyl chloride in step 1(h), the title compound was purified to yield two diastereomers as solids: ¹H-NMR (400 Hz, CDCl₃): δ8.26(m), 8.11(t), 8.02-7.23(m), 6.86(t), 5.11(m), 4.82(m), 4.14(t), 3.90-3.85(d), 3.16(s), 3.88(m), 2.25-1.02(m).

### Example 6

### Preparation of 5,6-Dimethoxy-benzofuran-2-carboxylic acid {(S)-2-cyclohexyl-1-3-oxo-1-(1-oxy-pyridine-2-sulfonyl)-azepan-4-ylcarbamoyll-ethyl}-amide

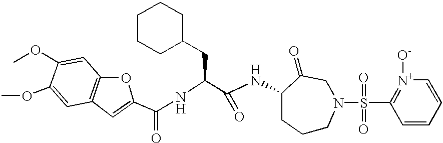

Following the procedure of Example 1(h)-1(m), except 5,6-dimethoxy-benzofuran-2-carboxylic acid in step 1(1) and 2-pyridine-N-oxide sulfonyl chloride for pyridine-2-sulfonyl chloride in step 1(h), the title compound was purified to yield two diastereomers as solids: ¹H-NMR (400 Hz, CDCl₃): δ8.25-7.37(m), 7.07(d), 5.02(m), 4.88(m), 4.12(d), 3.96(s), 3.94(s), 3.84(d), 3.73(s), 2.86(t), 2.20(m), 1.94-1.02(m).

### Example 7

### Preparation of Benzofuran-2-carboxylic acid {(S)-3-methyl-1-[(2,2′,4-trideuterio)-3-oxo-1-(pyridine-2-sulfonyl)-azepan-4-ylcarbamoyl]-butyl}amide

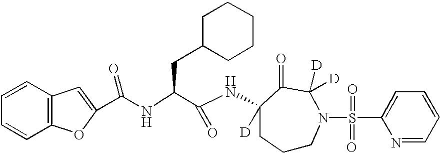

### a.) 4-((S)-2-tert-Butoxycarbonylamino-4-methyl-pentanoylamino)-3-hydroxy-azepan-1-carboxylic acid benzyl ester

To a solution of 4-amino-3-hydroxy-azepane-1-carboxylic acid benzyl ester of Example 1e (720 mg, 2.72 mmol) in CH₂Cl₂ was added EDC (521 mg), HOBt (368 mg) and N-Boc-leucine (630 mg). The reaction was maintained at room temperature until complete consumption of the starting material was observed by TLC analysis. The reaction was diluted with ethyl acetate and washed with 1N HCl, sat. K₂CO₃, water, brine, dried (MgSO₄), filtered and concentrated. Column chromatography of the residue (3% methanol:dichloromethane) gave 1.0 g of the title compound: MS(EI) 478 (M+H⁺).

### b.) [(S)-1-(3-Hydroxy-azepan-4-ylcarbamoyl)-3-methyl-butyl]-carbamic acid tert butyl ester

To a solution of the compound of Example 7a (1.0 g) and 10% Pd/C (catalytic) in ethyl acetate:methanol (2:1 solution) was affixed a balloon of hydrogen. The reaction was stirred until complete consumption of the starting material was observed by TLC analysis. The reaction was filtered to remove the catalyst and the filtrate was concentrated in vacuo to provide 0.82 g of the title compound: MS(EI) 344 (M+H⁺).

### c.) {(S)-1-[3-Hydroxy-1-(pyridine-2-sulfonyl)-azepan-4-ylcarbamoyl]-3-methyl-butyl}-carbamic acid tert-butyl ester

Generation of 2-pyridinesulfonylchloride: A solution of 2-mercaptopyridine (2.23 g in 33 ml 9N HCl) was cooled to 0° C. Chlorine gas was bubbled into the solution for 90 min, taking care to maintain the temperature at 0° C. Ice cooled ethyl acetate was added followed by slow addition of ice cooled sat'd NaHCO₃ until the pH of the water layer was approximately 9. The organic layer were then washed with brine and dried over MgSO₄. Evaporation of the ethyl acetate gave 3.5 g of the crude 2-pyridinesulfonylchloride as a light yellow liquid.

To a solution of [(S)-1-(3-hydroxy-azepan-4-ylcarbamoyl)-3-methyl-butyl]-carbamic acid tert butyl ester of Example 7b (12 g, 34.93 mmol) in dichloromethane was added triethylamine (5.8 mL, 41.92 mmol) followed by the dropwise addition of 2-pyridinesulfonylchloride (7.45 g, 41.92 mmol). The reaction was stirred until complete as determined by TLC analysis. The mixture was then washed with sat. NaHCO₃, water, brine, dried (Na₂SO₄), filtered and concentrated. Column chromatography (75% ethyl acetate:hexanes to 100% ethyl acetate) of the residue provided 15 g of the title compound: MS 484 (M⁺)

### d.) (S)-2-Amino-4-methyl-pentanoic acid-[3-hydroxy-1-(pyridine-2-sulfonyl)-azepan-4-yl]-amide

To a solution of {(S)-1-[3-hydroxy-1-(pyridine-2-sulfonyl)-azepan-4-ylcarbamoyl]-3-methyl-butyl}-carbamic acid tert-butyl ester of Example 7c (14.3 g) in methanol was added 4 M HCl in dioxane. The reaction was stirred at room temperature until complete as determined by TLC analysis whereupon it was concentrated to provide 14 g of the title compound: MS (EI) 385 (M+H⁺).

### e.) Benzofuran-2-carboxylic acid {(S)-3-methyl-1-[3-hydroxy-1-(pyridine-2-sulfonyl)-azepan-4-ylcarbamoyl]-butyl}amide

To a solution of (S)-2-amino-4-methyl-pentanoic acid [3-hydroxy-1-(pyridine-2-sulfonyl)-azepan-4-yl]-amide of Example 7d (0.15 g) in dichloromethane was added TEA (0.11 mL), HOBt (49 mg), EDC (69 mg) and benzofuran-2-carboxylic acid (58 mg). The reaction was stirred until complete. Workup and column chromatography (5% methanol:ethyl acetate) provided the title compound: MS(EI) 529 (M+H⁺).

### f.) Benzofuran-2-carboxylic acid {(S)-3-methyl-1-[3-oxo-1-(pyridine-2-sulfonyl)-azepan-4-ylcarbamoyl]-butyl}amide

To a solution of the alcohol of Example 7e (0.11 g) in DMSO was added TEA (0.17 mL) and pyridine sulfur trioxide complex (99 mg). The reaction was stirred at room temperature for approximately 2 hours whereupon it was partitioned between ethyl acetate and water. The organic layer was washed with brine, dried, filtered and concentrated. Column chromatography of the residue (10% CH₃OH:EtOAc) provided 75 mg of the title compound as a mixture of diastereomers: ¹H NMR (CDCl₃): δ1.0 ( m, 6H), 1.5-2.1 (m, 5H), 2.2 (m, 2H), 2.7 (m, 1H), 3.7 (dd, 1H). 4.0 (m, 1H), 4.7 (m, 2H), 5.0 (m, 1H), 7.2-7.3 (m, 3H), 7.4 (m, 4H), 7.6 (m, 1H), 8.0 (m, 2H), 8.7 (m, 1H); MS(EI): 527 (M+H⁺, 40%).

### g.) of Benzofuran-2-carboxylic acid {(S)-3-methyl-1-[(2,2.,4-trideuterio)-3-oxo-1-(pyridine-2-sulfonyl)-azepan-4-ylcarbamoyl]-butyl}amide

To a solution of benzofuran-2-carboxylic acid {(S)-3-methyl-1-[3-oxo-1-(pyridine-2-sulfonyl)-azepan-4-ylcarbamoyl]-butyl}amide of Example 7f (0.03 g) in D₂O:CD₃OD (0.4:4 mL) was added triethylamine (0.04 mL). The reaction was heated to reflux for 2 hours whereupon it was concentrated and dried under vacuum. The residue was the redissolved in the same mixture and heated to reflux overnight. The reaction was concentrated and the residue purified by column chromatography (5% methanol:dichloromethane) to provide the title compound (0.02 g): ¹HNMR: δ1.0 (m, 6H), 1.5-2.2 (m, 6H), 2.7 (m, 1H), 4.1 (m, 1H), 4.7 (m, 2H), 7.4-8.0 (m, 8H), 8.7 (m, 1H); MS(EI): 529 (M⁺, 45%).

The diastereomeric mixture was separated by HPLC to provide the faster eluting diastereoemer: MS(EI): 530 (M+H⁺,100%) and the slower eluting diastereomer: MS(EI): 530 (M+H⁺,100%).

### Example 8

### Furan-2-carboxylic acid {(S)-2-cyclohexyl-1-[(4S,7R)-7-methyl-3-oxo-1-(pyridine-2-sulfonyl)-azepan-4-ylcarbamoyl]-ethyl}-amide

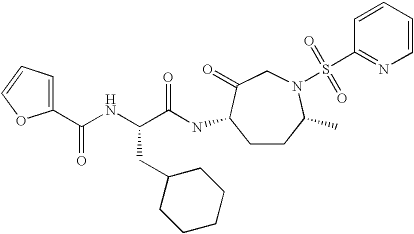

### a. ((R)-2-Iodo-1-methyl-ethyl)-carbamic acid benzyl ester

Triphenylphospine (24 g, 91.8 mmol) was added to a solution of imidazole (12.5 g, 184 mmol) in CH₂Cl₂ (231 ml), then was cooled to 0 degrees C. Iodine (23.3 g, 91.8 mmol) was added to the suspension. The reaction mixture turned yellow, then faintly brown. After 5 minutes ((R)-2-hydroxy-1-methyl-ethyl)-carbamic acid benzyl ester (9.59 g, 45.9 mmol) was added and the reaction mixture was warmed to RT then stirred for 3 h. Then, H₂O (7 ml) was added and the reaction mixture was partitioned between CH₂Cl₂ (300 ml) and H₂O (600 ml). The aqueous layer was extracted again with CH₂Cl₂ (200 ml). The combined organic layer was then washed with a solution of 1:9 aq. saturated Na₂S₂O₃: H₂O (140 ml), then brine (400 ml). The combined organics were dried with MgSO₄, filtered, concentrated in vacuo, then filtered through a plug of silica gel washing with 15% EtOAc/hexanes (1.5 liter). The solution was concentrated in vacuo, then the solid was washed with hexane and the resultant white solid was used in the next reaction without further purification (11 g, 75%).

### b. ((R)-1-Methyl-pent-4-enyl)-carbamic acid benzyl ester

Copper (I) bromide-dimethyl sulfide (1.93 g, 9.4 mmol) was dissolved in distilled THF (24 ml), then was cooled to −78 degrees C. A solution of allyl magnesium chloride (9.4 ml, 2M in THF, Aldrich) was added dropwise, then the solution was stirred for 30 minutes. ((R)-2-Iodo-1-methyl-ethyl)-carbamic acid benzyl ester (1.5 g, 4.7 mmol) in distilled THF (3 ml) was added dropwise, then the reaction was warmed to −40 degrees C. and was stirred for 2.5 h. The reaction mixture was quenched with aq. sat. NH₄Cl (4 ml) at −40 degrees C., warmed to RT and the gray reaction mixture turned sky blue. THF was removed in vacuo. Then, Et₂O was added and the reaction mixture was filtered to remove precipitated solids. The solids were washed with additional Et₂O. The combined organics were extracted with 10% NH₄OH (3×), then brine. The combined organics were dried with MgSO₄, filtered, concentrated in vacuo, then filtered through a plug of silica gel washing with 20% EtOAc/hexanes (100 ml). The solution was concentrated in vacuo, then the resultant colorless oil was used in the next reaction without further purification (0.8 g, 73%).

### c. Allyl-((R)-1-methyl-pent-4-enyl)-carbamic acid benzyl ester

((R)-1-Methyl-pent-4-enyl)-carbamic acid benzyl ester (790 mg, 3.39 mmol) was dissolved in DMF (8 ml) and was cooled to 0 degrees C. Sodium hydride (60% dispersion, 271 mg, 6.78 mmol) was added and the reaction was stirred for 15 minutes. Allyl bromide (1.23 g, 0.88 ml, 10.17 mmol) was added and the reaction mixture was stirred for 3 h at 0 degrees C. H₂O (10 ml) was added, then 2N HCl was added dropwise adjusting the pH to 1. The reaction mixture was extracted with Et₂O (2×50 ml). The combined organics were washed with aq. 2N HCl, then aq. NaHCO₃, then brine. The combined organics were dried with MgSO₄, filtered, concentrated in vacuo, then chromatographed on silica gel (5% EtOAc/hexanes) to yield the title compound as a colorless oil (883 mg, 95%).

### d. 2-Methyl-2,3,4,7-tetrahydro-azepine-1,-carboxylic acid benzyl ester

Allyl-(1-methyl-pent-4-enyl)-carbamic acid benzyl ester (0.872 g, 3.19 mmol) was dissolved in CH₂Cl₂ (10 ml) and a stream of argon gas was bubbled into the reaction mixture for 10 minutes. Then bis(tricyclohexylphosphine)benzylidine ruthenium(IV) dichloride (Strem Chemicals, Gnibbs' catalyst, 19 mg, 0.0227 mmol) was added and the reaction mixture was refluxed for 2 h. Additional bis(tricyclohexylphosphine)benzylidine ruthenium(IV) dichloride (mg, 0.0108 mmol) was added and the reaction mixture was refluxed for an additional 1.5 hours. The reaction was cooled to RT under argon overnight, then was concentrated in vacuo by rotary evaporation, then was chromatographed (silica gel, 5% EtOAc/hexanes) to give the title compound (0.72 g, 92%): 1H NMR: 7.35-7.20 (m, 5H), 5.65 (1H, m), 5.13 (2H, AB), 4.45-4.05 (m, 2H), 3.56 (1H, d), 2.25-2.10 (m, 2H), 1.90-1.60 (m, 2H), 1.12 (3H, d); Liquid Chromatgraphy/Electrospray mass spec: M+H⁺=246.2.

### e. (1S,4R,7R)-4-Methyl-8-oxa-3-aza-bicyclo[5.1.0]octane-3-carboxylic acid benzyl ester

m-Chloro-perbenzoic acid (1.10 g, 57-86% pure) was added to a solution of 2-methyl-2,3,4,7-tetrahydro-azepine-1-carboxylic acid benzyl ester (0.72 g, 2.94 mmol) in CH₂Cl₂ at 0 degrees C. The reaction mixture was stirred for half an hour, then was warmed to RT. Additional m-chloro-perbenzoic acid (0.660 g, 57-86% pure) was added and the reaction was stirred 2 h. The reaction mixture was concentrated in vacuo by rotary evaporation, then 80 ml of 9:1 hexanes/EtOAc was added and the reaction mixture was filtered. The filtrate was concentrated in vacuo by rotary evaporation, then was chromatographed (silica gel, 20% EtOAc:hexanes) to give (1S,4R,7S)-4-methyl-8-oxa-3-aza-bicyclo[5.1.0]octane-3-carboxylic acid benzyl ester (0.450 g, 75%) and the title compound (0.15 g, 25% yield): 1H NMR: 7.42-7.22 (m, 5H), 5.13 (2H, s), 4.50-4.15 (m, 2H), 3.27 (1H, d), 3.12-2.95 (1H, m), 2.15-1.70 (m, 2H), 1.47 (m, 2H), 1.12 (3H, d); Liquid Chromatgraphy/Electrospray mass spec: M+H⁺=262.0.

### f. (2R,5S,6S)-5-Azido-6-hydroxy-2-methyl-azepane-1-carboxylic acid benzyl ester

Sodium azide (0.139 g, 2.14 mmol) was added to a solution of (1S,4R,7R)-4-methyl-8-oxa-3-aza-bicyclo[5.1.0]octane-3-carboxylic acid benzyl ester (0.186 g, 0.71 mmol) and ammonium chloride (0.114 g, 2.14 mmol) in MeOH (1.5 ml) and H₂O (0.15 ml), then was refluxed for 6 h. The reaction mixture was concentrated in vacuo by rotary evaporation, then was diluted with water (5 ml) and extracted with EtOAc (10 ml). The organic layer was then extracted with water, brine, dried with MgSO₄, filtered, concentrated in vacuo by rotary evaporation, and chromatographed (silica gel, 20% EtOAc/hexanes) to yield the title compound (0.192 g, 89%): 7.39-7.30 (m, 5H), 5.15 (2H, s), 4.10-3.67 (m, 2H), 3.10 (1H, d), 1.85-1.53 (m, 4H), 1.09 (3H, d); Liquid Chromatgraphy/Electrospray mass spec: M+H⁺=305.2.

### g. (2R,5S,6S)-5-Amino-6-hydroxy-2-methyl-azepane-1-carboxylic acid benzyl ester

Triphenylphosphine (0.25 g, 0.952 mmol) was added to a solution of (2R,5S,6S)-5-azido-6-hydroxy-2-methyl-azepane-1-carboxylic acid benzyl ester (0.193 g, 0.635 mmol) in THF (10 ml) and H₂O (0.04 ml), then was heated to 45 degrees C. overnight. The reaction mixture was then diluted with toluene (100 ml×2) and was azeotroped in vacuo by rotary evaporation twice. The resulting oil was dissolved in MeOH and HCl in Et₂O and the resulting salt was collected following filtration and was used in the next reaction without further purification (0.27 g, 90%).

### h. (2R,5S, 6S)-5-((S)-2-tert-Butoxycarbonylamino-3-cyclohexyl-propanoylamino)-2-methyl-3-hydroxy-azepane-1-carboxylic acid benzyl ester

1-(3-Dimethylaminopropyl)-3-ethylcarbodiimide (1.0 g, 5.36 mmol) was added to a solution of Boc-cyclohexylalanine (1.2 g, 4.45 mmol), 4-methylmorpoline (1.35 g, 1.50 ml, 13.4 mmol), hydroxybenztriazole (0.72 g, 5.36 mmol), and (2R,5S,6S)-5-Amino-6-hydroxy-2-methyl-azepane-1-carboxylic acid benzyl ester (1.4 g, 4.45 mmol) in DMF (20 ml). The reaction was stirred overnight at RT, then was diluted with EtOAc (100 ml), washed with H₂O (50 ml), brine (50 ml), dried with magnesium sulfate, filtered, concentrated in vacuo by rotary evaporation, and chromatographed (silica gel, 50% EtOAc/hexanes) to yield the title compound (1.70 g, 72%): Electrospray mass spec: M+H⁺=532.4

### i. [(S)-2-Cyclohexyl-1-((3S,4S,7R)-7-methyl-3-hydroxy-azepan-4-ylcarbamoyl)-ethyl]-carbamic acid tert-butyl ester

(2R,5S, 6S)-5-((S)-2-tert-Butoxycarbonylamino-3-cyclohexyl-propanoylamino)-2-methyl-6-hydroxy-azepane-1-carboxylic acid benzyl ester (1.70 g, 3.20 mmol) was dissovled in ethanol(30 ml). Then 10% Pd/C (0.34 g, 0.32 mmol) was added and the reaction was stirred overnight under a balloon filled with hydrogen gas. The reaction mixture was filtered through Celite, concentrated in vacuo by rotary evaporation and was used in the next reaction without further purification (1.2 g): Electrospray mass spec: M+H⁺=398.4.

### j. {(S)-2-Cyclohexyl-1-[(3S,4S,7R)-7-methyl-3-hydroxy-1-(pyridine-2-sulfonyl)-azepan-4-ylcarbamoyl]-ethyl}-carbamic acid tert-butyl ester

2-Pyridine sulfonyl chloride (0.53 g, 3.30 mmol) was added to a solution [(S)-2-Cyclohexyl-1-((3S,4S,7R)-7-methyl-3-hydroxy-azepan-4-ylcarbamoyl)-ethyl]-carbamic acid tert-butyl ester (1.2 g, 3.00 mmol), triethylamine (1.02 g, 10.0 mmol) in CH₂Cl₂ (20 ml) and was stirred at RT for 30 minutes. The reaction mixture was diluted with EtOAc (100 ml), washed with H₂O, brine, dried with magnesium sulfate, filtered, concentrated in vacuo by rotary evaporation, and chromatographed (silica gel, 1:1 hexane/EtOAc ) to yield the title compound (1.3 g, 80%): Electrospray mass spec: M+H⁺=539.2.

### k. (S)-2-Amino-3-cyclohexyl-N-[(3S,4S,7R)-7-methyl-3-hydroxy-1-(pyridine-2-sulfonyl)-azepan-4-yl]-propionamide

HCl in dioxane (4.0 M, 15.0 ml) was added to a stirred solution of {(S)-2-Cyclohexyl-1-[(3S,4S,7R)-7-methyl-3-hydroxy-1-(pyridine-2-sulfonyl)-azepan-4-ylcarbamoyl]-ethyl}-carbamic acid tert-butyl ester (1.30 g, 2.40 mmol) in MeOH (5.0 ml). The reaction mixture was stirred for 2 h at RT, then was concentrated in vacuo by rotary evaporation and was used in the next reaction without further purification (1.2 g).

### 1. Furan-2-carboxylic acid {(S)-2-cyclohexyl-1-[(3S,4S,7R)-7-methyl-3-hydroxy-1-(pyridine-2-sulfonyl)-azepan-4-ylcarbamoyl]-ethyl}-amide

1-(3-Dimethylaminopropyl)-3-ethylcarbodiimide (0.069 g, 0.36 mmol) was added to a solution of furan-2-carboxylic acid (0.040 g, 0.36 mmol), (S)-2-Amino-3-cyclohexyl-N-[(3S,4S,7R)-7-methyl-3-hydroxy-1-(pyridine-2-sulfonyl)-azepan-4-yl]-propionamide (0.15 g, 0.30 mmol), diisopropylethylamine (0.15 g, 0.20 ml, 1.2 mmol), hydroxybenztriazole (0.049 g, 0.36 mmol) in DMF (2.0 ml) and was stirred at RT overnight. The reaction mixture was then warmed to RT and was stirred overnight. The reaction mixture was diluted with EtOAc (30 ml), washed with H₂O, brine, dried with magnesium sulfate, filtered, concentrated in vacuo by rotary evaporation, and chromatographed (silica gel, 2.5% MeOH/CH₂Cl₂) to yield the title compound (0.15 g, 95%): Electrospray mass spec: M+H⁺=533.2.

### m. Furan-2-carboxylic acid {(S)-2-cyclohexyl-1-[(4S,7R)-7-methyl-3-oxo-1-(pyridine-2-sulfonyl)-azepan-4-ylcarbamoyl]-ethyl}-amide

Dess-Martin periodinane (0.15 g, 0.35 mmol) was added to a solution of Furan-2-carboxylic acid {(S)-2-cyclohexyl-1-[(3S,4S,7R)-7-methyl-3-hydroxy-1-(pyridine-2-sulfonyl)-azepan-4-ylcarbamoyl]-ethyl}-amide (0.15 g, 0.28 mmol) in CH₂Cl₂ (2.0 ml) and was stirred at RT for 1 h. The solution was washed with 10% aq. Na₂S₂O₃, then aq. sat. NaHCO₃, then brine. Purification by column chromatography (3%MeOH/CH₂Cl₂) gave the title compound (0.12 g, 80%): 1H NMR: 8.73(d, 1 H), 7.62(m, 2 H), 7.53(m, 2 H), 7.13(s, 1 H), 6.94(d, 1 H), 6.77(d, 1 H), 6.51(m, 1 H), 5.18(m, 1 H), 4.77(d, 1 H), 4.63(m, 1 H), 4.25(m, 1 H), 3.86(d, 1 H), 2.10(m, 2 H), 1.87-0.93(m, 18 H); Electrospray mass spec: M+H⁺=531.2.

### Example 9

### Preparation of Benzofuran-2-carboxylic acid {(S)-2-cyclohexyl-1-[(4S,7R)-7-methyl-3-oxo-1-(pyridine-2-sulfonyl)-azepan-4-ylcarbamoyl]-ethyl}-amide

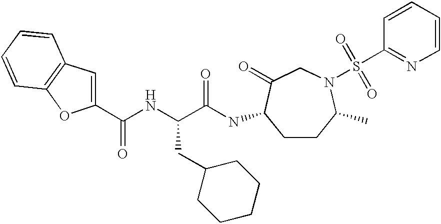

Following the procedure of Example 8 (a-m), except substituting “benzofuran-2-carboxylic acid” for “furan-2-carboxylic acid” gave the title compound: 1H NMR: 8.74(d, 1 H), 7.96(m, 3 H), 7.55(m, 1 H), 7.42(m, 2 H), 7.28(m, 2 H), 6.77(d, 1 H), 6.51(m, 1 H), 5.14(m, 1 H), 4.77(d, 1 H), 4.69(m, 1 H), 4.43(m, 1 H), 3.85(d, 1 H), 2.18(m, 2 H), 1.85-0.98(m, 18 H); Electrospray mass spec: M+H⁺=581.3.

### Example 10

### Preparation of Thiophene-3-carboxylic acid {(S)-2-cyclohexyl-1-[(4S,7R)-7-methyl-3-oxo-1-(pyridine-2-sulfonyl)-azepan-4-ylcarbamoyl]-ethyl}-amide

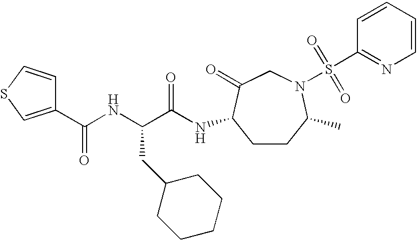

Following the procedure of Example 8 (a-m), except substituting “thiophene-3-carboxylic acid” for “furan-2-carboxylic acid” gave the title compound: 1H NMR: 8.74(d, 1 H), 8.00(m, 2 H), 7.66(d, 1 H), 7.46(m, 3 H), 7.28(d, 1 H), 6.90(d, 1 H), 5.14(m, 1 H), 4.43(m, 1 H), 3.82(d, 1 H), 2.16(m, 2 H), 1.90-0.96(m, 18 H); Electrospray mass spec: M+H⁺=547.2

### Example 11

### Preparation of 3-Methyl-furo[3,2-b]-pyridine-2-carboxylic acid {(S)-2-cyclohexyl-1-[(4S,7R)-7-methyl-3-oxo-1-(pyridine-2-sulfonyl)-azepan-4-ylcarbamoyl]-ethyl}-amide

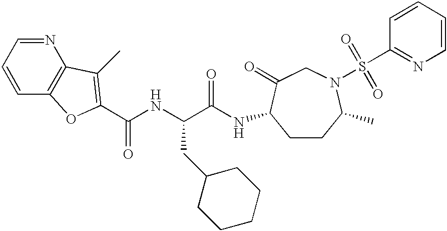

Following the procedure of Example 8 (a-m), except substituting “3-methyl-furo[3,2-b]-pyridine-2-carboxylic acid” for “furan-2-carboxylic acid” gave the title compound: 1H NMR: 8.75(d, 1 H), 7.98(m, 2 H), 7.55(m, 1 H), 7.40(m, 2 H), 7.33(m, 1 H), 6.75(d, 1 H), 6.50(m, 1 H), 5.09(m, 1 H), 4.79(d, 1 H), 4.68(m, 1 H), 4.47(m, 1 H), 3.87(d, 1 H), 2.55 (s,3 H), 2.17(m, 1 H), 1.93-0.93(m, 19 H); Electrospray mass spec: M+H⁺=596.4.

### Example 12

### Preparation of 5-(2-Morpholin-4-yl-ethoxy)-benzofuran-2-carboxylic acid {(S)-2-cyclohexyl-1-[3-oxo-1-(pyridine-2-sulfonyl)-azepan-4-ylcarbamoyl]-ethyl}-amide

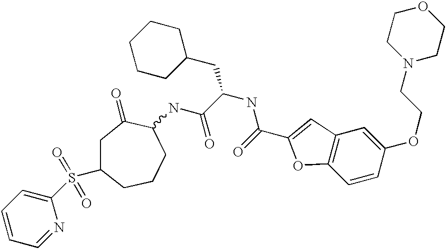

Following the procedure of Example 8 (i-m), except substituting “(3S,4S)-4-Amino-3-hydroxy-azepane-1-carboxylic acid benzyl ester (as described in Marquis, Robert W., et al J. Med. Chem. 44 2001) for “(2R,5S,6S)-5-Amino-6-hydroxy-2-methyl-azepane-1-carboxylic acid benzyl ester” and “5-(2-Morpholin-4-yl-ethoxy)-benzofuran-2-carboxylic acid” for “furan-2-carboxylic acid” gave the title compound: 1H NMR: 8.54(s, 1H), 8.00(m, 2 H), 7.55-7.05(m, 7 H), 5.16(m, 1 H), 4.81-3.52(m, 15 H), 3.14(br, 2 H), 2.71 (t, 1 H), 2.21-0.95(m, 16 H); Electrospray mass spec: M+H⁺=712.4.

### Example 13

### Preparation of 4-Methyl-2-pyridin-2-yl-thiazole-5-carboxylic acid {(S)-2-cyclohexyl-1-[3-oxo-1-(pyridine-2-sulfonyl)-azepan-4-ylcarbamoyl]-ethyl}-amide

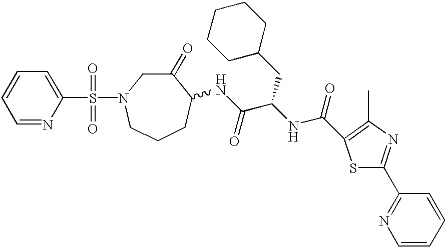

Following the procedure of Example 8 (i-m), except substituting “(3S,4S)-4-Amino-3-hydroxy-azepane-1-carboxylic acid benzyl ester” for “(2R,5S,6S)-5-Amino-6-hydroxy-2-methyl-azepane-1-carboxylic acid benzyl ester” and “4-Methyl-2-pyridin-2-yl-thiazole-5-carboxylic acid” for “furan-2-carboxylic acid” gave the title compound: 1H NMR: 8.66(d, 1 H), 8.55(d, 1 H), 7.98(m, 2 H), 7.65(m, 2 H), 7.50(m, 2 H), 7.44(m, 1 H), 7.31(t, 1 H), 7.06(d, 1 H), 5.17(m, 1 H), 4.79(m, 1 H), 4.65(d, 2 H), 4.00(d, 1 H), 3.83(d, 1 H), 2.75 (t, 1 H), 2.59(s, 3H), 2.40(m, 2 H), 1.84-0.90(m, 15 H);; Electrospray mass spec: M+H⁺=625.4.

### Example 14

### Preparation of 5-Pyridin-2-yl-thiophene-2-carboxylic acid {(S)-2-cyclohexyl-1-[3-oxo-1-(pyridine-2-sulfonyl)-azepan-4-ylcarbamoyl]-ethyl}-amide

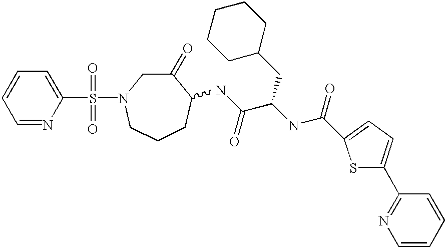

Following the procedure of Example 8 (i-m), except substituting “(3S,4S)-4-Amino-3-hydroxy-azepane-1-carboxylic acid benzyl ester” for “(2R,5S,6S)-5-Amino-6-hydroxy-2-methyl-azepane-1-carboxylic acid benzyl ester” and “5-Pyridin-2-yl-thiophene-2-carboxylic acid” for “furan-2-carboxylic acid” gave the title compound: 1H NMR: 8.68(d, 1H), 8.54(d, 1 H), 7.93(m, 2 H), 7.71(m, 2 H), 7.53(m, 2 H), 7.48(m, 1 H), 7.31(t, 1 H), 7.03(d, 1 H), 5.16(m, 1 H), 4.78(m, 1 H), 4.65(d, 2 H), 4.10(d, 1 H), 3.82(d, 1 H), 2.76(t, 1 H), 2.40(m, 2 H), 1.88-0.89(m, 15 H); Electrospray mass spec: M+H⁺=610.2.

### Example 15

### Preparation of Furan-2-carboxylic acid {(S)-2-cyclohexyl-1-[3-oxo-1-(pyridine-2-sulfonyl)-azepan-4-ylcarbamoyl]-ethyl}-amide

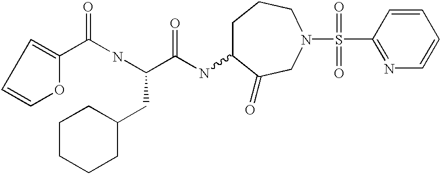

Following the procedure of Example 8 (i-m), except substituting “(3S,4S)-4-Amino-3-hydroxy-azepane-1-carboxylic acid benzyl ester” for “(2R,5S,6S)-5-Amino-6-hydroxy-2-methyl-azepane-1-carboxylic acid benzyl ester” gave the title compound: 1H NMR: 8.70-8.68(d, 1H), 7.98(m, 2H), 7.53(m, 2H), 7.16-7.12(m, 2H), 6.81-6.75(m, 1H), 6.53(s, 1H), 5.31-5.10(m, 1H), 4.81-4.68(m, 2H), 4.13-4.09(d, 1H), 3.93-3.80(d, 1H), 2.77-2.69(m, 1H), 2.26-0.90(m, 17H); Electrospray mass spec: M+H⁺=517.4.

### Example 16

### Preparation of Thiophene-2-carboxylic acid {(S)-2-cyclohexyl-1-[3-oxo-1-(pyridine-2-sulfonyl)-azepan-4-ylcarbamoyl]-ethyl}-amide

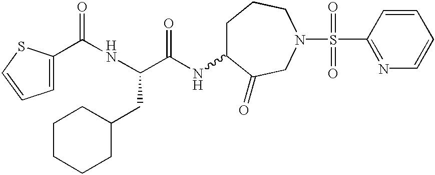

Following the procedure of Example 8 (i-m), except substituting “(3S,4S)-4-Amino-3-hydroxy-azepane-1-carboxylic acid benzyl ester” for “(2R,5S,6S)-5-Amino-6-hydroxy-2-methyl-azepane-1-carboxylic acid benzyl ester” and “Thiophene-2-carboxylic acid” for “furan-2-carboxylic acid” gave the title compound: 1H NMR: 8.70-8.69(d, 1H), 7.99-7.82(m, 2H), 7.60-7.51(m, 3H), 7.12-7.10(m, 2H), 6.55-6.53(d, 1H), 5.14-5.11(m, 1H), 4.78-4.67(m, 2H), 4.10-4.07(d, 1H), 3.89-3.84(d, 1H), 2.81-2.74(m, 1H), 2.26-2.16(m, 2H), 1.86-0.90(m, 15H);; Electrospray mass spec: M+H⁺=533.2.

### Example 17

### Preparation of Thiophene-3-carboxylic acid {(S)-2-cyclohexyl-1-[3-oxo-1-(pyridine-2-sulfonyl)-azepan-4-ylcarbamoyl]-ethyl}-amide

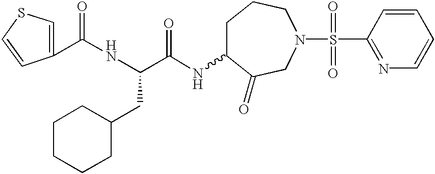

Following the procedure of Example 8 (i-m), except substituting “(3S,4S)-4-Amino-3-hydroxy-azepane-1-carboxylic acid benzyl ester” for “(2R,5S,6S)-5-Amino-6-hydroxy-2-methyl-azepane-1-carboxylic acid benzyl ester” and “Thiophene-3-carboxylic acid” for “furan-2-carboxylic acid” gave the title compound: 1H NMR: 8.72-8.71(d, 1H), 8.15-8.00(m, 3H), 7.56-7.30(m, 3H), 7.15-7.12(br, 1H), 6.70(br, 1H), 5.20(m, 1H), 4.90-4.70(m, 2H), 4.15(m, 1H), 3.90(d, 1H), 2.90-2.70(m, 1H), 2.28-0.97(m, 17H); Electrospray mass spec: M+H³⁰ =533.4.

### Example 18

### Preparation of 5-Methyl-thiophene-2-carboxylic acid {(S)-2-cyclohexyl-1-[3-oxo-1-(pyridine-2-sulfonyl)-azepan-4-ylcarbamoyl]-ethyl}-amide

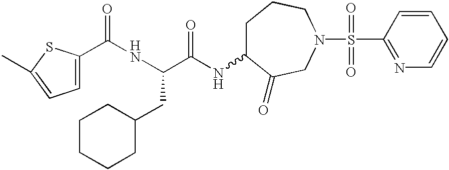

Following the procedure of Example 8 (i-m), except substituting “(3S,4S)-4-Amino-3-hydroxy-azepane-1-carboxylic acid benzyl ester” for “(2R,5S,6S)-5-Amino-6-hydroxy-2-methyl-azepane-1-carboxylic acid benzyl ester” and “5-Methyl-thiophene-2-carboxylic acid” for “furan-2-carboxylic acid” gave the title compound: 1H NMR: 8.69-8.67(d, 1H), 7.97-7.90(m, 2H), 7.52-7.28(m, 3H), 6.74-6.49(m, 2H), 5.18-5.08(m, 1H), 4.77-4.63(m, 2H), 4.28-4.26(d, 1H), 3.87-3.80(d, 1H), 2.78-2.66(m, 1H), 2.51(s, 3H), 2.25-0.88(m, 17H);; Electrospray mass spec: M+H⁺=547.2.

### Example 19

### Preparation of 3-Methyl-thiophene-2-carboxylic acid {(S)-2-cyclohexyl-1-[3-oxo-1-(pyridine-2-sulfonyl)-azepan-4-ylcarbamoyl]-ethyl}-amide

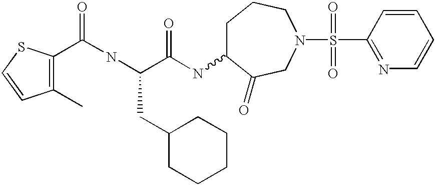

Following the procedure of Example 8 (i-m), except substituting “(3S,4S)-4-Amino-3-hydroxy-azepane-1-carboxylic acid benzyl ester” for “(2R,5S,6S)-5-Amino-6-hydroxy-2-methyl-azepane-1-carboxylic acid benzyl ester” and “3-Methyl-thiophene-2-carboxylic acid” for “furan-2-carboxylic acid” gave the title compound: 1H NMR: 8.69-8.68(d, 1H), 7.97-7.89(m, 2H), 7.53-7.50(m, 1H), 7.32-7.17(m, 2H), 6.91-6.84(d, 1H), 6.34-6.32(d, 1H), 5.16-5.11(m, 1H), 4.79-4.70(m, 2H), 4.31-4.10(d, 1H), 3.85-3.81(d, 1H), 2.76-2.69(m, 1H), 2.55(s, 3H), 2.26-0.89(m, 17H); Electrospray mass spec: M+H⁺=547.2.

### Example 20

### Preparation of 3-Ethoxy-thiophene-2-carboxylic acid {(S)-2-cyclohexyl-1-[3-oxo-1-(pyridine-2-sulfonyl)-azepan-4-ylcarbamoyl]-ethyl}-amide

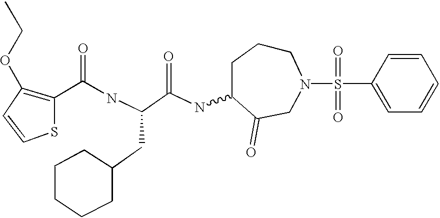

Following the procedure of Example 8 (i-m), except substituting “(3S,4S)-4-Amino-3-hydroxy-azepane-1-carboxylic acid benzyl ester” for “(2R,5S,6S)-5-Amino-6-hydroxy-2-methyl-azepane-1-carboxylic acid benzyl ester” and “3-Ethoxy-thiophene-2-carboxylic acid” for “furan-2-carboxylic acid” gave the title compound: 1H NMR: 8.69-8.67(d 1H), 7.96-7.90(m, 2H), 7.60-7.28(m, 4H), 6.92-6.83(d, 1H), 5.15-5.10(m, 1H), 4.74-4.56(m, 2H), 4.30-4.08(m, 3H), 3.84-3.77(d, 1H), 2.72-2.66(m, 1H), 2.25-0.89(m, 20H); Electrospray mass spec: M+H⁺=577.2.

### Example 21

### Preparation of 4-Bromo-N-{(S)-2-cyclohexyl-1-[3-oxo-1-(pyridine-2-sulfonyl)-azepan-4-ylcarbamoyl]-ethyl}-benzamide

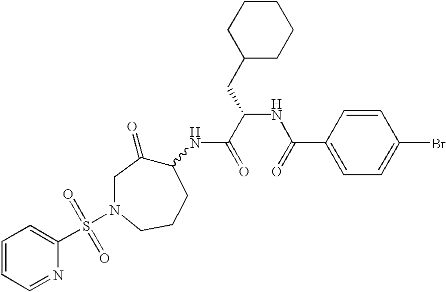

Following the procedure of Example 8 (i-m), except substituting “(3S,4S)-4-Amino-3-hydroxy-azepane-1-carboxylic acid benzyl ester” for “(2R,5S,6S)-5-Amino-6-hydroxy-2-methyl-azepane-1-carboxylic acid benzyl ester” and “4-bromo-benzoic acid” for “furan-2-carboxylic acid” gave the title compound: 1H NMR: 8.71(d, 1 H), 8.00(m, 2 H), 7.69(d, 2 H), 7.52(m, 3 H), 7.26(d, 1 H), 6.91(d, 1 H), 5.22(m, 1 H), 4.77(m, 2 H), 4.14(d, 1 H), 3.85(d, 1 H), 2.71(t, 1 H), 2.31(m, 2 H), 1.86-0.91(m, 15 H); Electrospray mass spec: M+H³⁰ =605.2.

### Example 22

### Preparation of Cyclobutanecarboxylic acid {(S)-2-cyclohexyl-1-[3-oxo-1-(pyridine-2-sulfonyl)-azepan-4-ylcarbamoyl]-ethyl}-amide

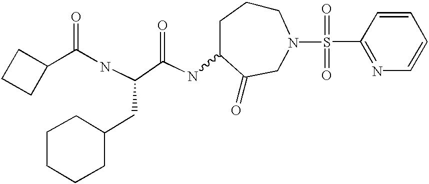

Following the procedure of Example 8 (i-m), except substituting “(3S,4S)-4-Amino-3-hydroxy-azepane-1-carboxylic acid benzyl ester” for “(2R,5S,6S)-5-Amino-6-hydroxy-2-methyl-azepane-1-carboxylic acid benzyl ester” and “Cyclobutanecarboxylic acid” for “furan-2-carboxylic acid” gave the title compound: 1H NMR: 8.68 (d, 1H), 7.97-7.90(m, 2H), 7.71-7.48(m, 1H), 7.19-7.12(d, 1H), 6.81-6.79(d, 1H), 5.08(m, 1H), 4.72-4.48(m, 2H), 4.05-4.01(d, 1H), 3.86-3.79(d, 1H), 3.11-3.05(m,1H), 2.80-2.70(m, 1H), 2.32-0.80(m, 23H); Electrospray mass spec: M+H⁺=505.4.

### Example 23

### Preparation of Cyclopentanecarboxylic acid {(S)-2-cyclohexyl-1-[3-oxo-1-(pyridine-2-sulfonyl)-azepan-4-ylcarbamoyl]-ethyl}-amide

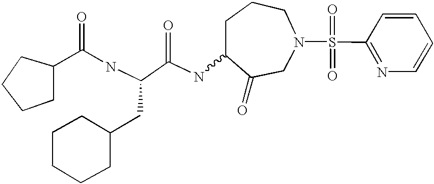

Following the procedure of Example 8 (i-m), except substituting “(3S,4S)-4-Amino-3-hydroxy-azepane-1-carboxylic acid benzyl ester” for “(2R,5S,6S)-5-Amino-6-hydroxy-2-methyl-azepane-1-carboxylic acid benzyl ester” and “Cyclopentanecarboxylic acid” for “furan-2-carboxylic acid” gave the title compound: 1H NMR: 8.70-8.69(d, 1H), 7.99-7.92(m, 2H), 7.55-7.51(m, 1H), 7.09-7.08(d, 1H), 5.89-5.87(d, 1H), 5.10(m, 1H), 4.71-4.70(d, 1H), 4.65(m, 1H), 4.07-4.03(d, 1H), 3.89-3.84(d, 1H), 2.82-2.58(m, 2H), 2.15(m, 2H), 1.90-0.89(m, 23H); Electrospray mass spec: M+H⁺=519.4.

### Example 24

### Preparation of (S)-Tetrahydro-furan-2-carboxylic acid {(S)-2-cyclohexyl-1-[3-oxo-1-(pyridine-2-sulfonyl)-azepan-4-ylcarbamoyl]-ethyl}-amide

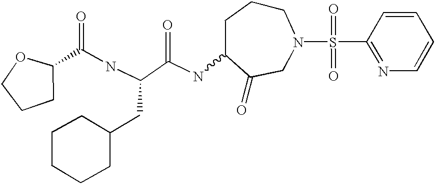

Following the procedure of Example 8 (i-m), except substituting “(3S,4S)-4-Amino-3-hydroxy-azepane-1-carboxylic acid benzyl ester” for “(2R,5S,6S)-5-Amino-6-hydroxy-2-methyl-azepane-1-carboxylic acid benzyl ester” and “(S)-Tetrahydro-furan-2-carboxylic acid” for “furan-2-carboxylic acid” gave the title compound: 1H NMR: 8.67(d, 1H), 7.96(m, 2H), 7.53(m, 1H), 6.96(m, 2H), 5.13(m, 1H), 4.75(m, 1H), 4.41(m, 2H), 4.07-3.91(m, 4H), 2.68(m, 1H), 2.35-0.92 (m, 21H); Electrospray mass spec: M+H⁺=521.4.

### Example 25

### Preparation of (R)-Tetrahydro-furan-2-carboxylic acid {(S)-2-cyclohexyl-1-[3-oxo-1-(pyridine-2-sulfonyl)-azepan-4-ylcarbamoyl]-ethyl}-amide

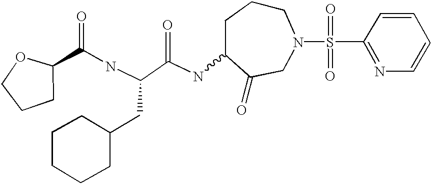

Following the procedure of Example 8 (i-m), except substituting “(3S,4S)-4-Amino-3-hydroxy-azepane-1-carboxylic acid benzyl ester” for “(2R,5S,6S)-5-Amino-6-hydroxy-2-methyl-azepane-1-carboxylic acid benzyl ester” and “(R)-Tetrahydro-furan-2-carboxylic acid” for “furan-2-carboxylic acid” gave the title compound: 1H NMR: 8.71(d, 1H), 7.96(m, 2H), 7.53(m, 1H), 7.12(m, 2H), 5.10(m, 1H), 4.72(m, 1H), 4.46(m, 2H), 4.11-3.95(m, 4H), 2.74(m, 1H), 2.35-0.92 (m, 21H); Electrospray mass spec: M+H⁺=521.4.

### Example 26

### Preparation of Furan-3-carboxylic acid {(S)-2-cyclohexyl-1-[3-oxo-1-(pyridine-2-sulfonyl)-azepan-4-ylcarbamoyl]-ethyl}-amide

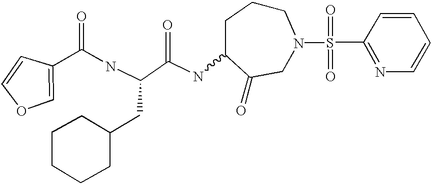

Following the procedure of Example 8 (i-m), except substituting “(3S,4S)-4-Amino-3-hydroxy-azepane-1-carboxylic acid benzyl ester” for “(2R,5S,6S)-5-Amino-6-hydroxy-2-methyl-azepane-1-carboxylic acid benzyl ester” and “furan-3-carboxylic acid” for “furan-2-carboxylic acid” gave the title compound: 1H NMR: 8.70-8.68(d, 1H), 7.99-7.92(m, 3H), 7.54-7.44(m, 2H), 7.19-7.18(d, 1H), 6.59-6.57(m, 2H), 5.14-5.09(m, 1H), 4.79-4.63(m, 2H), 4.07-4.04(d, 1H), 3.89-3.84(d, 1H), 2.83-2.76(m, 1H), 2.23-0.91(m, 17H); Electrospray mass spec: M+H⁺=517.4.

### Example 27

### Preparation of 5-Pyridin-2-yl-thiophene-2-carboxylic acid {(S)-2-cyclohexyl-1-[3-oxo-1-(1-oxy-pyridine-2-sulfonyl)-azepan-4-ylcarbamoyl]-ethyl}-amide

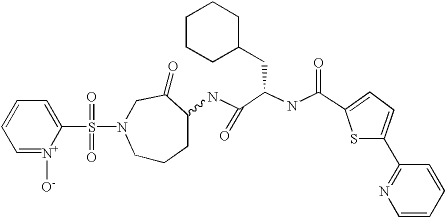

Following the procedure of Example 8 (i-m), except substituting “(3S,4S)-4-Amino-3-hydroxy-azepane-1-carboxylic acid benzyl ester” for “(2R,5S,6S)-5-Amino-6-hydroxy-2-methyl-azepane-1-carboxylic acid benzyl ester” and “5-Pyridin-2-yl-thiophene-2-carboxylic acid” for “furan-2-carboxylic acid” and “oxy-pyridine-2-sulfonyl chloride” for “2-pyridine sulfonyl chloride” gave the title compound: 1H NMR: 8.55(d, 1 H), 8.05(d, 1 H), 8.03(d, 1 H), 7.73-7.09(m, 9 H), 5.06(m, 1 H), 4.80(m, 2 H), 4.11(d, 1 H), 3.84(d, 1 H), 2.90(t, 1 H), 2.22(m, 1 H), 2.10-0.88(m, 15 H); Electrospray mass spec: M+H⁺=626.4.

### Example 28

### Preparation of 4-Methyl-2-pyridin-2-yl-thiazole-5-carboxylic acid {(S)-2-cyclohexyl-1-[3-oxo-1-(1-oxy-pyridine-2-sulfonyl)-azepan-4-ylcarbanioyl]-ethyl}-amide

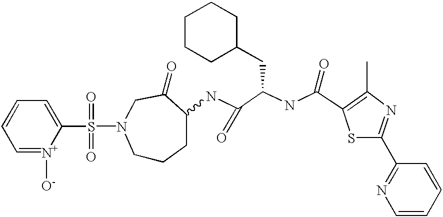

Following the procedure of Example 8 (i-m), except substituting, “(3S,4S)-4-Amino-3-hydroxy-azepane-1-carboxylic acid benzyl ester” for “(2R,5S,6S)-5-Amino-6-hydroxy-2-methyl-azepane-1-carboxylic acid benzyl ester” and “4-Methyl-2-pyridin-2-yl-thiazole-5-carboxylic acid” for “furan-2-carboxylic acid” and “oxy-pyridine-2-sulfonyl chloride” for “2-pyridine sulfonyl chloride” gave the title compound: 1H NMR: 8.53(d, 1 H), 8.08(d, 1 H), 8.03(d, 1 H), 7.77-7.05(m, 9 H), 5.03(m, 1 H), 4.75(m, 2 H), 4.13(d, 1 H), 3.80(d, 1 H), 2.88(t, 1 H), 2.67(s, 3 H), 2.22 (m, 1 H), 2.10-0.88(m, 15 H); Electrospray mass spec: M+H⁺=641.4.

### Example 29

### Preparation of 5-(2-Morpholin-4-yl-ethoxy)-benzofuran-2-carboxylic acid {(S)-2-cyclohexyl-1-[3-oxo-1-(1-oxy-pyridine-2-sulfonyl)-azepan-4-ylcarbamoyl]-ethyl}-amide

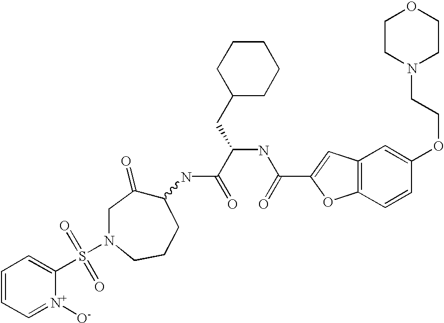

Following the procedure of Example 8 (i-m), except substituting “(3S,4S)-4-Amino-3-hydroxy-azepane-1-carboxylic acid benzyl ester” for “(2R,5S,6S)-5-Amino-6-hydroxy-2-methyl-azepane-1-carboxylic acid benzyl ester” and “5-(2-Morpholin-4-yl-ethoxy)-benzofuran-2-carboxylic acid” for “furan-2-carboxylic acid” and “oxy-pyridine-2-sulfonyl chloride” for “2-pyridine sulfonyl chloride” gave the title compound: 1H NMR: 8.23(br, 1 H), 8.06(d, 2 H), 7.48-7.00(m, 8 H), 5.03(m, 1 H), 4.80(m, 2 H), 4.59(m, 2 H), 4.27(m, 2 H), 4.09-3.33(m, 9 H), 3.29(m, 2 H), 2.80(m, 2 H), 2.27-0.88(m, 14 H); Electrospray mass spec: M+H⁺=712.4.

### Example 30

### Preparation of Furan-2-carboxylic acid {(S)-2-cyclohexyl-1-[3-oxo-1-(1-oxy-pyridine-2-sulfonyl)-azepan-4-ylcarbamoyl]-ethyl}-amide

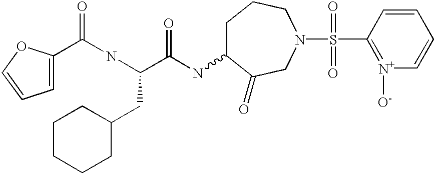

Following the procedure of Example 8 (i-m), except substituting “(3S,4S)-4-Amino-3-hydroxy-azepane-1-carboxylic acid benzyl ester” for “(2R,5S,6S)-5-Amino-6-hydroxy-2-methyl-azepane-1-carboxylic acid benzyl ester” and “Furan-2-carboxylic acid” for “furan-2-carboxylic acid” and “oxy-pyridine-2-sulfonyl chloride” for “2-pyridine sulfonyl chloride” gave the title compound: 1H NMR: 8.24-8.23(d, 1H), 8.14-8.11(m, 1H), 7.50-7.39(m, 3H), 7.14(d, 1H), 7.01-6.99(d, 1H), 6.78-6.76(d, 1H), 6.52-6.51(d, 1H), 5.04-4,91(m, 2H), 4.72-4.66(d, 1H), 4.14-4.10(d, 1H), 3.93-3.88(d, 1H), 2.85-2.79(m, 1H), 2.25-0.94(m, 17H); Electrospray mass spec: M+H⁺=533.4.

### Example 31

### Preparation of Furan-3-carboxylic acid {(S)-2-cyclohexyl-1-[3-oxo-1-(1-oxy-pyridine-2-sulfonyl)-azepan-4-ylcarbamoyl]-ethyl}-amide

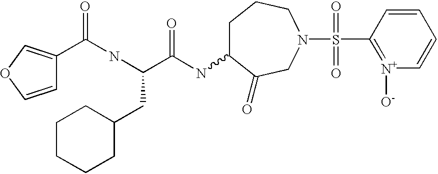

Following the procedure of Example 8 (i-m), except substituting “(3S,4S)-4-Amino-3-hydroxy-azepane-1-carboxylic acid benzyl ester” for “(2R,5S,6S)-5-Amino-6-hydroxy-2-methyl-azepane-1-carboxylic acid benzyl ester” and “Furan-3-carboxylic acid” for “furan-2-carboxylic acid” and “oxy-pyridine-2-sulfonyl chloride” for “2-pyridine sulfonyl chloride” gave the title compound: 1H NMR: 8.25-8.23(d, 1H), 8.14-8.11(m, 1H), 7.97(s, 1H), 7.51-7.39(m, 3H), 7.04-7.03(d, 1H), 6.67(s, 1H), 6.50-6.48(d, 1H), 5.06-4.88 (m, 2H), 4.74-4.68(m, 1H), 4.13-4.09(d, 1H), 3.93-3.88(d, 1H), 2.86-2.79(m, 1H), 2.23-0.93(m, 17H); Electrospray mass spec: M+H⁺=533.4.

### Example 32

### Preparation of Thiophene-3-carboxylic acid {(S)-2-cyclohexyl-1-[3-oxo-1-(1-oxy-pyridine-2-sulfonyl)-azepan-4-ylcarbamoyl]-ethyl}-amide

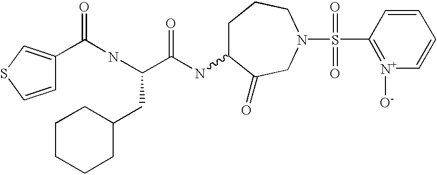

Following the procedure of Example 8 (i-m), except substituting “(3S,4S)-4-Amino-3-hydroxy-azepane-1-carboxylic acid benzyl ester” for “(2R,5S,6S)-5-Amino-6-hydroxy-2-methyl-azepane-1-carboxylic acid benzyl ester” and “Thiophene-3-carboxylic acid” for “furan-2-carboxylic acid” and “oxy-pyridine-2-sulfonyl chloride” for “2-pyridine sulfonyl chloride” gave the title compound: 1H NMR: 8.24-8.22(d, 1H), 8.12-8.09(m, 1H), 7.95(s, 1H), 7.49-7.19(m, 5H), 6.59-6.57(d, 1H), 5.05-5.01 (m, 1H), 4.83-4.74(m, 2H), 4.10-4.06(d, 1H), 3.92-3.87(d, 1H), 2.91-2.85(m, 1H), 2.26-0.92(m, 17H); Electrospray mass spec: M+H⁺=549.4.

### Example 33

### Preparation of Thiophene-2-carboxylic acid {(S)-2-cyclohexyl-1-[3-oxo-1-(1-oxy-pyridine-2-sulfonyl)-azepan-4-ylcarbamoyl]-ethyl}-amide

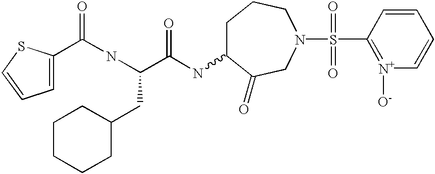

Following the procedure of Example 8 (i-m), except substituting “(3S,4S)-4-Amino-3-hydroxy-azepane-1-carboxylic acid benzyl ester” for “(2R,5S,6S)-5-Amino-6-hydroxy-2-methyl-azepane-1-carboxylic acid benzyl ester” and “Thiophene-2-carboxylic acid” for “furan-2-carboxylic acid” and “oxy-pyridine-2-sulfonyl chloride” for “2-pyridine sulfonyl chloride” gave the title compound: 1H NMR: 8.24-8.23(d, 1H), 8.13-8.10(m, 1H), 7.58-7.38(m, 4H), 7.11-7.07(m, 2H), 6.79-6.77(d, 1H), 5.04-4.69(m, 3H), 4.12-4.08(d, 1H), 3.92-3.87(d, 1H), 2.85-2.79(m, 1H), 2.21-0.90(m, 17H); Electrospray mass spec: M+H⁺=549.4.

### Example 34

### Preparation of 5-Methyl-thiophene-2-carboxylic acid {(S)-2-cyclohexyl-1-[3-oxo-1-(1-oxy-pyridine-2-sulfonyl)-azepan-4-ylcarbamoyl]-ethyl}-amide

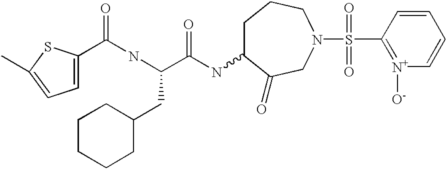

Following the procedure of Example 8 (i-m), except substituting “(3S,4S)-4-Amino-3-hydroxy-azepane-1-carboxylic acid benzyl ester” for “(2R,5S,6S)-5-Amino-6-hydroxy-2-methyl-azepane-1-carboxylic acid benzyl ester” and “5-Methyl-thiophene-2-carboxylic acid” for “furan-2-carboxylic acid” and “oxy-pyridine-2-sulfonyl chloride” for “2-pyridine sulfonyl chloride” gave the title compound: 1H NMR: 8.23-8.22(d, 1H), 8.11-8.08(d, 1H), 7.49-7.24(m, 4H), 6.75-6.74(s, 1H), 6.62-6.60(d, 1H), 5.03-4.71 (m, 3H), 4.09-4.05(d, 1H), 3.90-3.85(d, 1H), 2.88-2.83(m, 1H), 2.67(s, 3H), 2.35-0.88(m, 17H); Electrospray mass spec: M+H⁺=563.2.

### Example 35

### Preparation of 3-Methyl-thiophene-2-carboxylic acid {(S)-2-cyclohexyl-1-[3-oxo-1-(1-oxy-pyridine-2-sulfonyl)-azepan-4-ylcarbamoyl]-ethyl }-amide

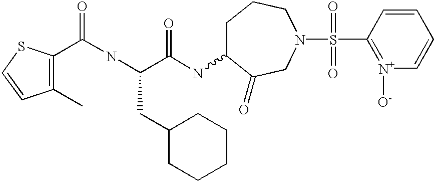

Following the procedure of Example 8 (i-m), except substituting “(3S,4S)-4-Amino-3-hydroxy-azepane-1-carboxylic acid benzyl ester” for “(2R,5S,6S)-5-Amino-6-hydroxy-2-methyl-azepane-1-carboxylic acid benzyl ester” and “3-Methyl-thiophene-2-carboxylic acid” for “furan-2-carboxylic acid” and “oxy-pyridine-2-sulfonyl chloride” for “2-pyridine sulfonyl chloride” gave the title compound: 1H NMR: 8.23-8.22(d, 1H), 8.11-8.09(d, 1H), 7.49-7.17(m, 4H), 6.93-6.91(s, 1H), 6.27(m, 1H), 5.06-4.70(m, 3H), 4.14-4.11(d, 1H), 3.91-3.86(d, 1H), 2.87-2.81(m, 1H), 2.56(s, 3H), 2.28-0.93 (m, 17H); Electrospray mass spec: M+H⁺=563.2.

### Example 36

### Preparation of 3-Ethoxy-thiophene-2-carboxylic acid {(S)-2-cyclohexyl-1-[3-oxo-1-(1-oxy-pyridine-2-sulfonyl)-azepan-4-ylcarbamoyl]-ethyl}-amide

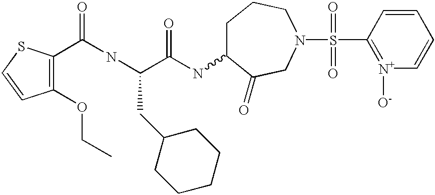

Following the procedure of Example 8 (i-m), except substituting “(3S,4S)-4-Amino-3-hydroxy-azepane-1-carboxylic acid benzyl ester” for “(2R,5S,6S)-5-Amino-6-hydroxy-2-methyl-azepane-1-carboxylic acid benzyl ester” and “3-Ethoxy-thiophene-2-carboxylic acid” for “furan-2-carboxylic acid” and “oxy-pyridine-2-sulfonyl chloride” for “2-pyridine sulfonyl chloride” gave the title compound: 1H NMR: 8.24-8.22(d, 1H), 8.11-8.09(d, 1H), 7.60-7.31(m, 5H), 6.88-6.87 (d, 1H), 5.06-4.65(m, 3H), 4.37-4.27(m, 1H), 4.12-4.08(d, 1H), 3.88-3.83(d, 1H), 2.84-2.77(m, 1H), 2.28-0.92(m, 21H); Electrospray mass spec: M+H⁺=593.2.

### Example 37

### Preparation of Selenophene-2-carboxylic acid {(S)-2-cyclohexyl-1-[(R)-7-methyl-3-oxo-1-(pyridine-2-sulfonyl)-azepan-4-ylcarbamoyl]-ethyl}-amide

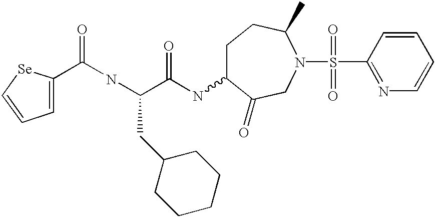

Following the procedure of Example 8 (i-m), except substituting “selenophene-2-carboxylic acid” for “furan-2-carboxylic acid” gave the title compound: 1H NMR: 8.64(d, 1 H), 8.14(d, 1 H), 7.84(m, 2 H), 7.64(d, 1 H), 7.42(m, 1 H), 7.22(m, 1 H), 6.88(d, 1 H), 6.60(d, 1 H), 5.01(m, 1 H), 4.71(d, 1 H), 4.50(m, 1 H), 4.34(m, 1 H), 3.77(d, 1 H), 2.05(m, 2 H), 1.78-0.82(m, 18 H); Electrospray mass spec: M+H⁺=593.2.

### Example 38

### Preparation of Furan-2-carboxylic acid [(S)-2-cyclohexyl-1-((4S,7R)-7-methyl-3-oxo-1-propyl-azepan-4-ylcarbamoyl)-ethyl]-amide

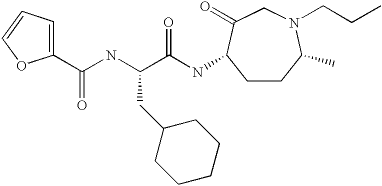

### a. [(S)-2-Cyclohexyl-1-((3S,4S,7R)-3-hydroxy-7-methyl-1-propyl-azepan-4-ylcarbamoyl)-ethyl]-carbamic acid tert-butyl ester

[(S)-2-Cyclohexyl-1-((3S,4S ,7R)-3-hydroxy-7-methyl-azepan-4-ylcarbamoyl)-ethyl]-carbamic acid-tert-butyl ester (Example 1a-I, 1.5 g, 3.78 mmol) was dissolved in CH₂Cl₂(30 mL), then propionaldehyde (0.41 mL, 5.67 mmol) was added. Then, sodium borohydride (1.6 g, 7.56 mmol) was added and the reaction mixture was stirred at RT for 1 h. The reaction mixture was concentrated in vacuo by rotary evaporation, then the filtrate (silica gel, 1-4% MeOH/CH₂Cl₂) to yield the title compound as a white solid (84%, 1.4 g): Electrospray mass spec: M+H⁺=440.4.

### b. (S)-2-Amino-3-cyclohexyl-N-((3S,4S,7R)-3-hydroxy-7-methyl-1-propyl-azepan-4-yl)-propionamide

HCl in dioxane (4.0 M, 15 ml) was added to a stirred solution of [(S)-2-Cyclohexyl-1-((3S,4S,7R)-3-hydroxy-7-methyl-1-propyl-azepan-4-ylcarbamoyl)-ethyl]-carbamic acid tert-butyl ester (1.4 g, 3.0 mmol) in MeOH (5 ml). The reaction mixture was stirred for 2 h at RT, then was concentrated in vacuo by rotary evaporation and was used in the next reaction without further purification (1.4 g).

### c. Furan-2-carboxylic acid [(S)-2-cyclohexyl-1-((3S,4S,7R)-3-hydroxy-7-methyl-1-propyl-azepan-4-ylcarbamoyl)-ethyl]-amide

1-(3-Dimethylaminopropyl)-3-ethylcarbodiimide (0.10 g, 0.53 mmol) was added to a solution of furan-2-carboxylic acid (0.059 g, 0.53 mmol), (S)-2-Amino-3-cyclohexyl-N-((3S,4S,7R)-3-hydroxy-7-methyl-1-propyl-azepan-4-yl)-propionamide (0.15 g, 0.36 mmol), 4-methylmorpholine (0.14 g, 0.16 ml, 1.44 mmol), hydroxybenztriazole (0.071 g, 0.53 mmol) in DMF (2.0 ml) and was stirred at RT overnight. The reaction mixture was then warmed to RT and was stirred overnight. The reaction mixture was diluted with EtOAc (30 ml), washed with H₂O, brine, dried with magnesium sulfate, filtered, concentrated in vacuo by rotary evaporation, and chromatographed (silica gel, 2.5% MeOH/CH₂Cl₂) to yield the title compound (0.12 g, 76%): Electrospray mass spec: M+H⁺=434.2.

### d. Furan-2-carboxylic acid [(S)-2-cyclohexyl-1-((4S,7R)-7-methyl-3-oxo-1-propyl-azepan-4-ylcarbamoyl)-ethyl]-amide

Sulfur trioxide-pyridine complex (0.0.35 g, 2.2 mmol) was added to a solution of Furan-2-carboxylic acid [(S)-2-cyclohexyl-1-((3S,4S,7R)-3-hydroxy-7-methyl-1-propyl-azepan-4-ylcarbamoyl)-ethyl]-amide (0.19 g, 0.44 mmol) in DMSO (4.0 ml) and triethylamine (0.61 ml, 4.4 mmol) was stirred at RT for 1 h. The reaction mixture was diluted with water, then was extracted with EtOAc. Then, the organic layer was was extracted with brine. The combined organics were dried with magnesium sulfate, filtered, concentrated in vacuo, and purified by column chromatography (3% methanol/methylene chloride) gave the title compound (0.15 mg, 79%): 1H NMR: 7.44(s, 1 H), 7.11(d, 1 H), 7.04(d, 1 H), 6.92(d, 1 H), 6.49(d, 1 H), 5.29(m, 1 H), 4.69(m, 1 H), 3.40(d, 1 H), 3.08(m, 2 H), 2.51(m, 2 H), 1.88-0.81(m, 29 H); Electrospray mass spec: M+H⁺=432.2.

### Example 39

### Preparation of Thiophene-3-carboxylic acid [(S)-2-cyclohexyl-1-((4S,7R)-7-methyl-3-oxo-1-propyl-azepan-4-ylcarbamoyl)-ethyl]-amide

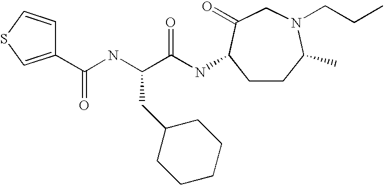

Following the procedure of Example 38 (a-c), except substituting “thiophene-3-carboxylic acid” for “furan-2-carboxylic acid” gave the title compound: 1H NMR: 7.62(d, 1 H), 7.40(d, 1 H), 7.04(d, 1 H), 6.80(d, 1 H), 6.45(d, 1 H), 5.27(m, 1 H), 4.66(m, 1 H), 3.44(d, 1 H), 3.09(m, 2 H), 2.54(m, 2 H), 1.87-0.87(m, 29 H); Electrospray mass spec; M+H⁺=448.4.

### Example 40

### Preparation of Benzofuran-2-carboxylic acid [(S)-2-cyclohexyl-1-((4S,7R)-7-methyl-3-oxo-1-propyl-azepan4-ylcarbamoyl)-ethyl]-amide

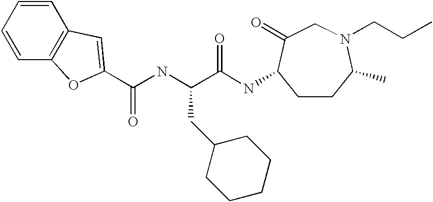

Following the procedure of Example 38 (a-c), except substituting “benzofuran-2-carboxylic acid” for “furan-2-carboxylic acid” gave the title compound: 1H NMR: 7.98(d, 1 H), 7.45(m, 2 H), 7.27(s, 2 H), 6.90(d, 1 H), 6.50(d, 1 H), 5.28(m, 1 H), 4.67(m, 1 H), 3.40(d, 1 H), 3.06(m, 2 H), 2.56(m, 2 H), 1.88-0.80(m, 29 H); Electrospray mass spec: M+H⁺=482.4.

### Example 41

### Preparation of 2,2,4-Trideutero-Furan-2-carboxylic acid {(S)-2-cyclohexyl-1-[(4S,7R)-7-methyl-3-oxo-1-(pyridine-2-sulfonyl)-azepan-4-ylcarbamoyl]-ethyl}-amide

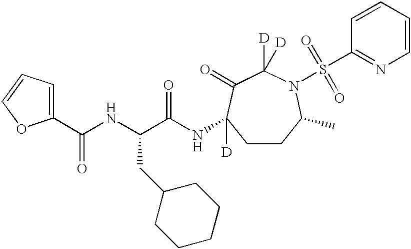

a. Furan-2-carboxylic acid {(S)-2-cyclohexyl-1-[(4S,7R)-7-methyl-3-oxo-1-(pyridine-2-sulfonyl)-azepan-4-ylcarbamoyl]-ethyl}-amide is dissolved in d4-methanol (CD₃OD) and D₂O (10:1), then triethyl amine is added and the reaction mixture is stirred for 3 days. Azeotroping with toluene by concentrating in vacuo provides the title compound.

### Example 42

### Preparation of Thiophene-3-carboxylic acid {(S)-3,3-dimethyl-1-[(4S,7R)-7-methyl-3-oxo-1-(pyridine-2-sulfonyl)-azepan-4-ylcarbamoyl]-butyl}-amide

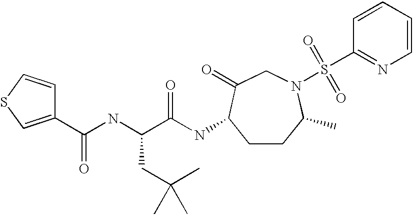

Following the procedure of Example 8 (a-m), except substituting “N-Boc-tert-butylalanine” for “Boc-L-cyclohexylalanine” and “thiophene-3-carboxylic acid” for “furan-2-carboxylic acid” gave the title compound: 1H NMR: 8.72(m, 1 H), 7.96(m, 2 H), 7.48(m, 2 H), 7.00(m, 3 H), 6.60(m, 2 H), 5.18(m, 1 H), 4.67(m, 2 H ), 4.42(m, 1 H), 3.88(m, 1H), 2.87(m, 2 H), 2.22(m, 2 H), 1.95(m, 1 H), 1.70(m, 2 H), 1.01(m, 12 H); Electrospray mass spec: M+H⁺=521.4.

### Example 43

### Preparation of Furan-2-carboxylic acid {(S)-3,3-dimethyl-1-[(4S,7R)-7-methyl-3-oxo-1-(pyridine-2-sulfonyl)-azepan-4-ylcarbamoyl]-butyl}-amide

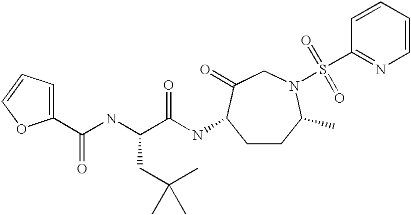

Following the procedure of Example 8 (a-m), except substituting “N-Boc-tert-butylalanine” for “Boc-L-cyclohexylalanine” gave the title compound: 1H NMR: 8.73(d, 1 H), 7.95(m, 3 H), 7.54(m, 1 H), 7.41(m, 1H), 7.32(m, 1H), 7.26(s, 1 H), 7.01(d, 1 H), 6.56(d, 1 H), 5.08(m, 1 H), 4.73(m, 2 H), 4.43(m, 1 H), 3.88(d, 1 H), 2.18(m, 2 H), 1.70(m, 3 H), 1.04(s, 9 H), 0.98(d, 3 H); Electrospray mass spec: M+H⁺=505.4.

### Example 44

### Preparation of Thieno[3,2-b]thiophene-2-carboxylic acid {(S)-3,3-dimethyl-1-[(4S,7R)-7-methyl-3-oxo-1-(pyridine-2-sulfonyl)-azepan-4-ylcarbamoyl]-butyl}-amide

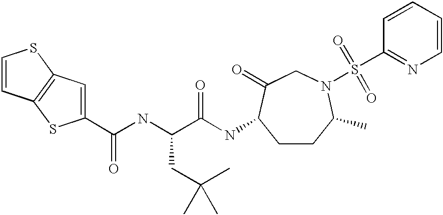

Following the procedure of Example 8 (a-m), except substituting “N-Boc-tert-butylalanine” for “Boc-L-cyclohexylalanine” and “thieno[3,2-b]thiophene-2-carboxylic acid” for “furan-2-carboxylic acid” gave the title compound: 1H NMR: 8.73(d, 1 H), 7.92(m, 3 H), 7.52(m, 2 H), 7.27(m, 1 H), 7.09(br, 1 H), 6.80(br, 1 H), 5.10(m, 1 H), 4.77(m, 2 H), 4.40(m, 1 H), 3.87(d, 1 H), 1.90(m, 5 H), 1.05(s, 9 H), 0.95(d, 3 H); Electrospray mass spec: M+H⁺=577.2.

The above specification and Examples fully disclose how to make and use the compounds of the present invention. However, the present invention is not limited to the particular embodiments described hereinabove, but includes all modifications thereof within the scope of the following claims. The various references to journals, patents and other publications which are cited herein comprise the state of the art and are incorporated herein by reference as though fully set forth.

We claim:

1. A method of inhibiting cathepsin S, comprising administering to a patient in need thereof an effective amount of a compound of Formula I: 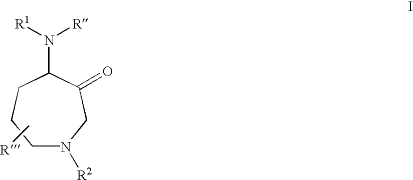
wherein:
R¹ is selected from the group consisting of: 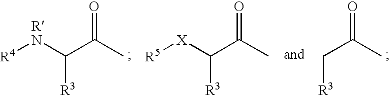
R² is selected from the group consisting of: H, C₁₋₆alkyl, C₃₋₆cycloalkyl-C₀₋₆alkyl, Ar-C₀₋₆alkyl, Het-C₀₋₆alkyl, R⁹C(O)—, R⁹C(S)—, R⁹SO₂—, R⁹OC(O)—, R⁹R¹¹NC(O)—, R⁹R¹¹NC(S)—, 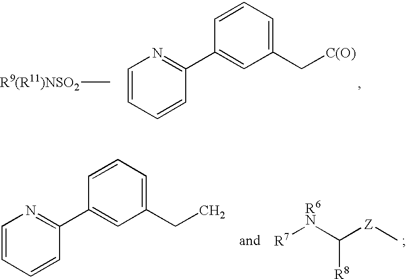
R³ is selected from the group consisting of: H, C₁₋₆alkyl, C₃₋₆cycloalkyl-C₀₋₆alkyl, C₂₋₆alkenyl, C₂₋₆alkynyl, HetC₀₋₆alkyl, ArC₀₋₆alkyl, Ar—ArC₀₋₆alkyl, Ar—HetC₀₋₆alkyl, Het-ArC₀₋₆alkyl, and Het-HetC₀₋₆alkyl;
R³ and R′ may be connected to form a pyrrolidine, piperidine or morpholine ring;
R⁴ is selected from the group consisting of: H, C₁₋₆alkyl, C₃₋₆cycloalkyl-C₀₋₆alkyl, Ar—C₀₋₆alkyl, Het-C₀₋₆alkyl, R⁵C(O)—, R⁵C(S)—, R⁵SO₂—, R⁵OC(O)—, R⁵R¹³NC(O)—, and R⁵R¹³NC(S)—;
R⁵ is selected from the group consisting of: H, C₁₋₆alkyl, C₂₋₆alkenyl, C₂₋₆alkynyl, C₃₋₆cycloalkyl-C₀₋₆alkyl, At—C₀₋₆alkyl and Het-C₀₋₆alkyl;
R⁶ is selected from the group consisting of: H, C₁₋₆alkyl, C₃₋₆cycloalkyl-C₀₋₆alkyl, At—C₀₋₆alkyl, and Het-C₀₋₆alkyl;
R⁷ is selected from the group consisting of: H, C₁₋₆alkyl, C₃₋₆cycloalkyl-C₀₋₆alkyl, At—C₀₋₆alkyl, Het-C₀₋₆alkyl, R¹⁰C(O)—, R¹⁰C(S)—, R¹⁰SO₂—, R¹⁰OC(O)—, R¹⁰R¹⁴NC(O)—, and R¹⁰R¹⁴NC(S)—;
R⁸ is selected from the group consisting of: H, C₁₋₆alkyl, C₂₋₆alkenyl, C₂₋₆alkynyl, HetC₀₋₆alkyl and ArC₀₋₆alkyl;
R⁹ is selected from the group consisting of: C₁₋₆alkyl, C₃₋₆cycloalkyl-C₀₋₆alkyl, At—C₀₋₆alkyl and Het-C₀₋₆alkyl;
R¹⁰ is selected from the group consisting of: C₁₋₆alkyl, C₃₋₆cycloalkyl-C₀₋₆alkyl, At—C₀₋₆alkyl and Het-C₀₋₆alkyl;
R¹¹ is selected from the group consisting of: H, C₁₋₆alkyl, At—C₀₋₆alkyl, and Het-C₀₋₆alkyl;
R¹² is selected from the group consisting of: H, C₁₋₆alkyl, At—C₀₋₆alkyl, and Het-C₀₋₆alkyl;
R¹³ is selected from the group consisting of: H, C₁₋₆alkyl, At—C₋6alkyl, and Het-C₀₋₆alkyl;
R¹⁴ is selected from the group consisting of: H, C₁₋₆alkyl, At—C₀₋₆alkyl, and Het-C₀₋₆alkyl;
R′ is selected from the group consisting of: H, C₁₋₆alkyl, At—C₀₋₆alkyl, and Het-C₀₋₆alkyl;
R″ is selected from the group consisting of: H, C₁₋₆alkyl, At—C₀₋₆alkyl, or Het-C₀₋₆alkyl;
R′″ is selected from the group consisting of: H, C₁₋₆alkyl, C₃₋₆cycloalkyl-C₀₋₆alkyl, At—C₀₋₆alkyl, and Het-C₀₋₆alkyl;
X is selected from the group consisting of: CH₂, S, and O;
Z is selected from the group consisting of: C(O) and CH₂;
and pharmaceutically acceptable salts, hydrates and solvates thereof.

2. A method according to claim 1 wherein in said compound R¹ is

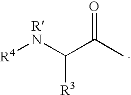

3. A method according to claim 2 wherein in said compound R³ is C₃₋₆cycloalkyl-C₀₋₆alkyl.

4. A method according to claim 3 wherein in said compound R³ is cyclohexylmethyl.

5. A method according to claim 2 wherein in said compound R⁴ is R⁵C(O)—.

6. A method according to claim 5 wherein in said compound R⁵ is selected from the group consisting of: C₁₋₆alkyl, C₃₋₆cycloalkyl-C₀₋₆alkyl, At—C₀₋₆alkyl and Het-C₀₋₆alkyl.

7. A method according to claim 6 wherein in said compound R⁵ is selected from the group consisting of:
furanyl;
benzofuranyl;
thiophenyl;
furo[3,2-b]-pyridine-2-yl;
thiazolyl;
phenyl;
cyclobutyl;
cyclopentyl;
tetrahydrofuranyl;
selenophenyl; and
thieno[3,2-b]thiophenyl.

8. A method according to claim 6 wherein in said compound R⁵ is selected from the group consisting of:
furan-2-yl and furan-3-yl;
benzofuran-2-yl;
thiophene-3-yl and thiophene-2-yl;
furo[3,2-b]-pyridine-2-yl;
thiazole-5-yl;
tetrahydrofuran-2-yl;
selenophene-2-yl; and
thieno[3,2-b]thiophene-2-yl.

9. A method according to claim 6 wherein in said compound R⁵ is selected from the group consisting of:
aryl substituted furanyl;
C₁₋₆alkoxy substituted benzofuranyl;
Het-C₀₋₆alkyl-thiophenyl, C₁₋₆alkyl-thiophenyl and C₁₋₆alkoxy-thiophenyl,
C₁₋₆alkyl-furo[3,2-b]-pyridine-2-yl,
Het-C₀₋₆alkyl-thiazolyl; and
halogen substituted phenyl.

10. A method according to claim 6 wherein in said compound R⁵ is selected from the group consisting of:
5-(3-trifluoromethyl-phenyl)-furan-2-yl and 5-(4-chloro-phenyl)-furan-2-yl;
5,6-dimethoxy-benzofuran-2-yl and 5-(2-morpholin-4-yl-ethoxy)benzofuran-2-yl;
5-pyridin-2-yl-thiophene-2-yl, 5-methyl-thiophene-2-yl, 3-methyl-thiophene-2-yl; and 3-ethoxy-thiophene-2-yl;
3-methyl-furo[3,2-b]-pyridine-2-yl;
4-methyl-2-pyridin-2-yl-thiazole-5-yl; and
4-bromophenyl.

11. A method according to claim 1 wherein in said compound R′ is H.

12. A method according to claim 1 wherein in said compound R″ is H.

13. A method according to claim 1 wherein in said compound R′″ is selected from the group consisting of: H and C₁₋₆alkyl.

14. A method according to claim 1 wherein in said compound R″ is H and R′″ is selected from the group consisting of: H and C₁₋₆alkyl.

15. A method according to claim 13 wherein in said compound R′″ is H.

16. A method according to claim 13 wherein in said compound R′″ is C₁₋₆alkyl.

17. A compound according to claim 16 wherein C₁₋₆alkyl is selected from the group consisting of: 5-, 6- and 7-C₁₋₆alkyl.

18. A compound according to claim 17 wherein 5-, 6- and 7-C₁₋₆alkyl is selected from the group consisting of: 5-, 6- or 7-methyl, -ethyl, -propyl, -butyl, -pentyl, and -hexyl.

19. A compound according to claim 21 wherein 5-, 6- and 7-C₁₋₆alkyl is selected from the group consisting of: 5-, 6- and 7-methyl.

20. A compound according to claim 16 wherein C₁₋₆alkyl is selected from the group consisting of: 6- and 7-C₁₋₆alkyl.

21. A compound according to claim 20 wherein 6- and 7-C₁₋₆alkyl is selected from the group consisting of: 6- or 7-methyl, -ethyl, -propyl, -butyl, -pentyl, and -hexyl.

22. A compound according to claim 21 wherein 6- and 7-C₁₋₆alkyl is selected from the group consisting of: 6- and 7-methyl.

23. A compound according to claim 16 wherein C₁₋₆alkyl is 7-C₁₋₆alkyl.

24. A compound according to claim 23 wherein 7-C₁₋₆alkyl is selected from the group consisting of: 7-methyl, -ethyl, -propyl, -butyl, -pentyl, and -hexyl.

25. A compound according to claim 24 wherein 7-C₁₋₆alkyl is 7-methyl.

26. A compound according to claim 16 of Formula Ia: 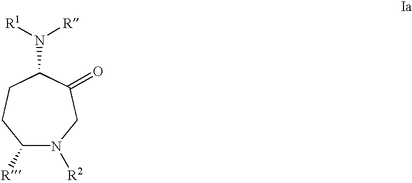
wherein R′″ is cis-7-C₁₋₆alkyl.

27. A compound according to claim 26 wherein R′″ is cis-7-methyl.

28. A method according to claim 1 wherein in said compound R² is R⁹SO₂.

29. A method according to claim 28 wherein in said compound R⁹ is Het-C₀₋₆alkyl.

30. A method according to claim 29 wherein Het-C₀₋₆alkyl is selected from the group consisting of: pyridinyl and 1-oxy-pyridinyl.

31. A method according to claim 30 wherein R⁹ is pyridin-2-yl.

32. A method according to claim 1 wherein in said compound:
R¹ is 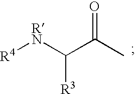
R² is R⁹SO₂;
R³ is C₃₋₆cycloalkyl-C₀₋₆alkyl;
R⁴ is R⁵C(O);
R⁵ is Het-C₀₋₆alkyl;
R⁹ is Het-C₀₋₆alkyl;
R′ is H
R″ is H; and
R′″ is C₁₋₆alkyl.

33. A method according to claim 1 wherein in said compound:
R³ is cyclohexylmethyl;
R⁵ is selected from the group consisting of: furan-2-yl and thiophene-3-yl;
R⁹ is pyridin-2-yl; and
R′″ is 7-methyl.

34. A method according to claim 1 wherein said compound is selected from the group consisting of: 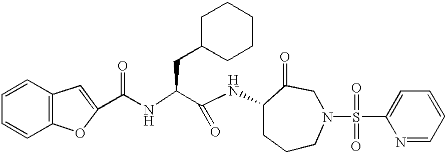
Benzofuran-2-carboxylic acid {(S)-2-cyclohexyl-1-[3-oxo-1-(pyridine-2-sulfonyl)-azepan-4-ylcarbamoyl]-ethyl}-amide; 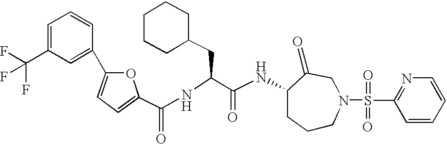
5-(3-Trifluoromethyl-phenyl)-furan-2-carboxylic acid {(S)-2-cyclohexyl-1-[3-oxo-1-(pyridine-2-sulfonyl)-azepan-4-ylcarbamoyl]-ethyl}-amide; 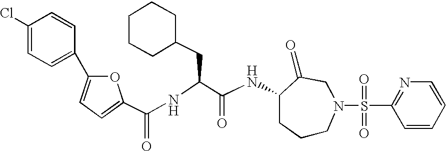
5-(4-Chloro-phenyl)-furan-2-carboxylic acid {(S)-2-cyclohexyl-1-[3-oxo-1-(pyridine-2-sulfonyl)-azepan-4-ylcarbamoyl]-ethyl}-amide; 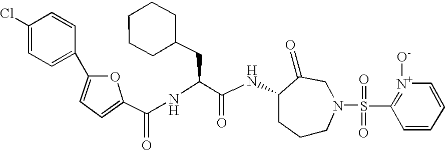
5-(4-Chloro-phenyl)-furan-2-carboxylic acid {(S)-2-cyclohexyl-1-[3-oxo-1-(1-oxy-pyridine-2-sulfonyl)-azepan-4-ylcarbamoyl]-ethyl}-amide; 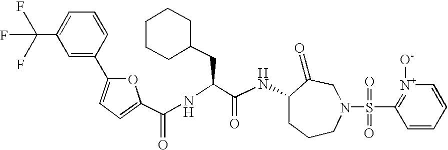
5-(3-Trifluoromethyl-phenyl)-furan-2-carboxylic acid {(S)-2-cyclohexyl-1-[3-oxo-1-(1-oxy-pyridine-2-sulfonyl)-azepan-4-ylcarbamoyl]-ethyl}-amide; 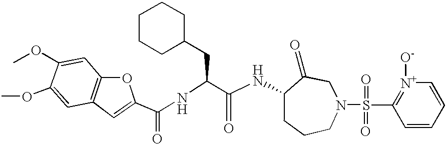
5,6-Dimethoxy-benzofuran-2-carboxylic acid {(S)-2-cyclohexyl-1-[3-oxo-1-(1-oxy-pyridine-2-sulfonyl)-azepan-4-ylcarbamoyl]-ethyl}-amide; and 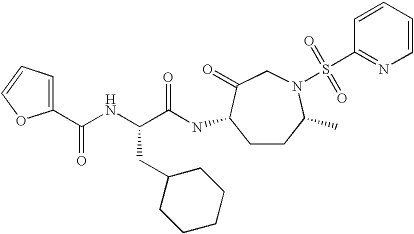
furan-2-carboxylic acid {(S)-2-cyclohexyl-1-[(4S,7R)-7-methyl-3-oxo-1-(pyridine-2-sulfonyl)-azepan-4-ylcarbamoyl]-ethyl}-amide; 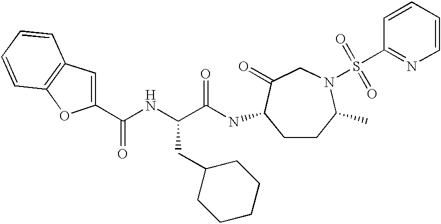
benzofuran-2-carboxylic acid {(S)-2-cyclohexyl-1-[(4S,7R)-7-methyl-3-oxo-1-(pyridine-2-sulfonyl)-azepan-4-ylcarbamoyl]-ethyl}-amide; 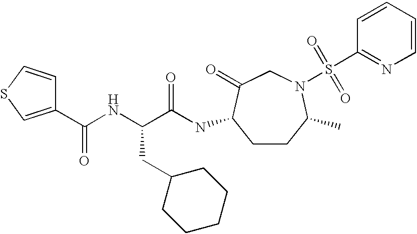
thiophene-3-carboxylic acid {(S)-2-cyclohexyl-1-[(4S,7R)-7-methyl-3-oxo-1-(pyridine-2-sulfonyl)-azepan-4-ylcarbamoyl]-ethyl}-amide; 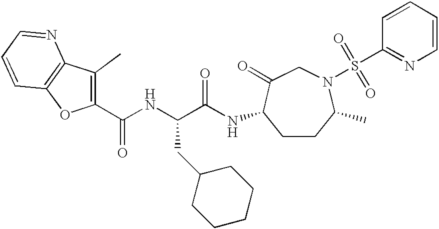
3-methyl-furo[3,2-b]-pyridine-2-carboxylic acid {(S)-2-cyclohexyl-1-[(4S,7R)-7-methyl-3-oxo-1-(pyridine-2-sulfonyl)-azepan-4-ylcarbamoyl]-ethyl}-amide; 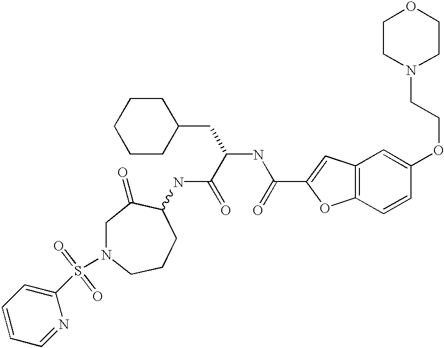
5-(2-morpholin-4-yl-ethoxy)-benzofuran-2-carboxylic acid {(S)-2-cyclohexyl-1-[3-oxo-1-(pyridine-2-sulfonyl)-azepan-4-ylcarbamoyl]-ethyl}-amide; 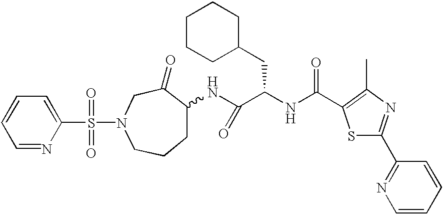
4-methyl-2-pyridin-2-yl-thiazole-5-carboxylic acid {(S)-2-cyclohexyl-1-[3-oxo-1-(pyridine-2-sulfonyl)-azepan-4-ylcarbamoyl]-ethyl}-amide; 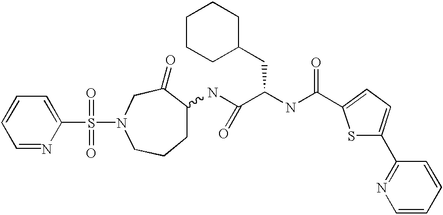
5-pyridin-2-yl-thiophene-2-carboxylic acid {(S)-2-cyclohexyl-1-[3-oxo-1-(pyridine-2-sulfonyl)-azepan-4-ylcarbamoyl]-ethyl}-amide; 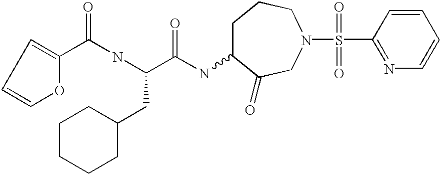
furan-2-carboxylic acid {(S)-2-cyclohexyl-1-[3-oxo-1-(pyridine-2-sulfonyl)-azepan-4-ylcarbamoyl]-ethyl}-amide; 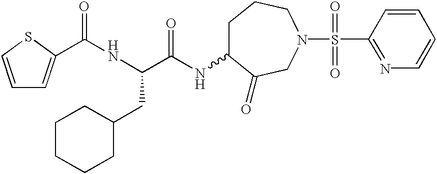
thiophene-2-carboxylic acid {(S)-2-cyclohexyl-1-[3-oxo-1-(pyridine-2-sulfonyl)-azepan-4-ylcarbamoyl]-ethyl}-amide; 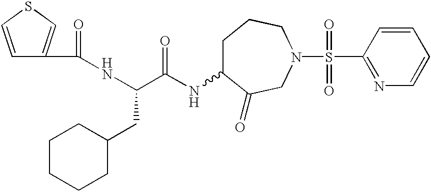
thiophene-3-carboxylic acid {(S)-2-cyclohexyl-1-[3-oxo-1-(pyridine-2-sulfonyl)-azepan-4-ylcarbamoyl]-ethyl}-amide; 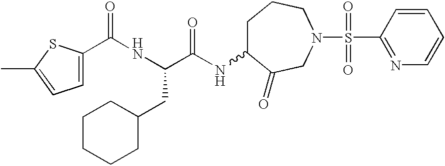
5-methyl-thiophene-2-carboxylic acid {(S)-2-cyclohexyl-1-[3-oxo-1-(pyridine-2-sulfonyl)-azepan-4-ylcarbamoyl]-ethyl}-amide; 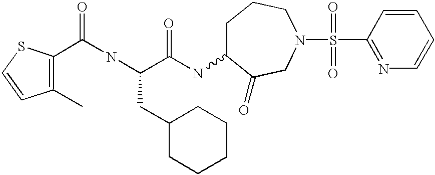
3-methyl-thiophene-2-carboxylic acid {(S)-2-cyclohexyl-1-[3-oxo-1-(pyridine-2-sulfonyl)-azepan-4-ylcarbamoyl]-ethyl}-amide; 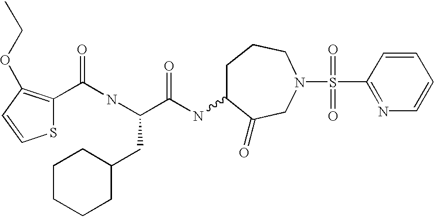
3-ethoxy-thiophene-2-carboxylic acid {(S)-2-cyclohexyl-1-[3-oxo-1-(pyridine-2-sulfonyl)-azepan-4-ylcarbamoyl]-ethyl}-amide; 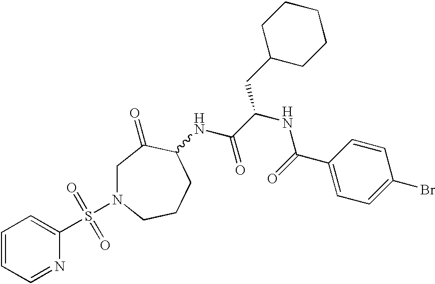
4-bromo-N-{(S)-2-cyclohexyl-1-[3-oxo-1-(pyridine-2-sulfonyl)-azepan-4-ylcarbamoyl]-ethyl}-benzamide; 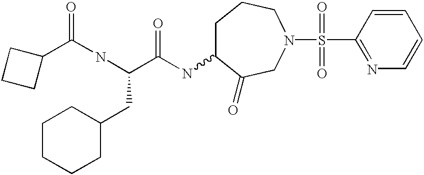
cyclobutanecarboxylic acid {(S)-2-cyclohexyl-1-[3-oxo-1-(pyridine-2-sulfonyl)-azepan-4-ylcarbamoyl]-ethyl}-amide; 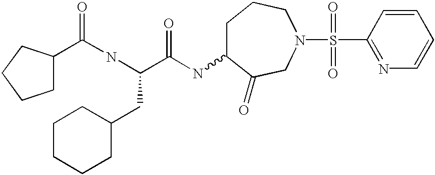
cyclopentanecarboxylic acid {(S)-2-cyclohexyl-1-[3-oxo-1-(pyridine-2-sulfonyl)-azepan-4-ylcarbamoyl]-ethyl}-amide; 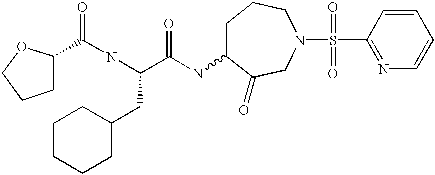
(S)-tetrahydro-furan-2-carboxylic acid {(S)-2-cyclohexyl-1-[3-oxo-1-(pyridine-2-sulfonyl)-azepan-4-ylcarbamoyl]-ethyl}-amide; 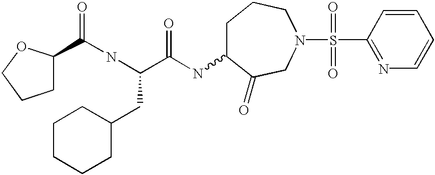
(R)-tetrahydro-furan-2-carboxylic acid {(S)-2-cyclohexyl-1-[3-oxo-1-(pyridine-2-sulfonyl)-azepan-4-ylcarbamoyl]-ethyl}-amide; 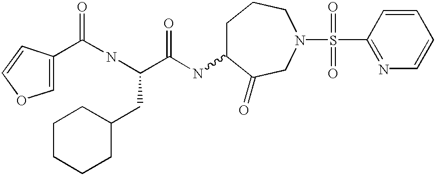
furan-3-carboxylic acid {(S)-2-cyclohexyl-1-[3-oxo-1-(pyridine-2-sulfonyl)-azepan-4-ylcarbamoyl]-ethyl}-amide; 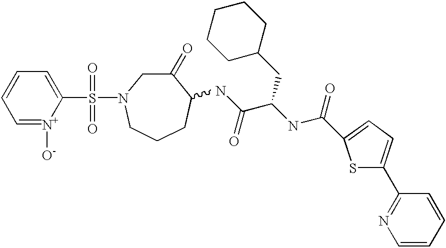
5-pyridin-2-yl-thiophene-2-carboxylic acid {(S)-2-cyclohexyl-1-[3-oxo-1-(1-oxy-pyridine-2-sulfonyl)-azepan-4-ylcarbamoyl]-ethyl}-amide; 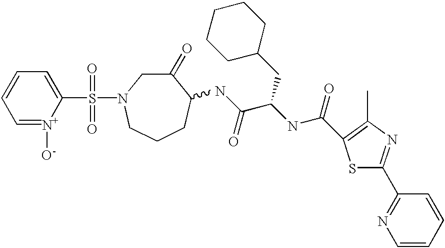
4-methyl-2-pyridin-2-yl-thiazole-5-carboxylic acid {(S)-2-cyclohexyl-1-[3-oxo-1-(1-oxy-pyridine-2-sulfonyl)-azepan-4-ylcarbamoyl]-ethyl}-amide; 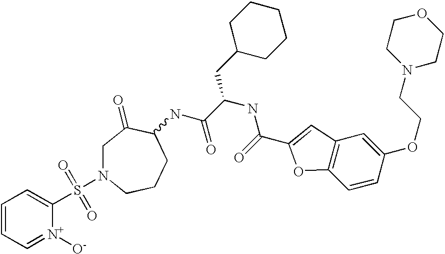
5-(2-morpholin-4-yl-ethoxy)-benzofuran-2-carboxylic acid {(S)-2-cyclohexyl-1-[3-oxo-1-(1-oxy-pyridine-2-sulfonyl)-azepan-4-ylcarbamoyl]-ethyl}-amide; 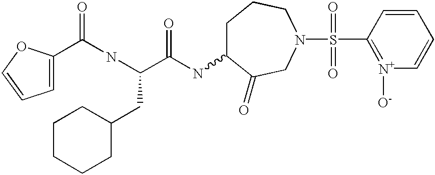
furan-2-carboxylic acid {(S)-2-cyclohexyl-1-[3-oxo-1-(1-oxy-pyridine-2-sulfonyl)-azepan-4-ylcarbamoyl]-ethyl}-amide; 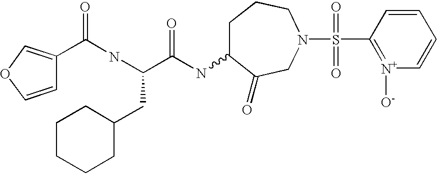
furan-3-carboxylic acid {(S)-2-cyclohexyl-1-[3-oxo-1-(1-oxy-pyridine-2-sulfonyl)-azepan-4-ylcarbamoyl]-ethyl}-amide; 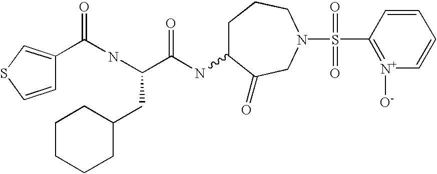
thiophene-3-carboxylic acid {(S)-2-cyclohexyl-1-[3-oxo-1-(1-oxy-pyridine-2-sulfonyl)-azepan-4ylcarbamoyl]-ethyl)-amide; 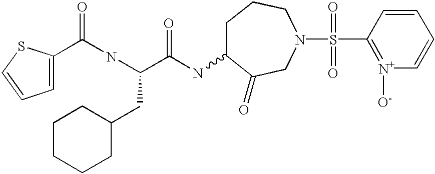
thiophene-2-carboxylic acid {(S)-2-cyclohexyl-1-[3-oxo-1-(1-oxy-pyridine-2-sulfonyl)-azepan-4-ylcarbamoyl]-ethyl}-amide; 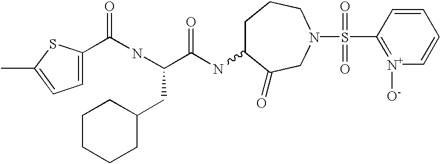
5-methyl-thiophene-2-carboxylic acid {(S)-2-cyclohexyl-1-[3-oxo-1-(1-oxy-pyridine-2-sulfonyl)-azepan-4-ylcarbamoyl]-ethyl}-amide; 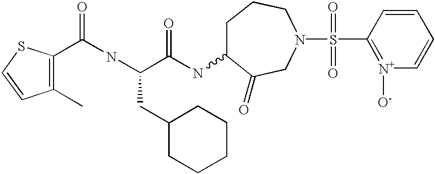
3-methyl-thiophene-2-carboxylic acid {(S)-2-cyclohexyl-1-[3-oxo-1-(1-oxy-pyridine-2-sulfonyl)-azepan-4-ylcarbamoyl]-ethyl}-amide; 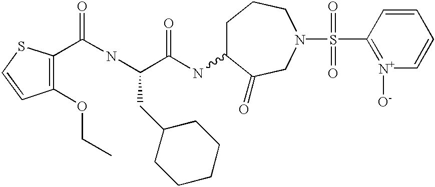
3-ethoxy-thiophene-2-carboxylic acid {(S)-2-cyclohexyl-1-[3-oxo-1-(1-oxy-pyridine-2-sulfonyl)-azepan-4-ylcarbamoyl]-ethyl}-amide; 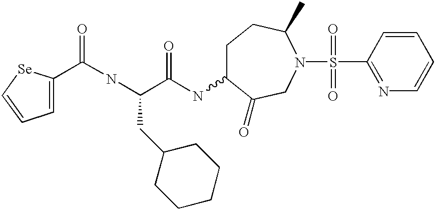
selenophene-2-carboxylic acid {(S)-2-cyclohexyl-1-[(R)-7-methyl-3-oxo-1-(pyridine-2-sulfonyl)-azepan-4-ylcarbamoyl]-ethyl}-amide; 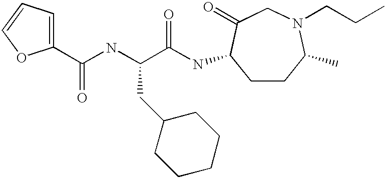
furan-2-carboxylic acid [(S)-2-cyclohexyl-1-((4S,7R)-7-methyl-3-oxo-1-propyl-azepan-4-ylcarbamoyl)-ethyl]-amide; 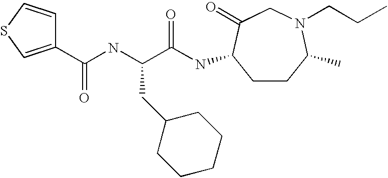
thiophene-3-carboxylic acid [(S)-2-cyclohexyl-1-((4S,7R)-7-methyl-3-oxo-1-propyl-azepan-4-ylcarbamoyl)-ethyl]-amide; 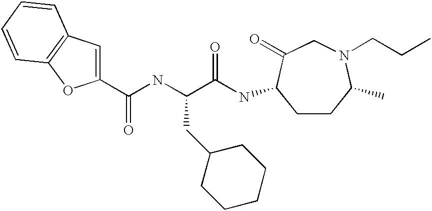
benzofuran-2-carboxylic acid [(S)-2-cyclohexyl-1-((4S,7R)-7-methyl-3-oxo-1-propyl-azepan-4-ylcarbamoyl)-ethyl]-amide; 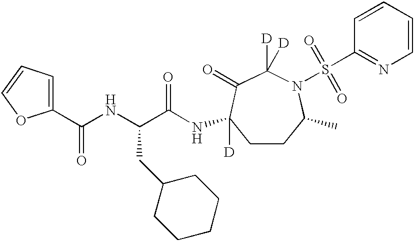
2,2,4-trideutero-Furan-2-carboxylic acid {(S)-2-cyclohexyl-1-[(4S,7R)-7-methyl-3-oxo-1-(pyridine-2-sulfonyl)-azepan-4-ylcarbamoyl]-ethyl}-amide; 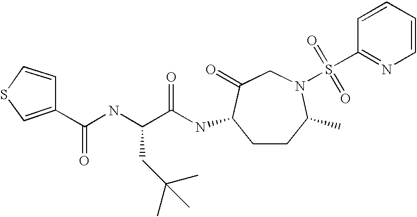
thiophene-3-carboxylic acid {(S)-3,3-dimethyl-1-[(4S,7R)-7-methyl-3-oxo-1-(pyridine-2-sulfonyl)-azepan-4-ylcarbamoyl]-butyl}-amide; 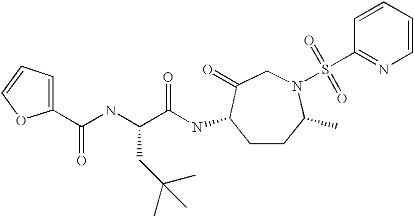
furan-2-carboxylic acid {(S)-3,3-dimethyl-1-[(4S,7R)-7-methyl-3-oxo-1-(pyridine-2sulfonyl)-azepan-4-ylcarbamoyl]-butyl}-amide; and 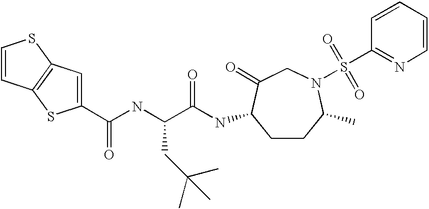
thieno[3,2-b]thiophene-2-carboxylic acid {(S)-3,3-dimethyl-1-[(4S,7R)-7-methyl-3-oxo-1-(pyridine-2-sulfonyl)-azepan-4-ylcarbamoyl]-butyl}-amide.

35. A compound according to claim 34 selected from the group consisting of: 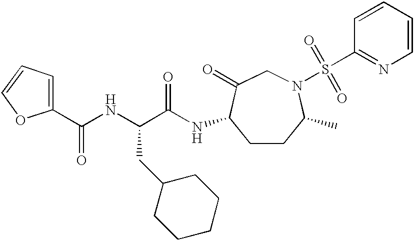
furan-2-carboxylic acid {(S)-2-cyclohexyl-1-[(4S,7R)-7-methyl-3-oxo-1-(pyridine-2-sulfonyl)-azepan-4-ylcarbamoyl]-ethyl}-amide; and 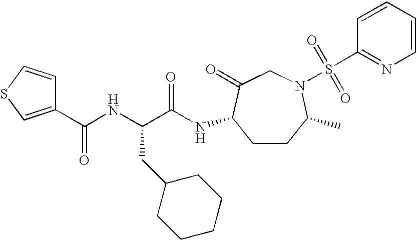
thiophene-3-carboxylic acid {(S)-2-cyclohexyl-1-[(4S,7R)-7-methyl-3-oxo-1-(pyridine-2-sulfonyl)-azepan-4-ylcarbamoyl]-ethyl}-amide.

36. A compound according to claim 35 which is: 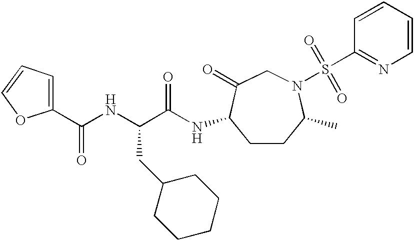
furan-2-carboxylic acid {(S)-2-cyclohexyl-1-[(4S,7R)-7-methyl-3-oxo-1-(pyridine-2-sulfonyl)-azepan-4-ylcarbamoyl]-ethyl}-amide.

37. A method of treatment and prevention of an autoimmune disease comprising inhibiting overexpression of cathepsin S by administering to a patient in need thereof an effective amount of a compound according to any one of claims 1 to 36.

38. A method according to claim 37 wherein said disease is selected from the group consisting of: rheumatoid arthritis, multiple sclerosis, juvenile-onset diabetes, sytemic lupus erythematosus, discoid lupus erythematosus, pemphigus vulgaris, pemphigoid, Grave's disease, myasthenia gravis, Hashimoto's thyroiditis, scleroderna, dermatomysositis, Addison's disease, pernicious anemia, primary myxoedema, thyrotoxicosis, autoimmune atrophic gastritis, stiff-man syndrome, Goodpasture's syndrome, sympathetic opthalamia, phacogenic uveitis, autoimmune haemolytic anaemia, idiopathic thrombocytopenic purpura, idiopathic leucopenia, primary biliary cirrhosis, active chronic hepatitis, cryptogenic cirrhosis, ulcerative colitis, Sjogren's syndrome, and mixed connective tissue diease.

39. A method of treatment or prevention of a disease state caused by the formation or complications of atherosclerotic lesions comprising inhibiting formation of said lesions or complications thereof by administering to a patient in need thereof an effective amount of a compound according to any one of claims 1 to 36.

40. A method of treatment of a disease which requires for therapy inhibition of a class II MHC-restricted immune response, comprising inhibiting said class II MHC-restricted immune response by administering to a patient in need thereof an effective amount of a compound according to any one of claims 1 to 36.

41. A method of treatment of a disease which requires for therapy inhibition of an asthmatic response, comprising inhibiting said asthmatic response by administering to a patient in need thereof an effective amount of a compound according to any one of claims 1 to 36.

42. A method of treatment of a disease which requires for therapy inhibition of an allergic response, comprising inhibiting said allergic response by administering to a patient in need thereof an effective amount of a compound according to any one of claims 1 to 36.

43. A method of treatment of a disease which requires for therapy inhibition of an immune response against a transplanted organ or tissue, comprising inhibiting said immune response against a transplanted organ or tissue by administering to a patient in need thereof an effective amount of a compound according to any one of claims 1 to 36.

44. A method of treatment of a disease which requires for therapy inhibition of elastase activity in atheroma, comprising inhibiting said elastase activity in atheroma by administering to a patient in need thereof an effective amount of a compound according to any one of claims 1 to 36.

45. Use of a compound according to any one of claims 1 to 36 in the manufacture of a medicament for use in inhibiting cathepsin S.

46. Use of a compound according to any one of claims 1 to 36 in the manufacture of a medicament for use in treatment and prevention of an autoimmune disease.

47. A use according to claim 46 wherein said disease is selected from the group consisting of: rheumatoid arthritis, multiple sclerosis, juvenile-onset diabetes, sytemic lupus erythematosus, discoid lupus erythematosus, pemphigus vulgaris, pemphigoid, Grave's disease, myasthenia gravis, Hashimoto's thyroiditis, scleroderma, dermatomysositis, Addison's disease, pernicious anemia, primary myxoedema, thyrotoxicosis, autoimmune atrophic gastritis, stiff-man syndrome, Goodpasture's syndrome, sympathetic opthalamia, phacogenic uveitis, autoimmune haemolytic anaemia, idiopathic thrombocytopenic purpura, idiopathic leucopenia, primary biliary cirrhosis, active chronic hepatitis, cryptogenic cirrhosis, ulcerative colitis, Sjogren's syndrome, and mixed connective tissue diease.

48. Use of a compound according to any one of claims 1 to 36 in the manufacture of a medicament for use in treatment or prevention of a disease state caused by the formation or complications of atherosclerotic lesions.

49. Use of a compound according to any one of claims 1 to 36 in the manufacture of a medicament for use in treatment of a disease which requires for therapy inhibition of a class II MHC-restricted immune response.

50. Use of a compound according to any one of claims 1 to 36 in the manufacture of a medicament for use in inhibition of an asthmatic response.

51. Use of a compound according to any one of claims 1 to 36 in the manufacture of a medicament for use in inhibition of an allergic response.

52. Use of a compound according to any one of claims 1 to 36 in the manufacture of a medicament for use in inhibition of an immune response against a transplanted organ or tissue.

53. Use of a compound according to any one of claims 1 to 36 in the manufacture of a medicament for use in inhibition of elastase activity in atheroma.

54. A compound selected from the group consisting of: 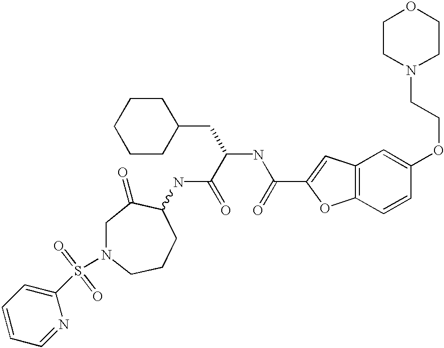
5-(2-morpholin-4-yl-ethoxy)-benzofuran-2-carboxylic acid {(S)-2-cyclohexyl-1-[3-oxo-1-(pyridine-2-sulfonyl)-azepan-4-ylcarbamoyl]-ethyl}-amide; 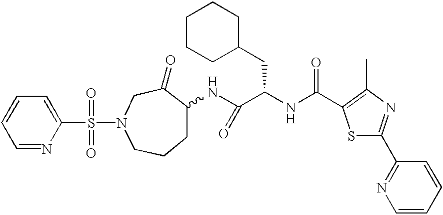
4-methyl-2-pyridin-2-yl-thiazole-5-carboxylic acid {(S)-2-cyclohexyl-1-[3-oxo-1-(pyridine-2-sulfonyl)-azepan-4-ylcarbamoyl]-ethyl}-amide; 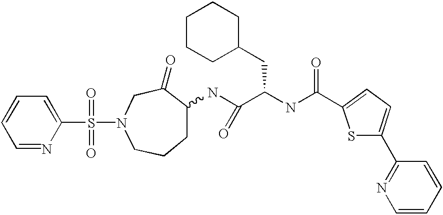
5-pyridin-2-yl-thiophene-2-carboxylic acid {(S)-2-cyclohexyl-1-[3-oxo-1-(pyridine-2-sulfonyl)-azepan-4-ylcarbamoyl]-ethyl}-amide; 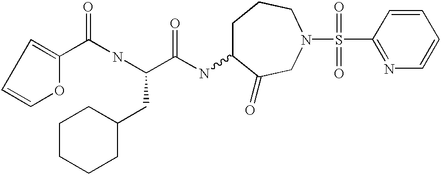
furan-2-carboxylic acid {(S)-2-cyclohexyl-1-[3-oxo-1-(pyridine-2-sulfonyl)-azepan-4-ylcarbamoyl]-ethyl}-amide; 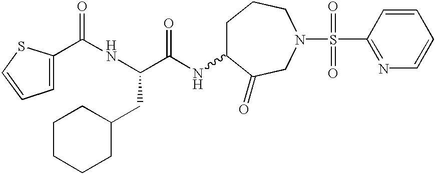
thiophene-2-carboxylic acid {(S)-2-cyclohexyl-1-[3-oxo-1-(pyridine-2-sulfonyl)-azepan-4-ylcarbamoyl]-ethyl}-amide; 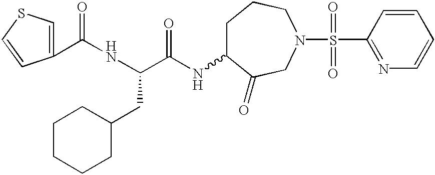
thiophene-3-carboxylic acid {(S)-2-cyclohexyl-1-[3-oxo-1-(pyridine-2-sulfonyl)-azepan-4-ylcarbamoyl]-ethyl}-amide; 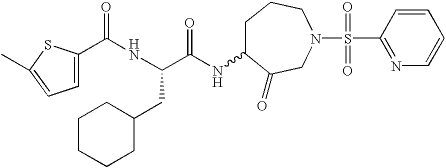
5-methyl-thiophene-2-carboxylic acid {(S)-2-cyclohexyl-1-[3-oxo-1-(pyridine-2-sulfonyl)-azepan-4-ylcarbamoyl]-ethyl}-amide; 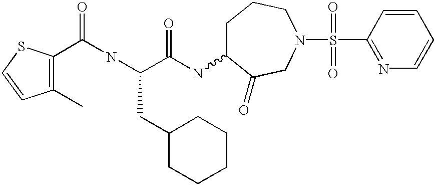
3-methyl-thiophene-2-carboxylic acid {(S)-2-cyclohexyl-1-[3-oxo-1-(pyridine-2-sulfonyl)-azepan-4-ylcarbamoyl]-ethyl}-amide; 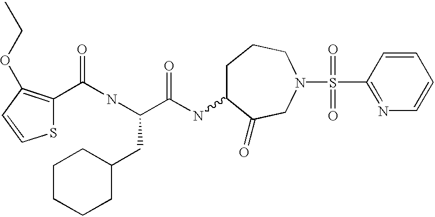
3-ethoxy-thiophene-2-carboxylic acid {(S)-2-cyclohexyl-1-[3-oxo-1-(pyridine-2-sulfonyl)-azepan-4-ylcarbamoyl]-ethyl}-amide; 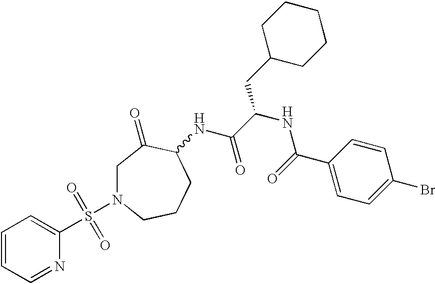
4-bromo-N-{(S)-2-cyclohexyl-1-[3-oxo-1-(pyridine-2-sulfonyl)-azepan-4-ylcarbamoyl]-ethyl}-benzamide; 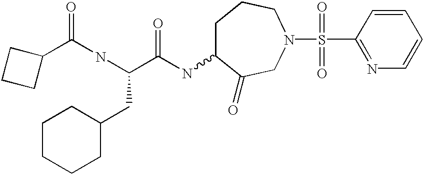
cyclobutanecarboxylic acid {(S)-2-cyclohexyl-1-[3-oxo-1-(pyridine-2-sulfonyl)-azepan-4-ylcarbamoyl]-ethyl}-amide; 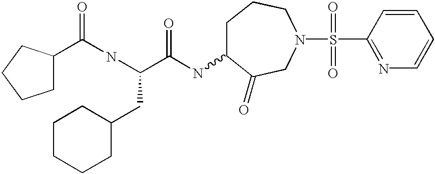
cyclopentanecarboxylic acid {(S)-2-cyclohexyl-1-[3-oxo-1-(pyridine-2-sulfonyl)-azepan-4-ylcarbamoyl]-ethyl}-amide; 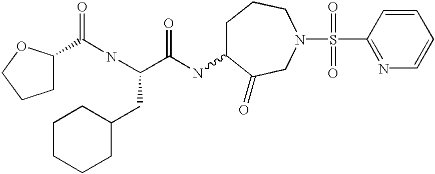
(S)-tetrahydro-furan-2-carboxylic acid {(S)-2-cyclohexyl-1-[3-oxo-1-(pyridine-2-sulfonyl)-azepan-4-ylcarbamoyl]-ethyl}-amide; 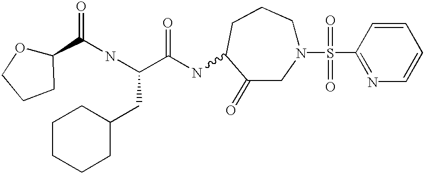
(R)-tetrahydro-furan-2-carboxylic acid {(S)-2-cyclohexyl-1-[3-oxo-1-(pyridine-2-sulfonyl)-azepan-4-ylcarbamoyl]-ethyl}-amide; 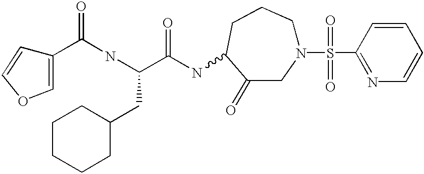
furan-3-carboxylic acid {(S)-2-cyclohexyl-1-[3-oxo-1-(pyridine-2-sulfonyl)-azepan-4-ylcarbamoyl]-ethyl}-amide; 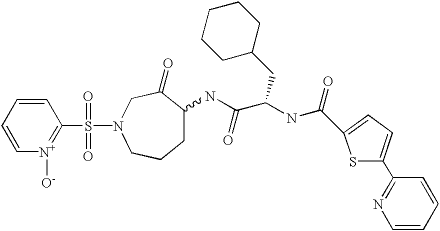
5-pyridin-2-yl-thiophene-2-carboxylic acid {(S)-2-cyclohexyl-1-[3-oxo-1-(1-oxy-pyridine-2-sulfonyl)-azepan-4-ylcarbamoyl]-ethyl}-amide; 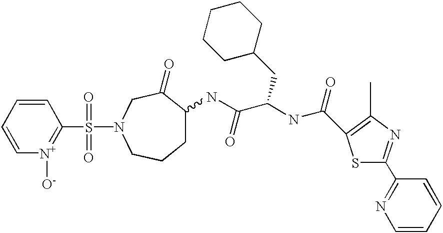
4-methyl-2-pyridin-2-yl-thiazole-5-carboxylic acid {(S)-2-cyclohexyl-1-[3-oxo-1-(1oxy-pyridine-2-sulfonyl)-azepan-4-ylcarbamoyl]-ethyl}-amide; 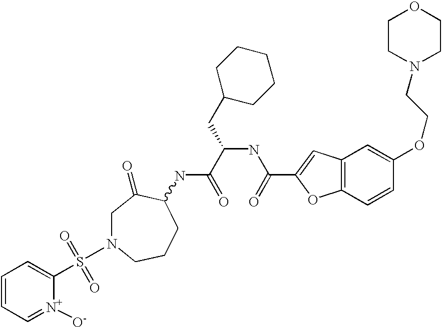
5-(2-morpholin-4-yl-ethoxy)-benzofuran-2-carboxylic acid {(S)-2-cyclohexyl-1-[3-oxo-1-(1-oxy-pyridine-2-sulfonyl)-azepan-4-ylcarbamoyl]-ethyl}-amide; 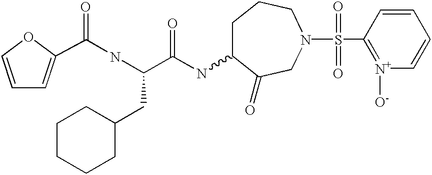
furan-2-carboxylic acid {(S)-2-cyclohexyl-1-[3-oxo-1-(1-oxy-pyridine-2-sulfonyl)-azepan-4-ylcarbamoyl]-ethyl}-amide; 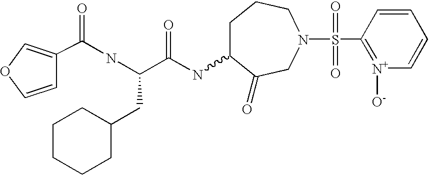
furan-3-carboxylic acid {(S)-2-cyclohexyl-1-[3-oxo-1-(1-oxy-pyridine-2-sulfonyl)-azepan-4-ylcarbamoyl]-ethyl}-amide; 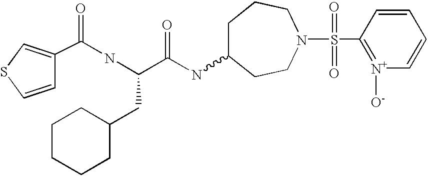
thiophene-3-carboxylic acid {(S)-2-cyclohexyl-1-[3-oxo-1-(1-oxy-pyridine-2-sulfonyl)-azepan-4-ylcarbamoyl]-ethyl}-amide; 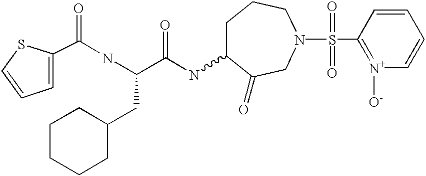
thiophene-2-carboxylic acid {(S)-2-cyclohexyl-1-[3-oxo-1-(1-oxy-pyridine-2-sulfonyl)-azepan-4-ylcarbamoyl]-ethyl}-amide; 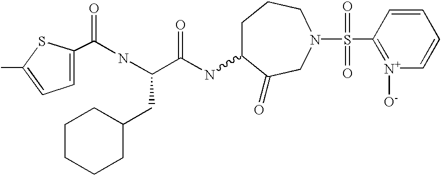
5-methyl-thiophene-2-carboxylic acid {(S)-2-cyclohexyl-1-[3-oxo-1-(1-oxy-pyridine-2-sulfonyl)-azepan-4-ylcarbamoyl]-ethyl}-amide; 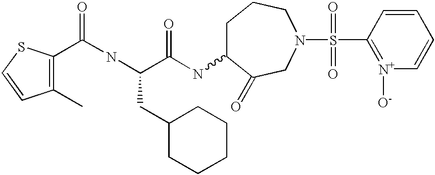
3-methyl-thiophene-2-carboxylic acid {(S)-2-cyclohexyl-1-[3-oxo-1-(1-oxy-pyridine-2-sulfonyl)-azepan-4-ylcarbamoyl]-ethyl}-amide; 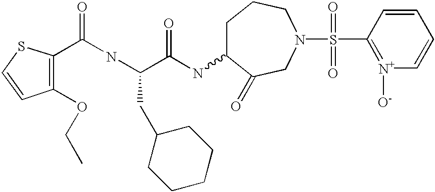
3-ethoxy-thiophene-2-carboxylic acid {(S)-2-cyclohexyl-1-[3-oxo-1-(1-oxy-pyridine-2-sulfonyl)-azepan-4-ylcarbamoyl]-ethyl}-amide; 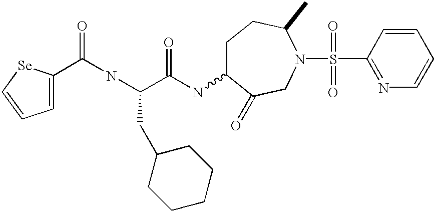
selenophene-2-carboxylic acid {(S)-2-cyclohexyl-1-[(R) -7-methyl-3-oxo-1-(pyridine-2-sulfonyl)- azepan-4-ylcarbamoyl]-ethyl}-amide; and 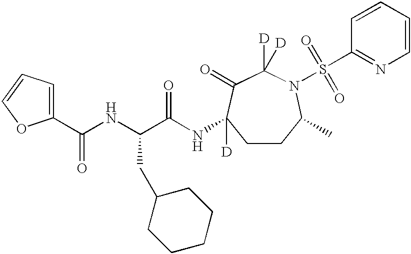
2,2,4-trideutero-Furan-2-carboxylic acid {(S)-2-cyclohexyl-1-[(4S,7R)-7-methyl-3-oxo-1-(pyridine-2-sulfonyl)-azepan-4-ylcarbamoyl]-ethyl}-amide.
